SEQUENCE LISTING (73 sequences)

SEQ ID NO: 1 - protein, 1255 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 2 - DNA, 3768 bp, SARS coronavirus
atgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttggcaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacatttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgcgcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagctcgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataa

SEQ ID NO: 3 - DNA, 29 bp, Artificial Sequence
  A synthetic primer
agtcggatccggtaggcttatcattagag

SEQ ID NO: 4 - DNA, 20 bp, Artificial Sequence
  A synthetic primer
ccatcaggggagaaaggcac

SEQ ID NO: 5 - DNA, 20 bp, Artificial Sequence
  A synthetic primer
gtgcctttctcccctgatgg

SEQ ID NO: 6 - DNA, 19 bp, Artificial Sequence
  A synthetic primer
gaagagcagcgccagcacc

SEQ ID NO: 7 - DNA, 19 bp, Artificial Sequence
  A synthetic primer
ggtgctggcgctgctcttc

SEQ ID NO: 8 - DNA, 28 bp, Artificial Sequence
  A synthetic primer
actgtctagagttcgtttatgtgtaatg

SEQ ID NO: 9 - DNA, 29 bp, Artificial Sequence
  A synthetic primer
agtcggatccgaccggtgcaccacttttg

SEQ ID NO: 10 - DNA, 28 bp, Artificial Sequence
  A synthetic primer
agtcgggcccctgttcagcagcaatacc

SEQ ID NO: 11 - DNA, 28 bp, Artificial Sequence
  A synthetic primer
actgggatccgaagtgttcgctcaagtc

SEQ ID NO: 12 - DNA, 26 bp, Artificial Sequence
  A synthetic primer
actgtctagattgctcatattttccc

SEQ ID NO: 13 - protein, 740 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQ

SEQ ID NO: 14 - protein, 429 aa, SARS coronavirus
EVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 15 - protein, 1170 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDEVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 16 - protein, 21 aa, Artificial Sequence
  A synthetic k chain leader sequence
METDTLLLWVLLLWVPGSTGD

SEQ ID NO: 17 - protein, 10 aa, Artificial Sequence
  A synthetic myc epitope
EQKLISEEDL

SEQ ID NO: 18 - protein, 6 aa, Artificial Sequence
  A synthetic histidine tag
HHHHHH

SEQ ID NO: 19 - DNA, 24 bp, Artificial Sequence
  A synthetic primer
ctagctcgagcaacagcatctgtg

SEQ ID NO: 20 - protein, 100 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRG

SEQ ID NO: 21 - protein, 100 aa, SARS coronavirus
WVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGY

SEQ ID NO: 22 - protein, 100 aa, SARS coronavirus
QPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIY

SEQ ID NO: 23 - protein, 100 aa, SARS coronavirus
QTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPG

SEQ ID NO: 24 - protein, 100 aa, SARS coronavirus
QTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLS

SEQ ID NO: 25 - protein, 100 aa, SARS coronavirus
FELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQD

SEQ ID NO: 26 - protein, 100 aa, SARS coronavirus
VNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNF

SEQ ID NO: 27 - protein, 100 aa, SARS coronavirus
SISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFI

SEQ ID NO: 28 - protein, 100 aa, SARS coronavirus
EDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYE

SEQ ID NO: 29 - protein, 100 aa, SARS coronavirus
NQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEI

SEQ ID NO: 30 - protein, 100 aa, SARS coronavirus
RASANLAATKMSECVLGQSKRVDFCGKGYHLMSFPQAAPHGVVFLHVTYVPSQERNFTTAPAICHEGKAYFPREGVFVFNGTSWFITQRNFFSPQIITTD

SEQ ID NO: 31 - protein, 90 aa, SARS coronavirus
NTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 32 - protein, 200 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGY

SEQ ID NO: 33 - protein, 200 aa, SARS coronavirus
QPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPG

SEQ ID NO: 34 - protein, 200 aa, SARS coronavirus
QTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQD

SEQ ID NO: 35 - protein, 200 aa, SARS coronavirus
VNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFI

SEQ ID NO: 36 - protein, 200 aa, SARS coronavirus
EDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEI

SEQ ID NO: 37 - protein, 190 aa, SARS coronavirus
RASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 38 - protein, 400 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPG

SEQ ID NO: 39 - protein, 600 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQD

SEQ ID NO: 40 - protein, 800 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFI

SEQ ID NO: 41 - protein, 1000 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEI

SEQ ID NO: 42 - protein, 1190 aa, SARS coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 43 - protein, 84 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRG

SEQ ID NO: 44 - protein, 184 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGY

SEQ ID NO: 45 - protein, 384 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPG

SEQ ID NO: 46 - protein, 584 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQD

SEQ ID NO: 47 - protein, 784 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFI

SEQ ID NO: 48 - protein, 984 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEI

SEQ ID NO: 49 - protein, 1174 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQ

SEQ ID NO: 50 - protein, 260 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAV

SEQ ID NO: 51 - protein, 430 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHG

SEQ ID NO: 52 - protein, 521 aa, SARS coronavirus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGV

SEQ ID NO: 53 - protein, 777 aa, Artificial Sequence
  Synthetic sequence of amino acids 17-757 of SEQ ID NO1 plus an N-terminal mouse K chain leader sequence and a C-histidine tag.
METDTLLLWVLLLWVPGSTGDDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQEQKLISEEDLHHHHHH

SEQ ID NO: 54 - protein, 297 aa, Artificial Sequence
  Synthetic sequence of amino acids 17-276 of SEQ ID NO1 plus an N-terminal mouse K chain leader sequence and a C-histidine tag.
METDTLLLWVLLLWVPGSTGDDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVEQKLISEEDLHHHHHH

SEQ ID NO: 55 - protein, 558 aa, Artificial Sequence
  A synthetic sequence of amino acids 17-537 of SEQ ID NO1 plus an N-terminal mouse K chain leader sequence and a C-terminal myc epitope and a poly histidine tag.
METDTLLLWVLLLWVPGSTGDDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVEQKLISEEDLHHHHHH

SEQ ID NO: 56 - protein, 739 aa, Artificial Sequence
  A synthetic sequence of amino acids 17-756 of SEQ ID NO1 without a signal peptide at the N-terminus
DRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCAFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAE

SEQ ID NO: 57 - protein, 265 aa, Artificial Sequence
  A synthetic sequence of amino acids 272-537 of SEQ ID NO1
ITDAVDCSQNPLAELKCSVKSFEIDKGIYQTSNFRVVPSGDVVRFNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGV

SEQ ID NO: 58 - protein, 17 aa, SARS coronavirus
DVQAPNYTQHTSSMRGC

SEQ ID NO: 59 - protein, 15 aa, SARS coronavirus
PSSKRFQPQQFGRDC

SEQ ID NO: 60 - protein, 16 aa, SARS coronavirus
MFIFLLFLTLTSGSDL

SEQ ID NO: 61 - protein, 235 aa, Artificial Sequence
  A synthetic sequence of amino acids 303-537 of SEQ ID NO1 containing the receptor binding domain
SNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGV

SEQ ID NO: 62 - protein, 199 aa, Artificial Sequence
  A synthetic sequence of amino acids 319-517 of SEQ ID NO1 containing the receptor binding domain
ITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 63 - protein, 200 aa, Artificial Sequence
  A synthetic sequence of amino acids 319-518 of SEQ ID NO1 containing the receptor binding domain
ITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTD

SEQ ID NO: 64 - DNA, 23 bp, Artificial Sequence
  A synthetic primer
gatcggatccggtacaatcacag

SEQ ID NO: 65 - DNA, 23 bp, Artificial Sequence
  A synthetic primer
gatcgggcccgacacactggttc

SEQ ID NO: 66 - protein, 197 aa, Artificial Sequence
  A synthetic sequence of amino acids 317-517 of SEQ ID NO1 containing the receptor binding domain.
NLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLST

SEQ ID NO: 67 - DNA, 29 bp, Artificial Sequence
  A synthetic primer.
actgtctagatggtaccgagctcggatcc

SEQ ID NO: 68 - DNA, 25 bp, Artificial Sequence
  A synthetic primer.
cagtagatctcgaggctgatcagcg

SEQ ID NO: 69 - protein, 11 aa, SARS coronavirus
GFYTTTGIGYQ

SEQ ID NO: 70 - protein, 9 aa, Homo sapiens
DTVMGGMDV

SEQ ID NO: 71 - protein, 10 aa, Homo sapiens
QVWDSSSDYV

SEQ ID NO: 72 - protein, 8 aa, SARS coronavirus
NDLCFSNV

SEQ ID NO: 73 - protein, 12 aa, SARS coronavirus
FELLNAPATVCG

(19) United States
(12) Patent Application Publication
(10) Pub. No.: US 2006/0240515 A1
(43) Pub. Date: Oct. 26, 2006

(54) Soluble fragments of the SARS-CoV spike glycoprotein

(76) Inventors: Dimiter S. Dimitrov, Frederick, MD (US); Xiaodong Xiao, Frederick, MD (US); Zhu Zhongyu, Frederick, MD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/335,197

(22) Filed: Jan. 19, 2006

Related U.S. Application Data
(63) Continuation-in-part of application No. PCT/US04/23345, filed on Jul. 20, 2004.
(60) Provisional application No. 60/489,166, filed on Jul. 21, 2003.
(60) Provisional application No. 60/524,642, filed on Nov. 25, 2003.

(51) Int. Cl. C12Q 1/68; C12P 21/06

(52) U.S. Cl. 435/69.1; 435/6

(57) ABSTRACT

The invention relates to the spike protein from the virus (SARS-CoV) that is etiologically linked to severe acute respiratory syndrome (SARS); polypeptides and peptide fragments of the spike protein; nucleic acid segments and constructs that encode the spike protein, polypeptides and peptide fragments of the spike protein, and coupled proteins that include the spike protein or a portion thereof; peptidomimetics; vaccines; methods for vaccination and treatment of severe acute respiratory syndrome; antibodies; aptamers; and kits containing immunological compositions, or antibodies (or aptamers) that bind to the spike protein.

This application is a national stage application of PCT application Ser. No. PCT/US2004/023345, which claims priority from U.S. Application Ser. No. 60/489,166 filed Jul. 21, 2003 and from U.S. Application Ser. No. 60/524,642 filed Nov. 25, 2003, the contents of which are hereby incorporated by reference in their entireties.

### GOVERNMENT FUNDING

The invention described herein was developed with the support of the Department of Health and Human Services. The United States Government has certain rights in the invention.

### FIELD OF THE INVENTION

The invention relates generally to a spike polypeptide that is encoded by a coronavirus (herein SARS-CoV), which is etiologically linked to Severe Acute Respiratory Syndrome (SARS). The invention further relates to nucleic acids and polypeptides having amino acid sequences that correspond to fragments of spike protein of SARS-CoV, and conservative variants thereof. The invention also relates to use of these nucleic acids, polypeptides, variants, and fragments to produce antibodies that recognize the spike protein of SARS-CoV, and for the production of vaccines against SARS. Another aspect of the invention relates to spike protein fragments for inhibiting fusion of the SARS-CoV with animal cells.

### BACKGROUND OF THE INVENTION

Severe acute respiratory syndrome (SARS) is an infectious atypical pneumonia that has recently been recognized in patients in 32 countries and regions. The atypical pneumonia with unknown etiology was initially observed in Guangdong Province, China. This observation was followed by reports from Hong Kong, Vietnam, Singapore, Canada and Beijing of severe febrile respiratory illness that spread to household members and health care workers. This disease was later designated “severe acute respiratory syndrome (SARS)” by the World Health Organization (WHO). Until May 19, 2003, a cumulative total of 7,864 SARS cases were reported to WHO from 29 countries. A total of 643 deaths (case-fatality proportion: 8.2%) were reported.

Researchers around the world have sequenced the genome of SARS causing viruses from different regions of the globe. The viruses have been classified as coronaviruses. Coronaviruses have been grouped into three categories based on cross-reactivity of antibodies backed up by genetic data. Two previously known human viruses fell into different groups than SARS-CoV. The coronavirus that causes SARS does not fit into any of the previously known clusters. Rather, it forms a new group by itself. Phylogenetic analysis of the predicted viral proteins indicates that the virus does not closely resemble any of the three previously known groups of coronaviruses. Most coronaviruses cause either a respiratory or an enteric disease, which is also transmitted by the faecal-oral route.

The incubation period for SARS is usually 2 to 7 days. Infection is characterized by fever, non-productive cough, shortness of breath, and the presence of minimal auscultatory findings with consolidation on chest radiographs. Lymphopenia, leucopenia, thrombocytopenia, and elevated liver enzymes and creatinine kinase may also be present in most cases. Symptoms relating to the gastrointestinal tract were also noticed in SARS patients.

Pathological studies of patients who died of SARS from Guangdong, Hongkong, Beijing and Singapore showed diffuse alveolar damage (DAD) in the lung as the most notable feature. In those individuals with severe disease resulting in death, scattered type II pneumocytes showed marked cytologic changes that include multinucleation, cytomegaly, nucleomegaly, clearing of nuclear chromatin, and prominent nucleoli. Although these changes were severe, they were within the spectrum of epithelial changes seen in other cases of diffuse alveolar damage. Morphologic changes that were identified included bronchial epithelial denudation, loss of cilia, and squamous metaplasia. Other findings included focal intraalveolar hemorrhage, hemophagocytosis, necrotic inflammatory debris in small airways, organizing pneumonia or secondary bacterial pneumonia.

The pathogenesis of this disorder remains to be determined. However, the mechanism of acute lung injury could involve direct damage by the virus to the alveolar wall by targeting either endothelial cells or epithelial cells. Alternatively, the virus could infect inflammatory cells with the injury mediated through cytokines, interleukins, or tumor necrosis factor-alpha. It is also possible that the tissue damage in SARS is not directly related to viral infection in tissues but is a secondary effect of cytokines or other factors induced by viral infection proximal to but not within the lung tissue.

Pathologic evaluation of the fatal cases showed that hepatocytes underwent fatty degeneration, cloudy swelling, apoptosis and dot necrosis, with Kupffer cell proliferation and portal infiltrates of lymphocytes. There were regional hemorrhages, vascular congestion and lymphocytic infiltration in gastrointestinal walls of the patient.

Due to the ability of SARS-CoV to be spread through an airborne route, SARS-CoV presents a particular threat to the health of large populations of people throughout the world. Accordingly, methods to immunize people before infection, diagnose infection, immunize people during infection, and treat infected persons infected with SARS-CoV are greatly needed.

### SUMMARY OF THE INVENTION

These and other needs are met by the invention described herein. The invention provides polypeptides; peptide fragments; viral fusion inhibitors; coupled proteins; immunopeptides; immune compositions; peptidomimetics; nucleic acid segments; expression cassettes; nucleic acid constructs; recombinant viruses; viral vaccines; peptide vaccines; microorganism vaccines; DNA vaccines; antibodies; aptamers; pharmaceutical compositions; methods to immunize an animal; a method to treat severe acute respiratory syndrome (SARS); methods to diagnose SARS; and kits.

The invention provides polypeptides having an amino acid sequence corresponding to that of a polypeptide that is etiologically linked to SARS. Preferably the polypeptide is the spike protein from SARS-CoV that can inhibit SARS fusion with animal cells and/or raise an immune response against SARS-CoV in an animal. In some embodiments, the polypeptide is a soluble form of the spike protein from SARS-CoV. In other embodiments, the polypeptide includes amino acids 17-757 of the spike protein from SARS-CoV. In some embodiments, the polypeptide includes amino acids 762-1189 of the spike protein from SARS-CoV. In other embodiments, the polypeptide includes amino acids 17-757 of the spike protein from SARS-CoV. In some embodiments, the polypeptide includes amino acids 17-276 of the spike protein from SARS-CoV. In other embodiments, the polypeptide includes amino acids 303-537 of the spike protein from SARS-CoV. In some embodiments, the polypeptide includes amino acids 317-517 of the spike protein from SARS-CoV. In other embodiments, the polypeptide includes amino acids 272-537 of the spike protein from SARS-CoV. In some embodiments, the polypeptide includes amino acids 17-537 of the spike protein from SARS-CoV. In other embodiments, the polypeptide includes amino acids 17-1189 (relative to SEQ ID NO: 1) of the spike protein from SARS-CoV. The polypeptides of the invention can inhibit SARS-CoV fusion with animal cells. The nucleic acids and polypeptides of the invention can elicit an immune response when used to inoculate an animal. In some embodiments, the nucleic acids and polypeptides of the invention elicit a cellular immune response when used to inoculate an animal. In other embodiments, the nucleic acids and polypeptides of the invention elicit a humoral immune response when used to inoculate an animal. The animal can be a reptile. In some embodiments, the animal is an avian. In other embodiments, the animal is a mammal. Sometimes, the animal is a human.

The invention provides peptide fragments of the spike protein from SARS-CoV. Preferably the peptide fragments are soluble in aqueous solution. A peptide fragment of the invention may lack one amino acid residue from the amino acid sequence of the full length spike protein from SARS-CoV. In some embodiments, peptide fragments are at least three amino acids in length. In other embodiments, peptide fragments are at least 10 amino acids in length. In some embodiments, peptide fragments are at least 20 amino acids in length. In other embodiments, peptide fragments are at least 30 amino acids in length. In some embodiments, peptide fragments are at least 40 amino acids in length. In other embodiments, peptide fragments are at least 50 amino acids in length. In some embodiments, peptide fragments are at least 60 amino acids in length. The peptide fragments may also be single amino acid unit additions to a fragment of a given length. For example, peptide fragment may be 3, 4, 10, 11, 21, 22, 31, or 32 amino acids in length. The peptide fragments of the invention can inhibit SARS Co-V fusion with animal cells or elicit an immune response when used to inoculate an animal. Examples of peptides that can elicit an immune response after inoculation of an animal include, for example, the D24 peptide having sequence DVQAPNYTQHTSSMRGC (SEQ ID NO:58), the P540 peptide having sequence PSSKRFQPQQFGRDC (SEQ ID NO:59) and the peptide GFYTTTGIGYQ (SEQ ID NO:69). In some embodiments, the peptide fragments of the invention elicit a cellular immune response when used to inoculate an animal. In other embodiments, the peptide fragments of the invention elicit a humoral immune response when used to inoculate an animal. The animal can be a reptile. In some embodiments, the animal is an avian. In other embodiments, the animal is a mammal. In further embodiments, the animal is a human.

The invention provides coupled proteins. The coupled proteins include a carrier protein that is coupled to a second polypeptide. Preferably, the carrier protein is soluble. In some embodiments, the carrier protein increases an immune response to the second polypeptide of the coupled protein when used to inoculate an animal. In other embodiments, the carrier protein elicits a cellular immune response to the second polypeptide of the coupled protein when used to inoculate an animal. In some embodiments, the carrier protein elicits a humoral immune response to the second polypeptide of the coupled protein when used to inoculate an animal. The second polypeptide can be a polypeptide or a peptide fragment of the invention, or a conservative variant thereof. The animal can be a reptile. In some embodiments, the animal is an avian. In other embodiments, the animal is a mammal. In further embodiments, the animal is a human.

The invention provides immunopeptides that include a polypeptide or peptide fragment of the invention, or a conservative variant thereof, that is coupled to an acetyl group, a picryl group, an arsanilic acid, or to a sulfanilic acid. In some embodiments, the immunopeptide is coupled to an acetyl or a picryl group. In other embodiments, immunopeptide is coupled to arsanilic acid or sulfanilic acid. Preferably, the immunopeptide is soluble. Preferably, the immunopeptide elicits an immune response when used to inoculate an animal. In some embodiments, the immunopeptide elicits a humoral immune response when used to inoculate an animal. In other embodiments, the immunopeptide elicits a cellular immune response when used to inoculate an animal. The animal can be a reptile. In some embodiments, the animal is an avian. In other embodiments, the animal is a mammal. In further embodiments, the animal is a human.

The invention provides peptidomimetics that are polypeptides or peptide fragments of the invention, and conservative variants thereof, in which a peptide bond has been replaced with a non-peptide bond. In some embodiments, the peptidomimetic can inhibit SARS Co-V fusion with animal cells. In other embodiments, the peptidomimetic elicits an immune response when used to inoculate an animal. For example, the peptidomimetic can elicit a cellular immune response when used to inoculate an animal. Alternatively, the peptidomimetic elicits a humoral immune response when used to inoculate an animal. The animal can be a reptile. In some embodiments, the animal is an avian. In other embodiments, the animal is a mammal. In further embodiments, the animal is a human.

The invention provides compositions containing an adjuvant and a nucleic acid, polypeptide, a peptide fragment, or a peptidomimetic of the invention. In some embodiments, the composition inhibits SARS-CoV fusion with animal cells. In other embodiments, the composition elicits an immune response when used to inoculate an animal. In some embodiments, the immune composition elicits a cellular immune response when used to inoculate an animal. In other embodiments, the immune composition elicits a humoral immune response when used to inoculate an animal. The animal can be a reptile. In some embodiments, the animal is an avian. In other embodiments, the animal is a mammal. In further embodiments, the animal is a human.

The invention provides nucleic acid segments that encode polypeptides and peptide fragments of the invention, and conservative variants thereof.

The invention provides expression cassettes having a promoter that is operably linked to a nucleic acid segment of the invention. In some embodiments, the promoter is constitutive. In other embodiments, the promoter is inducible.

The invention provides nucleic acid constructs that include a vector and a nucleic acid segment of the invention. The nucleic acid construct can include an expression cassette of the invention. In some embodiments, the vector can be a virus. In other embodiments, the vector is a plasmid. In further embodiments, the vector is an expression vector.

The invention provides a recombinant virus that includes a viral vector and a nucleic acid segment of the invention. In some embodiments, the viral vector is a herpes virus. In other embodiments, the viral vector is a canarypox virus. In other embodiments, the viral vector is an adenovirus. In further embodiments, the viral vector is a vaccinia virus.

The invention provides a viral vaccine against SARS that includes a viral vector, a nucleic acid segment of the invention, and a pharmaceutical carrier. In some embodiments, the viral vector is a herpes virus. In other embodiments, the viral vector is a canarypox virus. In other embodiments, the viral vector is an adenovirus. In further embodiments, the viral vector is a vaccinia virus. Preferably, the pharmaceutical carrier is formulated for injection. Preferably, the viral vaccine elicits an immune response when used to inoculate an animal. In some embodiments, the viral vaccine elicits a cellular immune response when used to inoculate an animal. In other embodiments, the viral vaccine elicits a humoral immune response when used to inoculate an animal. The animal can be a reptile. In some embodiments, the animal is an avian. In other embodiments, the animal is a mammal. In further embodiments, the animal is a human.

The invention provides a peptide vaccine against SARS that includes a peptidomimetic, polypeptide or a peptide fragment of the invention, or a conservative variant thereof, and a pharmaceutical carrier. Preferably, the pharmaceutical carrier is formulated for injection. Preferably, the peptide vaccine is formulated in unit dosage form. Preferably, the peptide vaccine elicits an immune response when used to inoculate an animal. In some embodiments, the peptide vaccine elicits a cellular immune response when used to inoculate an animal. In other embodiments, the peptide vaccine elicits a humoral immune response when used to inoculate an animal. The animal can be a reptile. In some embodiments, the animal is an avian. In other embodiments, the animal is a mammal. In further embodiments, the animal is a human.

The invention provides a microorganism vaccine against SARS that includes a microorganism that expresses a polypeptide or a peptide fragment of the invention, or a conservative variant thereof, and a pharmaceutical carrier. Preferably, the microorganism is attenuated. In some embodiments, the microorganism is Salmonella. In other embodiments, the microorganism is Listeria. In further embodiments, the microorganism is Listeria monocytogenes. In some embodiments, the pharmaceutical carrier is formulated for injection. In other embodiments, the pharmaceutical carrier is formulated for oral administration. Preferably, the microorganism vaccine is formulated in unit dosage form. Preferably, the microorganism vaccine elicits an immune response when used to inoculate an animal. In some embodiments, the microorganism vaccine elicits a cellular immune response when used to inoculate an animal. In other embodiments, the microorganism vaccine elicits a humoral immune response when used to inoculate an animal. The animal can be a reptile. In some embodiments, the animal is an avian. In other embodiments, the animal is a mammal. In further embodiments, the animal is a human.

The invention provides a DNA vaccine against SARS that includes a vector into which is inserted a nucleic acid segment of the invention, and a pharmaceutical carrier. The DNA vaccine may include an adjuvant. The DNA vaccine may include a myonecrotic agent. For example, the myonecrotic agent can be bupivicaine. In other embodiments, the myonecrotic agent is cardiotoxin. The vector can, for example, be a virus. In other embodiments, the vector is a bacteriophage. In further embodiments, the vector is a plasmid. The vector containing the insert can be prepared in a eukaryotic cell. However, in some embodiments, the vector containing the insert is prepared in a prokaryotic cell. For example, the vector containing the insert can be prepared in a bacterium. In some embodiments, the pharmaceutical carrier is formulated for mucosal delivery. In other embodiments, the pharmaceutical carrier is formulated for injection. Preferably, the DNA vaccine is formulated in unit dosage form. Preferably, the DNA vaccine elicits an immune response when used to inoculate an animal. In some embodiments, the DNA vaccine elicits a humoral immune response when used to inoculate an animal. In other embodiments, the DNA vaccine elicits a cellular immune response when used to inoculate an animal. The animal can be a reptile. In some embodiments, the animal is an avian. In other embodiments, the animal is a mammal. In further embodiments, the animal is a human.

The invention provides an antibody that binds to a polypeptide or peptide fragment of the invention, or a conservative variant thereof. In some embodiments, the antibody is an antigen-binding antibody fragment. In other embodiments, the antibody is a polyclonal antibody. In further embodiments, the antibody is a single-chain antibody. In other embodiments, the antibody is a monoclonal antibody. In some preferred embodiments, the antibody is a humanized antibody. The antibody may be coupled to a detectable tag. For example, the detectable tag can be a radiolabel. In some embodiments, the detectable tag is an affinity tag. In other embodiments, the detectable tag is an enzyme. In further embodiments, the detectable tag is a fluorescent protein. In some preferred embodiments, the detectable tag is a fluorescent marker. The antibody may also be coupled to a toxin.

The invention provides aptamers that bind to a polypeptide or peptide fragment of the invention, or a conservative variant thereof. The aptamer may be coupled to a detectable tag. For example, the detectable tag is a radiolabel. In some embodiments, the detectable tag is an affinity tag. In other embodiments, the detectable tag is an enzyme. In further embodiments, the detectable tag is a fluorescent protein. In some preferred embodiments, the detectable tag is a fluorescent marker. The aptamer may also be coupled to a toxin.

The invention provides a pharmaceutical composition or a kit containing an antibody, S polypeptide or aptamer of the invention and a pharmaceutical carrier. Preferably, the pharmaceutical composition is formulated for injection.

### BRIEF DESCRIPTION OF THE FIGURES

This patent or application file contains at least one drawing executed in color. Copies of this patent or patent application publication with color drawing(s) will be provided by the Office upon request and payment of the necessary fee.

FIG. 1A illustrates an agarose gel electrophoresis of a DNA construct having an insert that encodes the spike protein of the invention. Lanes from left to right: Lane 1 is a one kb DNA ladder (markers from bottom to top −0.5, 1.0, 1.6, 2.0, 3.0, 4.0); Lane 2 shows the DNA construct digested with BamHI/XbaI, resulting in the distinctive vector band (upper band) and the DNA fragment that encodes the spike protein (lower band); Lane 3 shows the DNA construct digested with HindIII which produced a smaller band and a larger band as expected due to the presence of a HindIII site in the vector and within the DNA fragment encoding the spike protein.

FIG. 1B provides a schematic diagram of a monomer of the full-length SARS-CoV S glycoprotein showing various soluble polypeptide fragments after removal of the signal sequence (residues 1-16, SEQ ID NO:60). The soluble fragments are spike protein fragments named “S” followed by numbers corresponding to the spike protein amino acids that constitute the termini of the fragment. Thus, “S756” is a soluble spike protein fragment beginning at amino acid 17 (just after the signal sequence) and ending at amino acid 756. “TM” denotes the transmembrane segment and the arrow indicates a possible cleavage site within amino acid positions 758-761 (sequence RNTR). “RBD” indicates the potential receptor-binding domain that is within amino acid positions 272-537 (SEQ ID NO: 57), likely between a residue downstream from position 303 and a residue upstream of position 537 (SEQ ID NO:61).

FIG. 2 illustrates a denaturing polyacrylamide gel electrophoresis (SDS-PAGE) of the expression of a peptide fragment of the spike protein from SARS-CoV in Escherichia coli. The peptide fragment corresponds to amino acids 17-446 of SEQ ID NO: 1. The nucleic acid segment encoding amino acids 17-446 was cloned into a pRSET vector to create pRSET-S(17-446), which was expressed in BL21DE3 cells. Numbers and arrows on the left indicate molecular weight markers in kilodaltons. The lanes contain the following polypeptides: M—molecular weight markers; lanes 1 and 2—polypeptides of control E. coli containing the pRSET vector without the nucleic acid segment encoding amino acid residues 17-446 of SEQ ID NO: 1 and without isopropylthiogalactoside (IPTG) induction; lane 3—polypeptides of control E. coli containing the pRSET vector without the nucleic acid segment encoding amino acid residues 17-446 of SEQ ID NO: 1 but with IPTG induction; lane 4—analysis of E. coli containing the pRSET vector with a nucleic acid segment encoding amino acid residues 17-446 of SEQ ID NO: 1, and with IPTG induction. The arrow on the right side indicates the position of a peptide fragment corresponding to amino acid residues 17-446 of SEQ ID NO: 1 as expressed in E. coli.

FIG. 3 illustrates a slot blot analysis of the expression of the indicated peptide fragments of the spike protein from SARS-CoV in mammalian cells. Nucleic acid segments coding for the peptide fragments were cloned into a pSecTag2B vector to express peptide fragments having the mouse k chain leader sequence at the N-terminus for secretion, and a c-Myc epitope plus a histidine tag at the C-terminus for detection and affinity purification. The nucleic acid constructs were transformed into HEK293 and VeroE6 cells. Expression of the indicated peptide fragments was examined through use of slot blot analysis with an anti-c-Myc antibody. The numbers on the left and right indicate the amino acid residues included within the detected peptide fragments. The left column represents expression of the peptide fragments in HEK293 cells. The right column represents expression of the peptide fragments in VeroE6 cells. The upper half represents samples obtained from medium in which the cells were grown (secreted proteins), and the lower half represents samples obtained from cell lysate (intracellular portion). PC is a positive control, provided by the manufacturer of the plasmid that contains PSA with a c-Myc tag at the C-terminus. NC is a negative control that contains the full length spike protein from SARS-CoV that lacks a c-Myc epitope or histidine tag.

FIG. 4A illustrates a slot blot analysis of the expression of the indicated peptide fragments from the spike protein from SARS-CoV in human 293 or Monkey VeroE6 cells. Supernatants of 293 and Vero E6 cells transfected with plasmids encoding S fragments (S276, S537, and S756) in the absence or presence of T7 polymerase expressed by recombinant vaccinia virus (VTF7.3) were transferred to nitrocellulose membranes and detected with anti-c-Myc epitope antibody. The numbers on the left and right indicate the amino acid residues included within the detected peptide fragments. PSA PC is a positive control that contains PSA with a c-Myc tag at the C-terminus. pCDNA-S NC is a negative control that contains the full length spike protein from SARS-CoV that lacks a c-Myc epitope or histidine tag. The lanes are as follows: (1) human 293 cells that were not infected with a VTF7.3 vaccinia virus, (2) human 293 cells that were infected with a VTF7.3 vaccinia virus, (3) monkey VeroE6 cells that were not infected with a VTF7.3 vaccinia virus, and (4) monkey VeroE6 cells that were infected with a VTF7.3 vaccinia virus.

FIG. 4B Supernatants from transfected cells as described above for FIG. 4A were incubated with Ni-NTA agarose beads, washed, and subjected to Western blotting with the same anti-c-Myc epitope antibody as in FIG. 4A.

FIG. 4C illustrates detection of S fragments by two rabbit polyclonal antibodies raised against peptides corresponding to sequences starting at residues 24 (D24, middle panel) and 540 (P540, right panel), respectively. The left panel shows for comparison Western blot where S537 and S756 were detected by the anti-c-Myc epitope antibody.

FIG. 5 illustrates that the full-length membrane-associated S protein is expressed on the surface of cells, as shown by flow cytometry using the rabbit polyclonal antibody P540. A nucleic acid encoding the full-length S glycoprotein was used to transfect 293 cells, which were then infected with VTF7.3. Cells were collected and incubated with P540 polyclonal antibody plus anti-rabbit secondary antibody conjugated with FITC, washed, and subjected to flow cytometry analysis. The same plasmid used to express S but without the nucleic acids for S was used to transfect cells in a control experiment denoted as negative control (NC); cells with nucleic acids encoding the full-length S glycoprotein are denoted as S.

FIGS. 6A and 6B illustrate that substantially no cleavage of the S glycoprotein occurs naturally. Western blots of supernatants from transfected 293 cells expressing S756, Se, and cell lysate of 293 cells expressing the S glycoproteins using the P540 antibody are shown. Close to background level cleavage of S and Se was observed. FIG. 6A shows a Western blot of samples kept for three days at 4° C. before analysis to monitor the effect of nonspecific protease activity on the cleavage pattern. In contrast, FIG. 6B shows blots with samples used immediately after preparation.

FIG. 7A-C shows that cell fusion is mediated by the S glycoprotein. A pCDNA3-based plasmid without S insert was used as plasmid control, and fusion between S-expressing cells with ACE2-ecto expressing cells was used as negative control. The pCDNA3-ACE2-ecto construct expresses just the ACE2 soluble ecto domain tagged with C9 peptide. FIG. 7A illustrates that there was no syncytium formation between 293T cells transfected with pSecTag2B-S and pCDNA3-ACE2-Ecto. In contrast, FIG. 7B illustrates syncytium formation between 293T cells transfected with pSecTag2B-S and pCDNA3-ACE2, respectively. FIG. 7C graphically illustrates cell fusion as measured by a reporter gene-based assay. As shown, S glycoprotein expressed in both pCDNA3 and pSecTag2B vectors can be detected in a β-gal reporter gene-based cell-cell fusion assay.

FIG. 8A-C shows that the S glycoprotein receptor-binding domain (RBD) is localized between residues 272 and 537. FIG. 8A illustrates binding of two different S soluble fragments (S537 and S756) to 293 and Vero E6 cells. FIG. 8B illustrates binding of various S fragments to Vero E6 cells. The background OD₄₀₅ measured for the negative control was subtracted from the OD₄₀₅ values of each S fragment. The resulting OD₄₀₅ for each fragment was then presented as a percentage of the OD₄₀₅ for S537. FIG. 8C illustrates which S polypeptide fragments interact with purified soluble ACE2 as measured by ELISA. In all experiments, the negative control (NC) represents sample processed exactly the same way as the others except that the plasmid used for transfection did not encode any protein. Data shown here represent at least three independent experiments. OD₄₀₅ for all samples is presented as percentages of the OD₄₀₅ for S537.

FIG. 9A-D illustrates that dimerization occurs between the N terminal fragments of the SARS-CoV S glycoprotein as demonstrated by co-immunoprecipitation and cross-linking. All N-terminal fragments except the smallest fragment (S317-517) containing the receptor binding domain were coimmunoprecipiated with S756 by the P540 antibody. The P540 antibody is a rabbit polyclonal antibody that was developed against a peptide containing residues 540-555 of the S glycoprotein and it binds the S756 polypeptide but not the N-terminal fragments.

In FIG. 9A, plasmids encoding N-terminal fragments (denoted by the number of the ending amino acid residue or the number of the starting and ending residue) were used to transfect 293T cells alone (left six lanes) or in combination (right four lanes) with a S756-encoding plasmid. These cells were then infected with the vaccinia virus VTF7.3. After incubation, the culture medium was collected and subjected to Western blot analysis using a mouse anti-c-Myc epitope antibody that recognizes all fragments.

FIG. 9B shows that all N-terminal S fragments, except the smallest fragment (S317-517) that contained the receptor binding domain, were coimmunoprecipiated with S756 by the P540 antibody. The same medium samples used in FIG. 9A were subjected first to immunoprecipitation with the P540 polyclonal antibody that recognizes only S756. These immunoprecipitates were then subjected to Western blot analysis using the anti-c-Myc epitope antibody to confirm that the N-terminal fragments coimmunoprecipitated.

FIG. 9C shows that a new band with a molecular weight corresponding to a dimer forms in the presence and absence of DTT. To rule out the possibility of nonspecific disulfide bond formation that may lead to coimmunoprecipitation, DTT was included in one of the coimmunoprecipitation experiment. DTT had no effect on either immunoprecipitation or coimmunoprecipitation of secreted S756 (left lanes) or S756+S276 (right lanes). Medium samples containing secreted S756 (left lanes) or S756+S276 (right lanes) fragments were subjected to immunoprecipitation with P540 in the presence or absence of 2 mM DTT.

FIG. 9D illustrates the size of the S polypeptide oligomers. The S537 fragment was cross-linked with BS³ (Pierce, Rockford, Ill.) as described in the Examples and a Western blot was prepared after SDS-PAGE separation and the anti-c-Myc antibody was used for detection of the S537 monomer and its oligomers. As shown in the right lane of FIG. 9D, a new band appears when the crosslinking reagent is added. The new band had a molecular weight corresponding to a dimer but not of higher order oligomers.

FIG. 10A illustrates dimerization of the N terminal fragment S537 as detected by size-exclusion chromatography. The elution profiles of S537 and S317-517 are shown with arrows and numbers indicating the position and molecular weight at which standard calibration proteins were eluted.

FIG. 10B provides western blots of fractions collected for S537 and S317-517 by using an anti-c-Myc epitope antibody.

FIG. 11A-B illustrates that the extreme N terminal domain is required for the S glycoprotein mediated cell-cell fusion. FIG. 11A provides a schematic representation of the S glycoprotein deletion mutants and a summary of the data from a cell-cell fusion assay where RBD denotes the approximate position of the receptor binding domain. The presence of signal due to fusion is denoted by a plus (+) and lack of measurable signal above background levels by a minus (−). Only wild type polypeptides with amino acids 17-1255 had fusion activity. Neither of the deletion mutants having amino acids 103-1255 (Del1) or 311-1255 (Del2) had fusion activity. FIG. 11B shows the levels of expression of full length and deletion mutants of the S glycoprotein as measured by Western analysis. Equal amount of cell lysates were loaded for each sample and the rabbit polyclonal antibody P540 was used for detection. FIG. 11C illustrates that the full length S glycoprotein and the Del1 and Del2 deletion mutants are expressed on the cell surface as measured by flow cytometry. The level of surface expression was low although the negative control where the cells were transfected with an empty plasmid was clearly distinguishable to the left of the other three curves.

FIG. 12A-B illustrates that dimeric S1 binds more efficiently to the receptor ACE2 than monovalent fragments containing the receptor binding domain. FIG. 12A shows the relative levels of expression of different S fragments as detected by ELISA using 200 μl of culture supernatants from cells transfected with S276, S319-518 and S537 constructs. Anti-His and anti-c-Myc epitope antibodies were used in a sandwich ELISA to detect the levels of secreted tagged S proteins. FIG. 12B shows the level of binding by S fragments to ACE2 as measured by ELISA. The tagged ACE2 was bound to plates by an anti-C9 antibody that had been previously coated on the plates. The supernatants from cell cultures where the cells were transfected with various S proteins were mixed and incubated in ELISA plates either with (hatched bars) or without (open bars) anti-c-Myc antibody. The highest level of expression or binding is assumed to be 100%. As shown the S537 fragment with both the N-terminal dimerization domain and the receptor binding domain, binds ACE2 more efficiently than does the S319-518 fragment that has only the receptor binding domain.

FIG. 13A-B illustrates that the soluble S ectodomain is trimeric under the conditions of size-exclusion chromatography. In FIG. 13A, purified Se was run on a gel filtration column that was calibrated by using proteins with known molecular weight. BSA in equal amount was included as an internal control. In FIG. 13B, different fractions were collected from the gel filtration column and analyzed by Western blot. Two bands S polypeptide are detected in some fractions that contain Se fragments of the indicated molecular weights, representing the Se fragment alone (lower band) and its aggregates (upper band).

FIG. 14A illustrates that a DNA vaccine of the invention can elicit very high titer anti-SARS-CoV sera in mice. Mice 1A-5A were immunized with DNA encoding the S319-518 fragment that contains the spike protein receptor binding domain (RBD). Mice 1B-5B were immunized with RBD-encoding DNA (the S319-518 fragment) fused to a nucleic acid encoding an Fc fragment. Mice 1C-3C received plasmid only (no S fragment DNA). Anti-sera were collected and tested via ELISA to ascertain the titer of the different isolates. In FIG. 14A, the first number denotes an individual mouse, the letter denotes the respective immunization group, and the last number denotes the dilution used. Anti-sera were diluted by factors of 50, 250, 1250 and 7250, as shown on the x-axis of the bar graph. These data indicate that immunization with DNA encoding the receptor binding domain of the S protein induces a strong immune response against SARS-CoV.

FIG. 14B illustrates that anti-sera from mice immunized with RBD-encoding DNA can prevent S-mediated cell fusion. Cells (293T) were incubated with anti-sera from mice immunized with DNA encoding a spike protein receptor binding domain polypeptide (S319-518) fragment and then the cell suspension was mixed with cells expressing S protein. Fusion was measured as described in Example 20 (see also, Xiao et al. BBRC 2003). The percentage (where 1=100%) of activity for each fusion reaction is plotted on the y-axis, where the percentage of the fusion without any inhibition was designated as 100%. PC denotes positive control where no serum was added. For mice sera #1 to #2 in each group, serum dilution factors of 10 (designated 0.1), 100 (designated 0.01), and 1000 (designated (0.001) were used. For mice sera #3-#5 in groups A and B, and #3 in the control group, dilution factors of 20 (designated 0.05) and 100 (designated 0.01) were used. These data indicate that immunization with DNA encoding the receptor binding domain of the S protein could prevent SARS-CoV infection.

FIG. 15 illustrates that soluble S glycoprotein fragments inhibit S-mediated cell fusion. 10 ug/ml of various S fragments were incubated with ACE2-expressing cells first for 10 min at room temperature. The ACE2-expressing cells were then mixed with S expressing cells and the fusion assay was carried out as described in the Examples. The Y-axis is the OD₅₉₅ for each sample after the background noise was subtracted. Numbers of each construct represent the starting and ending residues of the respective polypeptide.

### DETAILED DESCRIPTION OF THE INVENTION

SARS represents an important public health concern. Methods to diagnose and treat persons who are infected with SARS-CoV provide the opportunity to either prevent or control further spread of infection by SARS-CoV. These methods are especially important due to the ability of SARS-CoV to infect persons through an airborne route. The present invention provides nucleic acids that encode segments of the amino acid sequence of the spike protein of SARS-CoV. The present invention also provides polypeptides that correspond in amino acid sequence to segments of the amino acid sequence of the spike protein of SARS-CoV. The invention also provides peptide fragments and conservative variants of the spike protein of SARS-CoV, in addition to coupled proteins and peptidomimetics that have portions which correspond in amino acid sequence to the spike protein.

The spike protein is important because it is present on the outside of intact SARS-CoV. Thus, it presents a target that can be used to inhibit or eliminate an intact virus before the virus has an opportunity to infect a cell.

The nucleic acids and polypeptides of the invention offer advantages over the full length spike protein because the nucleic acids are easy to produce and the polypeptides of the invention are produced in large amounts in soluble form. The polypeptides of the invention offer additional advantages over the native spike protein because they can be made to have increased resistant to degradation when administered to an animal. The polypeptides of the invention can also be formulated to increase their antigenicity to make them more efficient antigens to elicit an immune response when administered to an animal, such as a human.

Accordingly, the invention provides nucleic acids and polypeptide antigens that may be used to formulate vaccines and immune compositions that can be used to immunize and treat persons who are infected with SARS-CoV. In addition, the invention provides antibodies that bind to the spike protein of SARS-CoV which may be used to diagnose, immunize, and treat persons infected with SARS-CoV.

Definitions:

An “adjuvant” is generally defined as a substance that nonspecifically enhances the immune response to an antigen. A variety of adjuvants may be employed with the immunopeptides and immunofragopeptides of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

An “animal” refers to an organism that can mount an immune response upon antigenic challenge. For example, reptiles, avians, and mammals are able to produce antibodies in response to an antigenic challenge. Antibodies raised in non-human organisms are thought to be useful in diagnostic assays to reduce or eliminate cross-reactivity.

An “aptamer” is a peptide, polypeptide or nucleic acid (RNA or DNA) that binds to a polypeptide or peptide fragment of the invention.

A “carrier protein” refers to a polypeptide that can be coupled with a polypeptide or a peptide fragment of the invention to form a coupled protein. A carrier protein may be coupled to a polypeptide or peptide fragment in order to increase the solubility or the immunogenicity of the polypeptide or peptide fragment. A carrier protein may also be coupled to a polypeptide or peptide fragment to provide a tag which provides for separation or detection of the coupled protein. For example, biotin may be used as a carrier protein that is coupled to a polypeptide or peptide fragment to create a coupled protein which can then be isolated through interaction with avidin, or detected through use of a fluorescently tagged avidin. In another example, a carrier protein that is bound by an antibody can be coupled to a polypeptide or peptide fragment to create a coupled protein that is bound by the antibody which binds to the carrier protein of the coupled protein.

The invention encompasses isolated or substantially purified nucleic acids, peptides, polypeptides or proteins. In the context of the present invention, an “isolated” nucleic acid, DNA or RNA molecule or an “isolated” polypeptide is a nucleic acid, DNA molecule, RNA molecule, or polypeptide that exists apart from its native environment and is therefore not a product of nature. An isolated nucleic acid, DNA molecule, RNA molecule or polypeptide may exist in a purified form or may exist in a non-native environment such as, for example, a transgenic host cell. A “purified” nucleic acid molecule, peptide, polypeptide or protein, or a fragment thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein, peptide or polypeptide that is substantially free of cellular material includes preparations of protein, peptide or polypeptide having less than about 30%, 20%, 10%, or 5% (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals.

The terms polypeptide, peptide and protein are used interchangeably herein.

A peptide or polypeptide “fragment” as used herein refers to a less than full length peptide, polypeptide or protein. For example, a peptide or polypeptide fragment can have is at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length. Preferably the peptide fragment can elicit an immune response when used to inoculate an animal. A peptide fragment may be used to elicit an immune response by inoculating an animal with a peptide fragment in combination with an adjuvant, a peptide fragment that is coupled to an adjuvant, or a peptide fragment that is coupled to arsanilic acid, sulfanilic acid, an acetyl group, or a picryl group. A peptide fragment can include a non-amide bond, and can be a peptidomimetic.

The term “soluble” as used herein refers to the ability of a polypeptide to be solvated in an aqueous solution. For example, a soluble peptide can be mixed with an aqueous medium such that at least a detectable portion of the peptide is present in the aqueous medium. The peptide may be detected through use of common techniques, such as absorbance of light, fluorescence, the ability to bind dyes, the ability to reduce silver ions, and the like.

The term “specifically binds” refers to an antibody that binds to a single epitope, but which does not bind to more than one epitope. Accordingly, an antibody that specifically binds to a polypeptide will bind to an epitope that present on the polypeptide, but which is not present on other polypeptides.

I. Polypeptides, Peptide Fragments, Coupled Proteins, Immunopeptides, and Peptidomimetics of the Invention

The invention provides a polypeptide which has an amino acid sequence that corresponds to the amino acid sequence of the spike protein from the virus (SARS-CoV) that is etiologically linked to severe acute respiratory syndrome (SARS). A representative amino acid sequence is provided by SEQ ID NO: 1, whose sequence is provided below for easy reference.

| | |
|---|---|
| 1 | MFIFLLFLTL TSGSDLDRCT TFDDVQAPNY TQHTSSMRGV |
| 41 | YYPDEIFRSD TLYLTQDLFL PFYSNVTGFH TINHTFGNPV |
| 81 | IPFKDGIYFA ATEKSNVVRG WVFGSTMNNK SQSVIIINNS |
| 121 | TNVVIRACNF ELCDNPFFAV SKPMGTQTHT MIFDNAFNCT |
| 161 | FEYISDAFSL DVSEKSGNFK HLREFVFKNK DGFLYVYKGY |
| 201 | QPIDVVRDLP SGFNTLKPIF KLPLGINITN FRAILTAFSP |
| 241 | AQDTWGTSAA AYFVGYLKPT TFMLKYDENG TITDAVDCSQ |
| 281 | NPLAELKCSV KSFEIDKGIY QTSNFRVVPS GDVVRFPNIT |
| 321 | NLCPFGEVFN ATKFPSVYAW ERKKISNCVA DYSVLYNSTF |
| 361 | FSTFKCYGVS ATKLNDLCFS NVYADSFVVK GDDVRQIAPG |
| 401 | QTGVIADYNY KLPDDFMGCV LAWNTRNIDA TSTGNYNYKY |
| 441 | RYLRHGKLRP FERDISNVPF SPDGKPCTPP ALNGYWPLND |
| 481 | YGFYTTTGIG YQPYRVVVLS FELLNAPATV CGPKLSTDLI |
| 521 | KNQCVNFNFN GLTGTGVLTP SSKRFQPFQQ FGRDVSDFTD |
| 561 | SVRDPKTSEI LDISPCAFGG VSVITPGTNA SSEVAVLYQD |
| 601 | VNCTDVSTAI HADQLTPAWR IYSTGNNVFQ TQAGCLTGAE |
| 641 | HVDTSYECDI PIGAGICASY HTVSLLRSTS QKSIVAYTMS |
| 681 | LGADSSIAYS NNTIAIPTNF SISITTEVMP VSMAKTSVDC |
| 721 | NMYICGDSTE CANLLLQYGS FCTQLNRALS GIAAEQDRNT |
| 761 | REVFAQVKQM YKTPTLKYFG GFNFSQILPD PLKPTKRSFI |
| 801 | EDLLFNKVTL ADAGFMKQYG ECLGDINARD LICAQKFNGL |
| 841 | TVLPPLLTDD MIAAYTAALV SGTATAGWTF GAGAALQIPF |
| 881 | AMQMAYRFNG IGVTQNVLYE NQKQIANQFN KAISQIQESL |
| 921 | TTTSTALGKL QDVVNQNAQA LNTLVKQLSS NFGAISSVLN |
| 961 | DILSRLDKVE AEVQIDRLIT GRLQSLQTYV TQQLIRAAEI |
| 1001 | RASANLAATK MSECVLGQSK RVDFCGKGYH LMSFPQAAPH |
| 1041 | GVVFLHVTYV PSQERNFTTA PAICHEGKAY FPREGVFVFN |
| 1081 | GTSWFITQRN FFSPQIITTD NTFVSGNCDV VIGITNNTVY |
| 1121 | DPLQPELDSF KEELDKYFKN HTSPDVDLGD ISGINASVVN |
| 1161 | IQKEIDRLNE VAKNLNESLI DLQELGKYEQ YIKWPWYVWL |
| 1201 | GFIAGLIAIV MVTILLCCMT SCCSCLKGAC SCGSCCKFDE |
| 1241 | DDSEPVLKGV KLHYT |

The invention also provides peptide fragments which have amino acid sequences that correspond to a fragment of the spike protein from the virus (SARS-CoV) that is etiologically linked to severe acute respiratory syndrome (SARS). Such amino acid sequences include those represented by SEQ ID NOs: 13, 14, 15, 20-59, and 61-63. The peptide fragments of SEQ ID NO: 1 can also be three or more amino acids in length, and produce an immune response when used to immunize an animal. These peptide fragments are exemplified by those that are three amino acids in length, or single amino acid units of greater length, such as 4, 5, 6, 7, 8, 9, 10 amino acids in length, and an amino acid sequence that lacks one amino acid from the amino acid sequence corresponding to SEQ ID NO: 1.

The invention also provides coupled proteins having a carrier protein coupled to a polypeptide or peptide fragment of the invention. The carrier protein may be used to increase the solubility of the coupled protein. The carrier protein may also be used to increase the immunogenicity of the coupled protein to increase production of antibodies that bind to the polypeptide or peptide fragment of the invention. The carrier protein may also be used to provide for the separation or detection of a coupled protein. Accordingly, a coupled protein can be detected or isolated by interaction with other components that bind to the carrier protein portion of the coupled protein. For example, a coupled protein having avidin as a carrier protein can be detected or separated with biotin through use of known methods. Numerous carrier proteins may be used to create coupled proteins of the invention. Examples of such carrier proteins include, keyhole limpet hemacyanin, bovine serum albumin, ovalbumin, mouse serum albumin, rabbit serum albumin, and the like. A carrier protein may be coupled to a polypeptide or peptide fragment of the invention by creation of a fusion protein through use of recombinant methods. A carrier protein may also be coupled to a polypeptide or peptide fragment of the invention through use of chemical linking methods, or through use of a chemical linker. Such coupling methods are known in the art and have been described. Harlow et al., Antibodies: A Laboratory Manual, page 319 (Cold Spring Harbor Pub. 1988); Taylor, Protein Immobilization, Marcel Dekker, Inc., New York, (1991).

The invention provides immunopeptides having a polypeptide or a peptide fragment of the invention coupled to arsanilic acid, sulfanilic acid, an acetyl group, or a picryl group. Methods to couple such groups to peptides are known and have been reported. Weigle, J. Exp. Med., 116:913-928 (1962); Weigle, J. Exp. Med., 122:1049-1062 (1965); Weigle, J. Exp. Med., 121:289-308 (1965).

The polypeptides and peptide fragments of the invention may be in glycosylated form, or in unglycosylated form. A polypeptide or peptide fragment of the invention may be soluble or insoluble in aqueous solution. The polypeptides and peptide fragments of the invention may be conservative variants. A conservative variant is a polypeptide or peptide fragment derived from a full-length polypeptide, such as that exemplified by SEQ ID NO: 1, by deletion (so-called truncation), addition, or subtraction of one or more amino acids to the N-terminal and/or C-terminal end of the full-length polypeptide; deletion, addition or subtraction of one or more amino acids at one or more sites in the full-length polypeptide. Such variants may result from, for example, genetic polymorphism or from human manipulation. Methods for such manipulations are generally known in the art. For example, amino acid sequence variants of SEQ ID NO: 1 can be prepared by mutagenesis of DNA encoding the polypeptide. Methods for mutagenesis and nucleotide sequence alterations are well known in the art. See, for example, Kunkel, Proc. Natl. Acad. Sci. USA, 82, 488 (1985); Kunkel et al., Methods in Enzymol., 154:367 (1987); U.S. Pat. No. 4,873,192; Walker and Gaastra, eds., Techniques in Molecular Biology, MacMillan Publishing Company, New York (1983) and the references cited therein. Guidance as to appropriate amino acid substitutions may be found in the model of Dayhoff et al., Atlas of Protein Sequence and Structure, Natl. Biomed. Res. Found., Washington, C.D. (1978), herein incorporated by reference. Conservative substitutions, such as exchanging one amino acid with another having similar properties, are preferred. For example, substitution of a hydrophobic amino acid for another, or substitution of a hydrophilic amino acid for another. Routine screening assays can be used to determine if a substituted polypeptide or peptide fragment derived from SEQ ID NO: 1 produces an immune response when administered to a mammal. Examples of such screening assays are well known in the art and include enzyme linked immunosorbant assays, radioimmuno assays, chromium release assays, and the like. Such assays have been described. Harlow et al., Antibodies: A Laboratory Manual, page 319 (Cold Spring Harbor Pub. 1988).

The invention provides peptidomimetics of the polypeptides and peptide fragments of the invention. A peptidomimetic describes a peptide analog, such as those commonly used in the pharmaceutical industry as non-peptide drugs, with properties analogous to those of the template peptide. (Fauchere, J., Adv. Drug Res., 15: 29 (1986) and Evans et al., J. Med. Chem., 30:1229 (1987)). Peptidomimetics are structurally similar to polypeptides or peptide fragments having peptide bonds, but have one or more peptide linkages optionally replaced by a linkage such as, —CH₂NH—, —CH₂S—, —CH₂—CH₂—, —CH═CH— (cis and trans), —COCH₂—, —CH(OH)CH₂—, and —CH₂SO—, by methods known in the art. Advantages of peptide mimetics over natural polypeptide embodiments may include more economical production, greater chemical stability, altered specificity and enhanced pharmacological properties such as half-life, absorption, potency and efficacy.

The polypeptides, peptide fragments, coupled proteins, and peptidomimetics of the invention can be modified for in vivo use by the addition, at the amino-terminus and/or the carboxyl-terminus, of a blocking agent to decrease degradation in vivo. This can be useful in those situations in which the polypeptide termini tend to be degraded by proteases prior to cellular uptake. Such blocking agents can include, without limitation, additional related or unrelated peptide sequences that can be attached to the amino and/or carboxyl terminal residues of the polypeptide, peptide fragment, coupled protein, and peptidomimetic to be administered. This can be done either chemically during the synthesis of the polypeptide, peptide fragment, or coupled protein, or by recombinant DNA technology by methods familiar to artisans of average skill. Alternatively, blocking agents such as pyroglutamic acid, or other molecules known in the art, can be attached to the amino and/or carboxyl terminal residues, or the amino group at the amino terminus or carboxyl group at the carboxyl terminus can be replaced with a different moiety. Accordingly, the invention provides polypeptides and peptide fragments that are amino-terminally and carboxyl-terminally blocked.

The ability of a polypeptide or peptide fragment of the invention to produce an immune response may be tested through numerous art recognized methods. For example, for their ability to induce antibody production, or to stimulate a cytotoxic T-lymphocyte response.

The polypeptides and peptide fragments of the invention may be used within screening assays to identify or isolate antibodies that bind to the polypeptides or peptide fragments of the invention, or the spike protein from SARS-CoV. For example, the polypeptides or peptide fragments may be used in phage display assays to isolate antibodies that bind to the polypeptides or peptide fragments. In another example, the polypeptides or peptide fragments of the invention may be bound to a solid support to which antibodies are contacted such that antibodies which bind to the polypeptides or peptide fragments become immobilized on the solid support. These antibodies can be later eluted from the solid support. The polypeptides and peptide fragments of the invention may be used to isolate antibodies according to many other methods known in the art.

Expression systems that may be used for small or large scale production of the, coupled proteins, polypeptides or peptide fragments of the invention include, but are not limited to, cells or microorganisms that are transformed with a recombinant nucleic acid construct that contains a nucleic acid segment of the invention. Examples of recombinant nucleic acid constructs may include bacteriophage DNA, plasmid DNA, cosmid DNA, or viral expression vectors. Examples of cells and microorganisms that may be transformed include bacteria (for example, E. coli or B. subtilis); yeast (for example, Saccharomyces and Pichia); insect cell systems (for example, baculovirus); plant cell systems; or mammalian cell systems (for example, COS, CHO, BHK, 293, VERO, HeLa, MDCK, W138, and NIH 3T3 cells). Also useful as host cells are primary or secondary cells obtained directly from a mammal that are transfected with a plasmid vector or infected with a viral vector. Examples of suitable expression vectors include, without limitation, plasmids and viral vectors such as herpes viruses, retroviruses, vaccinia viruses, attenuated vaccinia viruses, canary pox viruses, adenoviruses, adeno-associated viruses, lentiviruses and herpes viruses, among others. Synthetic methods may also be used to produce polypeptides and peptide fragments of the invention. Such methods are known and have been reported. Merrifield, Science, 85:2149 (1963).

II. Nucleic Acid Segments, Expression Cassettes, and Nucleic Acid Constructs of the Invention

The present invention provides isolated nucleic acid segments that encode the polypeptides, peptide fragments, and coupled proteins of the invention. The nucleic acid segments of the invention also include segments that encode for the same amino acids due to the degeneracy of the genetic code. For example, the amino acid threonine is encoded by ACU, ACC, ACA and ACG and is therefore degenerate. It is intended that the invention includes all variations of the polynucleotide segments that encode for the same amino acids. Such mutations are known in the art (Watson et al, Molecular Biology of the Gene, Benjamin Cummings 1987). Mutations also include alteration of a nucleic acid segment to encode for conservative amino acid changes, for example, the substitution of leucine for isoleucine and so forth. Such mutations are also known in the art. Thus, the genes and nucleotide sequences of the invention include both the naturally occurring sequences as well as mutant forms.

The nucleic acid segments of the invention may be contained within a vector. A vector may include, but is not limited to, any plasmid, phagemid, F-factor, virus, cosmid, or phage in double or single stranded linear or circular form which may or may not be self transmissible or mobilizable. The vector can also transform a prokaryotic or eukaryotic host either by integration into the cellular genome or exist extra-chromosomally (e.g. autonomous replicating plasmid with an origin of replication).

Preferably the nucleic acid segment in the vector is under the control of, and operably linked to, an appropriate promoter or other regulatory elements for transcription in vitro or in a host cell, such as a eukaryotic cell, or a microbe, e.g. bacteria. The vector may be a shuttle vector that functions in multiple hosts. The vector may also be a cloning vector that typically contains one or a small number of restriction endonuclease recognition sites at which foreign DNA sequences can be inserted in a determinable fashion. Such insertion can occur without loss of essential biological function of the cloning vector. A cloning vector may also contain a marker gene that is suitable for use in the identification and selection of cells transformed with the cloning vector. Examples of marker genes are tetracycline resistance or ampicillin resistance. Many cloning vectors are commercially available (Stratagene, New England Biolabs, Clonetech).

The nucleic acid segments of the invention may also be inserted into an expression vector. Typically an expression vector contains prokaryotic DNA elements coding for a bacterial replication origin and an antibiotic resistance gene to provide for the amplification and selection of the expression vector in a bacterial host; regulatory elements that control initiation of transcription such as a promoter; and DNA elements that control the processing of transcripts such as introns, or a transcription termination/polyadenylation sequence.

Methods to introduce nucleic acid segment into a vector are available in the art (Sambrook et al., Molecular Cloning: A Laboratory Manual, 3rd edition, Cold Spring Harbor Press, Cold Spring Harbor, N.Y. (2001)). Briefly, a vector into which a nucleic acid segment is to be inserted is treated with one or more restriction enzymes (restriction endonuclease) to produce a linearized vector having a blunt end, a “sticky” end with a 5′ or a 3′ overhang, or any combination of the above. The vector may also be treated with a restriction enzyme and subsequently treated with another modifying enzyme, such as a polymerase, an exonuclease, a phosphatase or a kinase, to create a linearized vector that has characteristics useful for ligation of a nucleic acid segment into the vector. The nucleic acid segment that is to be inserted into the vector is treated with one or more restriction enzymes to create a linearized segment having a blunt end, a “sticky” end with a 5′ or a 3′ overhang, or any combination of the above. The nucleic acid segment may also be treated with a restriction enzyme and subsequently treated with another DNA modifying enzyme. Such DNA modifying enzymes include, but are not limited to, polymerase, exonuclease, phosphatase or a kinase, to create a nucleic acid segment that has characteristics useful for ligation of a nucleic acid segment into the vector.

The treated vector and nucleic acid segment are then ligated together to form a construct containing a nucleic acid segment according to methods available in the art (Sambrook et al., Molecular Cloning: A Laboratory Manual, 3rd edition, Cold Spring Harbor Press, Cold Spring Harbor, N.Y. (2001)). Briefly, the treated nucleic acid fragment and the treated vector are combined in the presence of a suitable buffer and ligase. The mixture is then incubated under appropriate conditions to allow the ligase to ligate the nucleic acid fragment into the vector.

The invention also provides an expression cassette which contains a nucleic acid sequence capable of directing expression of a particular nucleic acid segment of the invention, such as SEQ ID NO: 2, either in vitro or in a host cell. Also, a nucleic acid segment of the invention may be inserted into the expression cassette such that an anti-sense message is produced. The expression cassette is an isolatable unit such that the expression cassette may be in linear form and functional for in vitro transcription and translation assays. The materials and procedures to conduct these assays are commercially available from Promega Corp. (Madison, Wis.). For example, an in vitro transcript may be produced by placing a nucleic acid sequence under the control of a T7 promoter and then using T7 RNA polymerase to produce an in vitro transcript. This transcript may then be translated in vitro through use of a rabbit reticulocyte lysate. Alternatively, the expression cassette can be incorporated into a vector allowing for replication and amplification of the expression cassette within a host cell or also in vitro transcription and translation of a nucleic acid segment.

Such an expression cassette may contain one or a plurality of restriction sites allowing for placement of the nucleic acid segment under the regulation of a regulatory sequence. The expression cassette can also contain a termination signal operably linked to the nucleic acid segment as well as regulatory sequences required for proper translation of the nucleic acid segment. The expression cassette containing the nucleic acid segment may be chimeric, meaning that at least one of its components is heterologous with respect to at least one of its other components. The expression cassette may also be one which is naturally occurring but has been obtained in a recombinant form useful for heterologous expression. Expression of the nucleic acid segment in the expression cassette may be under the control of a constitutive promoter or an inducible promoter which initiates transcription only when the host cell is exposed to some particular external stimulus.

The expression cassette may include in the 5′-3′ direction of transcription, a transcriptional and translational initiation region, a nucleic acid segment and a transcriptional and translational termination region functional in vivo and/or in vitro. The termination region may be native with the transcriptional initiation region, may be native with the nucleic acid segment, or may be derived from another source.

The regulatory sequence can be a polynucleotide sequence located upstream (5′ non-coding sequences), within, or downstream (3′ non-coding sequences) of a coding sequence, and which influences the transcription, RNA processing or stability, or translation of the associated coding sequence. Regulatory sequences can include, but are not limited to, enhancers, promoters, repressor binding sites, translation leader sequences, introns, and polyadenylation signal sequences. They may include natural and synthetic sequences as well as sequences which may be a combination of synthetic and natural sequences. While regulatory sequences are not limited to promoters, some useful regulatory sequences include constitutive promoters, inducible promoters, regulated promoters, tissue-specific promoters, viral promoters and synthetic promoters.

A promoter is a nucleotide sequence which controls the expression of the coding sequence by providing the recognition for RNA polymerase and other factors required for proper transcription. A promoter includes a minimal promoter, consisting only of all basal elements needed for transcription initiation, such as a TATA-box and/or initiator that is a short DNA sequence comprised of a TATA-box and other sequences that serve to specify the site of transcription initiation, to which regulatory elements are added for control of expression. A promoter may be derived entirely from a native gene, or be composed of different elements derived from different promoters found in nature, or even be comprised of synthetic DNA segments. A promoter may contain DNA sequences that are involved in the binding of protein factors which control the effectiveness of transcription initiation in response to physiological or developmental conditions.

The invention also provides a construct containing a vector and an expression cassette. The vector may be selected from, but not limited to, any vector previously described. Into this vector may be inserted an expression cassette through methods known in the art and previously described (Sambrook et al., Molecular Cloning: A Laboratory Manual, 3rd edition, Cold Spring Harbor Press, Cold Spring Harbor, N.Y. (2001)). In one embodiment, the regulatory sequences of the expression cassette may be derived from a source other than the vector into which the expression cassette is inserted. In another embodiment, a construct containing a vector and an expression cassette is formed upon insertion of a nucleic acid segment of the invention into a vector that itself contains regulatory sequences. Thus, an expression cassette is formed upon insertion of the nucleic acid segment into the vector. Vectors containing regulatory sequences are available commercially and methods for their use are known in the art (Clonetech, Promega, Stratagene).

III. Immune Compositions and Vaccines of the Invention

The invention provides immune compositions and vaccines that can be used to produce an immune response against the virus that is etiologically linked to severe acute respiratory syndrome when administered to an animal. The immune response may be a humoral immune response or a cellular immune response.

An immune composition of the invention can include an adjuvant and a nucleic acid, polypeptide, peptide fragment, a peptidomimetic, a coupled protein, an immunopeptide of the invention, or any combination thereof. An immune composition can contain an adjuvant that is not chemically linked to a polypeptide, peptide fragment, a peptidomimetic, a coupled protein, or an immunopeptide of the invention. An immune composition can contain an adjuvant that is chemically linked to a polypeptide, peptide fragment, a peptidomimetic, a coupled protein, or an immunopeptide of the invention. An immune composition of the invention can also include a pharmaceutically acceptable diluent or carrier.

An immune composition may be manufactured conventionally. In particular, a nucleic acid, polypeptide, peptide fragment, peptidomimetic, coupled protein, immunopeptide, or any combination thereof that is contained in the composition may be combined with a pharmaceutically acceptable diluent or carrier. Examples of pharmaceutically acceptable diluent or carriers include water or a saline solution, such as phosphate-buffered saline (PBS). In general, the pharmaceutically acceptable diluent or carrier is selected on the basis of the mode and route of administration and of standard pharmaceutical practices. Pharmaceutically acceptable diluents and carriers as well as all that is necessary for their use in pharmaceutical compositions are described in Remington's Pharmaceutical Sciences, a standard reference text in this field.

Immune compositions may contain adjuvants as disclosed herein and as known in the art. Aluminum compounds may be used as adjuvants. Such aluminum compounds include, aluminum hydroxide, aluminum phosphate, aluminum hydroxyphosphate, and the like. The nucleic acid, polypeptide, peptide fragment, peptidomimetic, coupled protein, immunopeptide, or any combination thereof may be absorbed or precipitated on an aluminum compound according to standard methods. Other adjuvants include polyphosphazene (WO 95/2415), DC-chol (3-beta-[N-(N′,N′-dimethylaminomethane) carbamoyl) cholesterol] (U.S. Pat. No. 5,283,185 and WO 96/14831), QS-21 (WO 88/9336) and RIBI from ImmunoChem (Hamilton, Mont.). Immunostimulatory oligonucleotides containing unmethylated CpG dinucleotides (“CpG”) are known in the art as being adjuvants when administered by both systemic and mucosal routes (WO 96/02555, EP 468520, Davis et al., J. Immunol., 160:870 (1998); McCluskie and Davis, J. Immunol., 161:4463 (1998). CpG when formulated into immune compositions or vaccines, is generally administered in free solution together with free antigen (WO 96/02555; McCluskie and Davis, J. Immunol., 161:4463 (1998)) orcovalently conjugated to an antigen (PCT Publication No. WO 98/16247), or formulated with a carrier such as aluminum hydroxide. (Brazolot-Millan et al., Proc. Natl. Acad. Sci., 95:15553 (1998)).

The invention also provides vaccines that include a nucleic acid, polypeptide, a peptide fragment, a peptidomimetic, a coupled protein, an immunopeptide of the invention, a nucleic or any combination thereof. Such vaccines can be formulated as described herein or as known in the vaccine arts. For example, a viral vaccine may be created that expresses a polypeptide, a peptide fragment, or a coupled protein of the invention according to methods known in the art. Examples of viral vectors that may be used include, adenoviruses, herpes viruses, vaccinia viruses, canarypox viruses, and the like. Vaccines can also be formulated as a liposome. Such formulations are known to those skilled in the art. Liposomes: A Practical Approach. RRC New Ed, IRL press (1990).

The invention also provides nucleic acid based vaccines that express a polypeptide, a peptide fragment, or a coupled protein of the invention. For example, a nucleic acid vaccine can express a polypeptide having SEQ ID NO: 1, 13, 14, 15, 20-59, 61-63 or a fragment of SEQ ID NO: 1. Inoculation of an animal with a nucleic acid construct that encodes a polypeptide, a peptide fragment, or a coupled protein of the invention may lead to a humoral and cell-mediated immune response to the encoded antigen. It is thought that some bone marrow-derived professional antigen presenting cells are transfected by the nucleic acid construct and the encoded antigen is transcribed and translated into an immunogenic polypeptide that elicits specific responses. A feature of nucleic acid vaccines is that they provide for eliciting strong cytotoxic T-lymphocyte (CTL) responses. These responses occur because the nucleic acid-encoded polypeptides are synthesized in the cytosol of transfected cells. Furthermore, nucleic acid constructs that are produced in bacteria are rich in unmethylated CpG nucleotides that are recognized as foreign by macrophages. Thus, they elicit an innate immune response that enhances adaptive immunity. Therefore, nucleic acid vaccines are effective even when administered without adjuvants.

Direct injection of an expression cassette into living host cells transforms a number of the cells and causes them to express the introduced nucleic acid and thereby express a gene product. The transfected cells may display fragments of the expressed antigens on their cell surfaces together with major histocompatibility class I (MHC I) or class II (MHC II) complexes.

Nucleic acid constructs can be introduced into cells more efficiently by inducing muscle degeneration prior to the injection of the nucleic acid construct into an animal, including a human (Vitadello et. al., Hum. Gene. Ther., 5:11 (1994); Danko and Wolff, Vaccine, 12:1499 (1994); Davis et. al., Hum. Gene. Ther., 4:733 (1993)). For example, such a treatment is thought to increase the efficiency of transfer by up to 40 fold. Two of the most commonly used myonecrotic agents are the local anesthetic bupivicaine, and cardiotoxin (Danko and Wolff, Vaccine, 12:1499 (1994); Davis et. al., Hum. Gene. Ther., 4:733 (1993)). A number of other techniques have been employed to transfer nucleic acid constructs to muscle. Such other techniques include retroviral vectors, adenoviral vectors, and liposomes. However, direct injection of naked nucleic acid appears to be the most efficient of these delivery mechanisms at transferring and expressing foreign nucleic acids in cells.

Nucleic acid constructs can be administered in a pharmaceutically acceptable carrier. Pharmaceutically acceptable carriers are biologically compatible vehicles which are suitable for administration to a human or other mammalian subject, e.g., physiological saline. A therapeutically effective amount is an amount of the nucleic acid construct that is capable of producing an immune response (e.g., an enhanced T-cell response or antibody production) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of a nucleic acid construct is from approximately 106 to 1012 copies of the nucleic acid construct. This does can be repeatedly administered, as needed.

Numerous routes of administration may be used to administer nucleic acid constructs. Examples of such routes include intramuscular injection, intravenous, intraperitoneal, intradermal, intranasal and subcutaneous injection of nucleic acid constructs have all resulted in immunization against influenza virus hemagglutinin (HA) in chickens (reviewed in Pardoll and Beckerkleg, Immunity 3 (1995), 165-169). Nucleic acid based vaccines can also be administered through use of a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The nucleic acid construct is encapsulated in these microparticles, which are taken up by macrophages and gradually biodegraded within the cell, thereby releasing the nucleic acid construct. Once released, the nucleic acid is expressed within the cell. Another way to achieve uptake of a nucleic acid construct is through use of liposomes. Such liposomes can be prepared by standard methods. The nucleic acid constructs can be incorporated alone into these delivery vehicles or co-incorporated with tissue-specific antibodies. Alternatively, a molecular conjugate can be prepared that is composed of a nucleic acid construct attached to poly-L-lysine by electrostatic or covalent forces. Poly-L-lysine binds to a ligand that can bind to a receptor on target cells. Cristiano et al. (1995), J. Mol. Med. 73, 479). Alternatively, lymphoid tissue specific targeting can be achieved by the use of lymphoid tissue-specific transcriptional regulatory elements (TRE) such as a B lymphocyte, T lymphocyte, or dendritic cell specific TRE. Lymphoid tissue specific TRE are known (Thompson et al., Mol. Cell. Biol., 12:1043 (1992); Todd et al., J. Exp. Med., 177:1663 (1993); Penix et al., J. Exp. Med., 178:1483 (1993)).

The invention also provides microbe based vaccines. Generally, these vaccines relate to microbes that have been transformed with a nucleic acid construct that provides for the expression of a polypeptide, a peptide fragment, or a coupled protein of the invention. For example, Listeria monocytogenes may be used as a vector to elicit T-cell immunity. This is because it infects antigen-presenting cells and also because infection originates at the mucosa. Lieberman and Frankel, Vaccine, 20:2007-10 (2002). According, Listeria may be transformed with a nucleic acid construct that provides for the expression of a polypeptide, a peptide fragment, or a coupled protein that elicits an immune response against the spike protein from the coronavirus that causes severe acute respiratory syndrome. Highly attenuated forms of Listeria may be constructed according to methods reported in the art. Lieberman and Frankel, Vaccine, 20:2007 (2002). Salmonella may also be used as a vector to elicit a cytotoxic T lymphocyte (CTL) response against the coronavirus that causes severe acute respiratory syndrome. Pasetti et al., Infect Immun., 70:4009 (2002).

An immune composition or vaccine may be administered by any conventional route used in the field of vaccines. For example, an immune composition or vaccine can be administered orally or by intravenous infusion, or injected subcutaneously, intramuscularly, intraperitoneally, intrarectally, intravaginally, intranasally, intragastrically, intratracheally, or intrapulmonarily. The choice of the administration route depends on a number of parameters such as the nature of the active principle; the identity of the polypeptide, peptide fragment, peptidomimetic, coupled protein, immunopeptide, DNA vaccine; or the adjuvant that is combined with the aforementioned molecules. Administration of an immune composition may take place in a single dose or in a dose repeated once or several times over a certain period. The appropriate dosage varies according to various parameters. Such parameters include the individual treated (adult or child), the immune composition or antigen itself, the mode and frequency of administration, the presence or absence of adjuvant and, if present, the type of adjuvant and the desired effect (e.g. protection or treatment), as will be determined by persons skilled in the art.

IV. Antibodies and Aptamers of the Invention

The invention provides antibodies that bind to an amino acid sequence as set forth in SEQ ID NO: 1, 13, 14, 15, 20-59, 60, 61, 62, 63, 66, 69 or a fragment of SEQ ID NO: 1, or conservative variants thereof. Such antibodies are useful for the diagnosis, immunization against, and treatment of severe acute respiratory syndrome (SARS). In some embodiments, the antibody binds to a peptide having SEQ ID NO:58 or 59. Antibodies that bind to the P540 peptide (SEQ ID NO: 59) are highly effective, and can detect spike polypeptides even after extensive dilution. For example, a P540 antibody preparation diluted 1:10,000 could still detect spike polypeptides.

Antibodies can be prepared using an intact polypeptide or peptide fragment of interest as the immunizing antigen. The polypeptide or fragment used to immunize an animal can be derived from translated cDNA or chemical synthesis. A polypeptide or peptide fragment can be coupled to a carrier protein, if desired. Such commonly used carrier proteins which are chemically coupled to the peptide include keyhole limpet hemocyanin (KLH), thyroglobulin, bovine serum albumin (BSA), and tetanus toxoid. A coupled protein can be used to immunize the animal (e.g., a mouse, a rat, or a rabbit).

If desired, polyclonal or monoclonal antibodies can be further purified, for example, by binding to and elution from a matrix to which the polypeptide or peptide fragment to which the antibodies were raised is bound. Those of skill in the art will know of various techniques common in the immunology arts for purification and/or concentration of polyclonal antibodies, as well as monoclonal antibodies (Coligan, et al., Unit 9, Current Protocols in Immunology, Wiley Interscience, 1991, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

An antibody suitable for binding to a polypeptide or peptide fragment is specific for at least one portion of a region of the polypeptide. For example, one of skill in the art can use a peptide fragment to generate appropriate antibodies of the invention. Antibodies of the invention include polyclonal antibodies, monoclonal antibodies, and fragments of polyclonal and monoclonal antibodies.

The preparation of polyclonal antibodies is well-known to those skilled in the art (Green et al., Production of Polyclonal Antisera, in Immunochemical Protocols (Manson, ed.), pages 1-5 (Humana Press 1992); Coligan et al., Production of Polyclonal Antisera in Rabbits, Rats, Mice and Hamsters, in Current Protocols in Immunology, section 2.4.1 (1992), which are hereby incorporated by reference). For example, a polypeptide or peptide fragment is injected into an animal host, preferably according to a predetermined schedule incorporating one or more booster immunizations, and the animal is bled periodically. Polyclonal antibodies specific for the polypeptide or peptide fragment may then be purified from such antisera by, for example, affinity chromatography using the polypeptide or peptide fragment coupled to a suitable solid support.

The preparation of monoclonal antibodies likewise is conventional (Kohler & Milstein, Nature, 256:495 (1975); Coligan et al., sections 2.5.1-2.6.7; and Harlow et al., Antibodies: A Laboratory Manual, page 726 (Cold Spring Harbor Pub. 1988)), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (Coligan et al., sections 2.7.1-2.7.12 and sections 2.9.1-2.9.3; Barnes et al., Purification of Immunoglobulin G (IgG), in Methods in Molecular Biology, Vol. 10, pages 79-104 (Humana Press 1992)). Methods of in vitro and in vivo multiplication of monoclonal antibodies is well-known to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an air reactor, in a continuous stirrer reactor, or immobilized or entrapped cell culture. Multiplication in vivo may be carried out by injecting cell clones into mammals histocompatible with the parent cells, e.g., osyngeneic mice, to cause growth of antibody-producing tumors. Optionally, the animals are primed with a hydrocarbon, especially oils such as pristine tetramethylpentadecane prior to injection. After one to three weeks, the desired monoclonal antibody is recovered from the body fluid of the animal.

Antibodies can also be prepared through use of phage display techniques. In one example, an organism is immunized with an antigen, such as a polypeptide or peptide fragment of the invention. Lymphocytes are isolated from the spleen of the immunized organism. Total. RNA is isolated from the splenocytes and mRNA contained within the total RNA is reverse transcribed into complementary deoxyribonucleic acid (cDNA). The cDNA encoding the variable regions of the light and heavy chains of the immunoglobulin is amplified by polymerase chain reaction (PCR). To generate a single chain fragment variable (scFV) antibody, the light and heavy chain amplification products may be linked by splice overlap extension PCR to generate a complete sequence and ligated into a suitable vector. E. coli are then transformed with the vector encoding the scFV, and are infected with helper phage, to produce phage particles that display the antibody on their surface. Alternatively, to generate a complete antigen binding fragment (Fab), the heavy chain amplification product can be fused with a nucleic acid sequence encoding a phage coat protein, and the light chain amplification product can be cloned into a suitable vector. E. coli expressing the heavy chain fused to a phage coat protein are transformed with the vector encoding the light chain amplification product. The disulphide linkage between the light and heavy chains are established in the periplasm of E. coli. The result of this procedure is to produce an antibody library with up to 109 clones. The size of the library can be increased to 1018 phage by later addition of the immune responses of additional immunized organisms that may be from the same or different hosts. Antibodies that recognize a specific antigen can be selected through panning. Briefly, an entire antibody library can be exposed to an immobilized antigen against which antibodies are desired. Phage that do not express an antibody that binds to the antigen are washed away. Phage that express the desired antibodies are immobilized on the antigen. These phage are then eluted and again amplified in E. coli. This process can be repeated to enrich the population of phage that express antibodies that specifically bind to the antigen. After phage are isolated that express an antibody that binds to an antigen, a vector containing the coding sequences for the antibody can be isolated from the phage particles and the coding sequences can be recloned into a suitable vector to produce an antibody in soluble form. In another example, a human phage library can be used to select for antibodies, such as monoclonal antibodies, that bind to the spike protein from SARS-CoV. Briefly, splenocytes may be isolated from a human that is infected, or not infected, with SARS-CoV and used to create a human phage library according to methods as described above and known in the art. These methods may be used to obtain human monoclonal antibodies that bind to the spike protein of SARS-CoV. Phage display methods to isolate antigens and antibodies are known in the art and have been described (Gram et al., Proc. Natl. Acad. Sci., 89:3576 (1992); Kay et al., Phage display of peptides and proteins: A laboratory manual. San Diego: Academic Press (1996); Kermani et al., Hybrid, 14:323 (1995); Schmitz et al., Placenta, 21 Suppl. A:S106 (2000); Sanna et al., Proc. Natl. Acad. Sci., 92:6439 (1995)).

An antibody of the invention may be derived from a “humanized” monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described (Orlandi et al., Proc. Nat'l Acad. Sci. USA, 86:3833 (1989) which is hereby incorporated in its entirety by reference). Techniques for producing humanized monoclonal antibodies are described (Jones et al., Nature, 321:522 (1986); Riechmann et al., Nature, 332:323 (1988); Verhoeyen et al, Science, 239:1534 (1988); Carter et al., Proc. Nat'l Acad. Sci. USA, 89:4285 (1992); Sandhu, Crit. Rev. Biotech., 12:437 (1992); and Singer et al., J. Immunol., 150:2844 (1993), which are hereby incorporated by reference).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described (Green et al., Nature Genet., 7:13 (1994); Lonberg et al., Nature, 368:856 (1994); and Taylor et al., Int. Immunol., 6:579 (1994), which are hereby incorporated by reference).

Antibody fragments of the invention can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described (U.S. Pat. Nos. 4,036,945; 4,331,647; and 6,342,221, and references contained therein; Porter, Biochem. J., 73:119 (1959); Edelman et al., Methods in Enzymology, Vol. 1, page 422 (Academic Press 1967); and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise, an association of V_{H} and V_{L} chains. This association may be noncovalent (Inbar et al., Proc. Nat'l Acad. Sci. USA, 69:2659 (1972)). Alternatively, the variable chains can be linked by an intermolecular disulfide bond or cross-linked by chemicals such as glutaraldehyde (Sandhu, Crit. Rev. Biotech., 12:437 (1992)). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described (Whitlow et al., Methods: A Companion to Methods in Enzymology, Vol. 2, page 97 (1991); Bird et al., Science, 242:423 (1988), Ladner et al., U.S. Pat. No. 4,946,778; Packet al., Bio/Technology, 11:1271(1993); and Sandhu, Crit. Rev. Biotech., 12:437 (1992)).

Another form of an antibody fragment is a peptide that forms a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: A Companion to Methods in Enzymology, Vol. 2, page 106 (1991)).

An antibody of the invention may be coupled to a toxin. Such antibodies may be used to treat animals, including humans, that are infected with the virus that is etiologically linked to severe acute respiratory syndrome. For example, an antibody that binds to the spike protein of the coronavirus that is etiologically linked to severe acute respiratory syndrome may be coupled to a tetanus toxin and administered to an animal suffering from infection by the aforementioned virus. The toxin-coupled antibody is thought to bind to a portion of a spike protein presented on an infected cell, and then kill the infected cell.

An antibody of the invention may be coupled to a detectable tag. Such antibodies may be used within diagnostic assays to determine if an animal, such as a human, is infected with SARS-CoV. Examples of detectable tags include, fluorescent proteins (i.e., green fluorescent protein, red fluorescent protein, yellow fluorescent protein), fluorescent markers (i.e., fluorescein isothiocyanate, rhodamine, texas red), radiolabels (i.e., ³H, ³²P, ¹²⁵I), enzymes (i.e., β-galactosidase, horseradish peroxidase, β-glucuronidase, alkaline phosphatase), or an affinity tag (i.e., avidin, biotin, streptavidin). Methods to couple antibodies to a detectable tag are known in the art. Harlow et al., Antibodies: A Laboratory Manual, page 319 (Cold Spring Harbor Pub. 1988).

The invention also provides aptamers to the polypeptides and peptide fragments of the invention. Aptamers of the invention can be peptide or nucleic acid aptamers. Peptide aptamers are peptides that bind to a polypeptide or peptide fragment of the invention with affinities that are often comparable to those for monoclonal antibody-antigen complexes. Similarly, nucleic acid aptamers are nucleic acids that bind to a polypeptide or peptide fragment of the invention with strong affinities, for example, affinities that are often comparable to those for monoclonal antibody-antigen complexes.

In one example, nucleic acid aptamers can be isolated through use of a library of random oligonucleotide sequences. The library is screened to ascertain which oligonucleotide binds to the S polypeptides and peptide fragments of the invention. The bound oligonucleotides are eluted from the immobilized polypeptides or peptide fragments and are then amplified by PCR. This process may be repeated to select for aptamers having high affinity for the polypeptides and peptide fragments of the invention. The sequence of the nucleic acid coding for the aptamers can then be determined and cloned into a suitable vector to facilitate production and maintenance of the desired aptamer.

Peptide aptamers can be isolated by mRNA display of a library that contains a promoter, a start codon, a nucleic acid sequence that encodes random peptides. In some embodiments, the DNA library also includes a nucleic acid segment that codes for a histidine tag. This library is transcribed using a suitable polymerase, such as T7 RNA polymerase, after which a puromycin-containing poly A linker is ligated onto the 3′ end of the newly formed mRNAs. When these mRNAs are translated in vitro, the nascent peptides form covalent bonds to the puromycin of the linker to form an mRNA-peptide fusion molecule. The mRNA-peptide fusion molecules are then purified through use of Ni-NTA agarose and oligo-dT-cellulose. The mRNA portion of the fusion molecule is then reverse transcribed. The double-stranded DNA/RNA-peptide fusion molecules are then incubated with a polypeptide or peptide fragment of the invention and unbound fusion molecules are washed away. The bound fusion molecules are eluted from the immobilized polypeptides or peptide fragments and are then amplified by PCR. This process may be repeated to select for aptamers having high affinity for the polypeptides and peptide fragments of the invention. The sequence of the nucleic acid coding for the aptamers can then be determined and cloned into a suitable vector. Methods for the preparation of peptide aptamers have been described (Wilson et al., Proc. Natl. Acad. Sci., 98:3750 (2001)). Accordingly, the invention provides aptamers that recognize the polypeptides and peptide fragments of the invention.

V. Pharmaceutical Compositions of the Invention

The invention provides pharmaceutical compositions containing an antibody that binds to an amino acid sequence as set forth in SEQ ID NO: 1, 13, 14, 15, 20-59, 60, 61, 62, 63, 66, 69 or a fragment of SEQ ID NO: 1, or a conservative variant thereof, and a pharmaceutically acceptable carrier. In some embodiments, the antibody binds to a peptide having SEQ ID NO:58 or 59. Antibodies that bind to the P540 peptide (SEQ ID NO:59) are highly effective, and can detect spike polypeptides even after extensive dilution. For example, a P540 antibody preparation at dilution 1:10,000 could still detect spike polypeptides.

The pharmaceutical compositions of the invention may be prepared in many forms that include tablets, hard or soft gelatin capsules, aqueous solutions, suspensions, and liposomes and other slow-release formulations, such as shaped polymeric gels. An oral dosage form may be formulated such that the antibody is released into the intestine after passing through the stomach. Such formulations are described in U.S. Pat. No. 6,306,434 and in the references contained therein.

Oral liquid pharmaceutical compositions may be in the form of, for example, aqueous or oily suspensions, solutions, emulsions, syrups or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives.

An antibody can be formulated for parenteral administration (e.g., by injection, for example, bolus injection or continuous infusion) and may be presented in unit dosage form in ampules, prefilled syringes, small volume infusion containers or multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Pharmaceutical compositions suitable for rectal administration can be prepared as unit dose suppositories. Suitable carriers include saline solution and other materials commonly used in the art.

For administration by inhalation, an antibody can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, an antibody may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, an antibody may be administered via a liquid spray, such as via a plastic bottle atomizer.

Pharmaceutical compositions of the invention may also contain other ingredients such as flavorings, colorings, anti-microbial agents, or preservatives. It will be appreciated that the amount of an antibody required for use in treatment will vary not only with the particular carrier selected but also with the route of administration, the nature of the condition being treated and the age and condition of the patient. Ultimately the attendant health care provider may determine proper dosage. In addition, a pharmaceutical composition may be formulated as a single unit dosage form.

VI. Method to Immunize, Treat, and Diagnose an Animal Against Severe Acute Respiratory Syndrome

The invention provides a method to immunize an animal against severe acute respiratory syndrome. The method relates to administering a therapeutically effective amount of an antibody that binds to an amino acid sequence as set forth in SEQ ID NO: 1, 13, 14, 15, 20-59, 60, 61, 62, 63, 66, 69 or a fragment of SEQ ID NO: 1, or a conservative variant thereof to an animal; administering an effective amount of an immune composition to an animal; administering an effective amount of a viral vaccine to an animal; or administering an effective amount of a nucleic acid vaccine to an animal. The animal may be a mammal, such as a human. Methods to administer vaccines and immune compositions have been described herein and are known in the art.

An animal may also be treated for infection by SARS-CoV through passive immunization according to the invention. For example, antibodies that bind to an amino acid sequence as set forth in SEQ ID NO: 1, 13, 14, 15, 20-55, 60, 61, 62, 63, 66, 69 or a fragment of SEQ ID NO: 1, or a conservative variant thereof may be administered to an animal, such as a human, that is infected with SARS-CoV. Such administration may be suitable in situations where a patient is immune compromised and is unable to mount an effective immune response against SARS-CoV, or to a vaccine or immune composition.

The invention provides a method to diagnose severe acute respiratory syndrome in an animal that involves contacting a biological sample obtained from the animal, such as tissue samples, blood, mucus, or saliva, with an antibody that binds to an amino acid sequence as set forth in SEQ ID NO: 1, 13, 14, 15, 20-59, 60, 61, 62, 33 or a fragment of SEQ ID NO: 1, and determining if the antibody binds to the biological sample. Diagnostic assays that utilize antibodies to detect the presence of an antigen in a biological sample are well known in the art. Briefly, an antibody of the invention may be immobilized on a surface. A biological sample can then be contacted with the immobilized antibody such that an antigen contained in the sample is bound by the antibody to form an antibody-antigen complex. The sample may then be optionally washed to remove unbound materials. A second antibody of the invention that is coupled to a detectable tag, such as an enzyme or radiolabel, can then be contacted with the antibody-antigen complex such that the enzyme or radiolabel is immobilized on the surface. The detectable tag can then be detected to determine if an antigen was present in the biological sample. In another example, a biological sample can be immobilized on a surface. An antibody of the invention that is coupled to a detectable tag is then contacted with the immobilized biological sample and any unbound material is washed away. The presence of the detectable tag is then detected to determine whether the biological sample contained an antigen. Examples of such assays are well known in the art and include, enzyme-linked immunosorbant assays, radioimmuno assays, and the like.

Nucleic acid based methods may also be used to diagnose severe acute respiratory syndrome. In one example, polymerase chain reaction (PCR) may be used to diagnose SARS-CoV infection. Briefly, a biological sample, such as a tissue sample, blood, mucus, or saliva, is obtained from an animal. The nucleic acids within the sample are then extracted using common methods, such as organic extraction. The extracted nucleic acids are then mixed with forward and reverse primers that anneal to nucleic acids that encode SARS proteins, polymerase, nucleotides, and typically a buffer that includes components that allow the polymerase to extend the forward and reverse primers using the SARS nucleic acid as a template. The presence of amplified DNA between the forward and reverse primers is then detected to determine if the sample contained SARS originated nucleic acid. Nucleic acid hybridization techniques, such as Northern and Southern blotting, may also be used to detect the presence of SARS nucleic acids in a biological sample.

VII. Kits

The invention provides a kit which contains packaging material and an antibody that binds to an amino acid sequence as set forth in SEQ ID NO: 1, 13, 14, 15, 45, 46, or 47, 58, 59, 61, 62, 63, 66, 69 or a fragment of SEQ ID NO: 1, or a conservative variant thereof. The kit may also contain a syringe to allow for injection of the antibody contained within the kit into an animal, such as a human. In another embodiment, the invention provides a kit that may contain packaging material, and an antibody that binds to an amino acid sequence as set forth in SEQ ID NO: 1, 13, 14, 15, 20-59, 60, 61, 62, 63, 66, 69 or a fragment of SEQ ID NO: 1, or a conservative variant thereof that is formulated for administration to an animal, such as a human. In some embodiments, the antibody binds to an amino acid sequence set forth in SEQ ID NO:59. In other embodiments, the antibody binds to an amino acid sequence as set forth in SEQ ID NO:58. Such a kit may optionally contain a syringe to allow for injection of the antibody contained within the kit into an animal, such as a human.

The invention also provides a kit which contains packaging material and DNA vaccine having a DNA molecule or expression vector encoding a polypeptide with an amino acid sequence as set forth in SEQ ID NO: 1, 13, 14, 15, 45, 46, or 47, 58, 59, 61, 62, 63, 66, 69 or a fragment of SEQ ID NO: 1, or a conservative variant thereof. The kit may also contain a device for administering the DNA vaccine (e.g. a syringe or gene gun) to allow for administration of the vaccine contained within the kit into an animal, such as a human.

The invention also provides a kit which contains packaging material and vaccine composition that includes a polypeptide with an amino acid sequence as set forth in SEQ ID NO: 1, 13, 14, 15, 45, 46, or 47, 58, 59, 61, 62, 63, 66, 69 or a fragment of SEQ ID NO: 1, or a conservative variant thereof. The kit may also contain a device for administering the vaccine (e.g. a syringe) to allow for administration of the vaccine contained within the kit into an animal, such as a human.

The invention also provides a kit for detecting SARS-CoV infection, which contains packaging material and a polypeptide with an amino acid sequence as set forth in SEQ ID NO: 1, 13, 14, 15, 45, 46, or 47, 58, 59, 61, 62, 63, 66, 69 or a fragment of SEQ ID NO: 1, or a conservative variant thereof. The polypeptide(s) can be immobilized onto a solid support. Such a kit may be used for detection of antibodies directed against the SARS-CoV in the serum of infected animals or humans. The kit can also contain a means for detecting binding of such antibodies to the S polypeptide(s).

VIII. Amino Acid Sequence of a Full-Length Spike (S) Protein (Amino Acids 1-1255) from the Tor2 Isolate of the SARS-CoV Virus

MFIFLLFLTLTSGSDLDRCTTFDDVQAPNYTQHTSSMRGVYYPDEIFRSD
TLYLTQDLFLPFYSNVTGFHTINHTFGNPVIPFKDGIYFAATEKSNVVRG
WVFGSTMNNKSQSVIIINNSTNVVIRACNFELCDNPFFAVSKPMGTQTHT
MIFDNAFNCTFEYISDAFSLDVSEKSGNFKHLREFVFKNKDGFLYVYKGY
QPIDVVRDLPSGFNTLKPIFKLPLGINITNERAILTAFSPAQDIWGTSAA
AYFVGYLKPTTFMLKYDENGTITDAVDCSQNPLAELKCSVKSFEIDKGIY
QTSNFRVVPSGDVVRFPNITNLCPFGEVFNATKFPSVYAWERKKISNCVA
DYSVLYNSTFFSTFKCYGVSATKLNDLCFSNVYADSFVVKGDDVRQIAPG
QTGVIADYNYKLPDDFMGCVLAWNTRNIDATSTGNYNYKYRYLRHGKLRP
FERDISNVPFSPDGKPCTPPALNCYWPLNDYGFYTTTGIGYQPYRVVVLS
FELLNAPATVCGPKLSTDLIKNQCVNFNFNGLTGTGVLTPSSKRFQPFQQ
FGRDVSDFTDSVRDPKTSEILDISPCAFGGVSVITPGTNASSEVAVLYQD
VNCTDVSTAIHADQLTPAWRIYSTGNNVFQTQAGCLIGAEHVDTSYECDI
PIGAGICASYHTVSLLRSTSQKSIVAYTMSLGADSSIAYSNNTIAIPTNF
SISITTEVMPVSMAKTSVDCNMYICGDSTECANLLLQYGSFCTQLNRALS
GIAAEQDRNTREVFAQVKQMYKTPTLKYFGGENFSQILPDPLKPTKRSFI
EDLLFNKVTLADAGFMKQYGECLGDINARDLICAQKFNGLTVLPPLLTDD
MIIAAYTAALVSGTATAGWTFGAGAALQIPFAMQMAYRFNGIGVTQNVLY
ENQKQIANQFNKAISQIQESLTTTSTALGKLQDVVNQNAQALNTLVKQLS
SNFGAISSVLNDILSRLDKVEAEVQIDRLITGRIQSLQTYVTQQLIRAAE
IRASANLAATKMSECVLGQSKRVDFCGKGYHLMSFPQAAPHGVVFLHVTY
VPSQERNFTTAPAICHEGKAYFPREGVFVFNGTSWFITQRNFFSPQIITT
DNTFVSGNCDVVIGIINNTVYDPLQPELDSFKEELDKYFKNHTSPDVDLG
DISGINASVVNIQKEIDRLNEVAKNLNESLIDLQELGKYEQYIKWPWYVW
LGFIAGLIAIVMVTILLCCMTSCCSCLKGACSCGSCCKFDEDDSEPVLKG
VKLHYT

IX. Nucleic Acid Sequence of a Full-Length Spike (S) Protein (Nucleotides 1-3768)

(SEQ ID NO:2)

ATGTTTATTTTCTTATTATTTCTTACTCTCACTAGTGGTAGTGACCTTGA
CCGGTGCACCACTTTTGATGATGTTCAAGCTCCTAATTACACTCAACATA
CTTCATCTATGAGGGGGGTTTACTATCCTGATGAAATTTTTAGATCAGAC
ACTCTTTATTTAACTCAGGATTTATTTCTTCCATTTTATTCTAATGTTAC
AGGGTTTCATACTATTAATCATACGTTTGGCAACCCTGTCATACCTTTT
AAGGATGGTATTTATTTTGCTGCCACAGAGAAATCAAATGTTGTCCGTGG
TTGGGTTTTTGGTTCTACCATGAACAACAAGTCACAGTCGGTGATTATTA
TTAACAATTCTACTAATGTTGTTTATACGAGCATGTAACTTTGAATTGTG
TGACAACCCTTTCTTTGCTGTTTCTAAACCCATGGGTACACAGACACATA
CTATGATATTCGATAATGCATTTAATTGCACTTTCGAGTACATATCTGAT
GCCTTTTCGCTTGATGTTTCAGAAAAGTCAGGTAATTTTAAACACTTACG
AGAGTTTGTGTTTAAAAATAAAGATGGGTTTCTCTATGTTTATAAGGGCT
ATCAACCTATAGATGTAGTTCGTGATCTACCTTCTGGTTTTAACACTTTG
AAACCTATTTTTAAGTTGCCTCTTGGTATTAACATTACAAATTTTAGAGC
CATTCTTACAGCCTTTTCACCTGCTCAAGACATTTGGGGCACGTCAGCTG
CAGCCTATTTTGTTGGCTATTTAAAGCCAACTACATTTATGCTCAAGTAT
GATGAAAATGGTACAATCACAGATGCTGTTGATTGTTCTCAAAATCCACT
TGCTGAACTCAAATGCTCTGTTAAGAGCTTTGAGATTGACAAAGGAATTT
ACCAGACCTCTAATTTCAGGGTTGTTCCCTCAGGAGATGTTGTGAGATTC
CCTAATATTACAAACTTGTGTCCTTTTGGAGAGGTTTTTAATGCTACTAA
ATTCCCTTCTGTCTATGCATGGGAGAGAAAAAAAATTTCTAATTGTGTTG
CTGATTACTCTGTGCTCTACAACTCAACATTTTTTTCAACCTTTAAGTGC
TATGGCGTTTCTGCCACTAAGTTGAATGATCTTTGCTTCTCCAATGTCTA
TGCAGATTCTTTTGTAGTCAAGGGAGATGATGTAAGACAAATAGCGCCAG
GACAAACTGGTGTTATTGCTGATTATAATTATAAATTGCCAGATGATTTC
ATGGGTTGTGTCCTTGCTTGGAATACTAGGAACATTGATGCTACTTCAAC
TGGTAATTATAATTATAAATATAGGTATCTTAGACATGGCAAGCTTAGGC
CCTTTGAGAGAGACATATCTAATGTGCCTTTCTCCCCTGATGGCAAACCT
TGCACCCCACCTGCTCTTAATTGTTATTGGCCATTAAATGATTATGGTTT
TTACACCACTACTGGCATTGGCTACCAACCTTACAGAGTTGTAGTACTTT
CTTTTGAACTTTTAAATGCACCGGCCACGGTTTGTGGACCAAAATTATCC
ACTGACCTTATTAAGAACCAGTGTGTCAATTTTAATTTTAATGGACTCAC
TGGTACTGGTGTGTTAACTCCTTCTTCAAAGAGATTTCAACCATTTCAAC
AATTTGGCCGTGATGTTTCTGATTTCACTGATTCCGTTCGAGATCCTAAA
ACATCTGAAATATTAGACATTTCACCTTGCGCTTTTGGGGGTGTAAGTGT
AATTACACCTGGAACAAATGCTTCATCTGAAGTTGCTGTTCTATATCAAG
ATGTTAACTGCACTGATGTTTCTACAGCAATTCATGCAGATCAACTCACA
CCAGCTTGGCGCATATATTCTACTGGAAACAATGTATTCCAGACTCAAGC
AGGCTGTCTTATAGGAGCTGAGCATGTCGACACTTCTTATGAGTGCGACA
TTCCTATTGGAGCTGGCATTTGTGCTAGTTACCATACAGTTTCTTTATTA
CGTAGTACTAGCCAAAAATCTATTGTGGCTTATACTATGTCTTTAGGTGC
TGATAGTTCAATTGCTTACTCTAATAACACCATTGCTATACCTACTAACT
TTTCAATTAGCATTACTACAGAAGTAATGCCTGTTTCTATGGCTAAAACC
TCCGTAGATTGTAATATGTACATCTGCGGAGATTCTACTGAATGTGCTAA
TTTGCTTCTCCAATATGGTAGCTTTTGCACACAACTAAATCGTGCACTCT
CAGGTATTGCTGCTGAACAGGATCGCAACACACGTGAAGTGTTCGCTCAA
GTCAAACAAATGTACAAAACCCCAACTTTGAAATATTTTGGTGGTTTTAA
TTTTTCACAAATATTACCTGACCCTCTAAAGCCAACTAAGAGGTCTTTTA
TTGAGGACTTGCTCTTTAATAAGGTGACACTCGCTGATGCTGGCTTCATG
AAGCAATATGGCGAATGCCTAGGTGATATTAATGCTAGAGATCTCATTTG
TGCGCAGAAGTTCAATGGACTTACAGTGTTGCCACCTCTGCTCACTGATG
ATATGATTGCTGCCTACACTGCTGCTCTAGTTAGTGGTACTGCCACTGCT
GGATGGACATTTGGTGCTGGCGCTGCTCTTCAAATACCTTTTGCTATGCA
AATGGCATATAGGTTCAATGGCATTGGAGTTACCCAAAATGTTCTCTATG
AGAACCAAAAACAAATCGCCAACCAATTTAACAAGGCGATTAGTCAAATT
CAAGAATCACTTACAACAACATCAACTGCATTGGGCAAGCTGCAAGACGT
TGTTAACCAGAATGCTCAAGCATTAAACACACTTGTTAAACAACTTAGCT
CTAATTTTGGTGCAATTTCAAGTGTGCTAAATGATATCCTTTCGCGACTT
GATAAAGTCGAGGCGGAGGTACAAATTGACAGGTTAATTACAGGCAGACT
TCAAAGCCTTCAAACCTATGTAACACAACAACTAATCAGGGCTGCTGAAA
TCAGGGCTTCTGCTAATCTTGCTGCTACTAAAATGTCTGAGTGTGTTCTT
GGACAATCAAAAAGAGTTGACTTTTGTGGAAAGGGCTACCACCTTATGTC
CTTCCCACAAGCAGCCCCGCATGGTGTTGTCTTCCTACATGTCACGTATG
TGCCATCCCAGGAGAGGAACTTCACCACAGCGCCAGCAATTTGTCATGAA
GGCAAAGCATACTTCCCTCGTGAAGGTGTTTTTGTGTTTAATGGCACTTC
TTGGTTTATTACACAGAGGAACTTCTTTTCTCCACAAATAATTACTACAG
ACAATACATTTGTCTCAGGAAATTGTGATGTCGTTATTGGCATCATTAAC
AACACAGTTTATGATCCTCTGCAACCTGAGACTCGACTCATTCAAAGAAG
AGCTGGACAAGTACTTCAAAAATCATACATCACCAGATGTTGATCTTGGC
GACATTTCAGGCATTAACGCTTCTGTCGTCAACATTCAAAAAGAAATTGA
CCGCCTCAATGAGGTCGCTAAAAATTTAAATGAATCACTCATTGACCTTC
AAGAATTGGGAAAATATGAGCAATATATTAAATGGCCTTGGTATGTTTGG
CTCGGCTTCATTGCTGGACTAATTGCCATCGTCATGGTTACAATCTTGCT
TTGTTGCATGACTAGTTGTTGCAGTTGCCTCAAGGTGCATGCTCTTGTGT
TCTTGCTGCAAGTTTGATGAGGATGACTCTGAGCCAGTTCTCAAGGGTGT
CAAATTACATTACACATAA

### EXAMPLE 1

### Cloning of the Spike Protein

The nucleic acid sequence encoding the full length spike protein was obtained through use of overlapping polymerase chain reaction (PCR). Overlapping clones containing fragments of the spike protein were obtained from the British Columbia Cancer Agency (Vancouver, British Columbia). The following primers were used during the PCR reactions to amplify the nucleic acid sequence encoding the full-length spike protein of SARS-CoV: Clone 1: Forward primer: 5′-A GTC GGA TCC GGT AGG CTT ATC ATT AGA G-3′ (SEQ ID NO: 3); Reverse primer: 5′-CCA TCA GGG GAG AAA GGC AC-3 (SEQ ID NO: 4). Clone 2: Forward primer: 5′-GTG CCT TTC TCC CCT GAT GG-3′ (SEQ ID NO: 5); Reverse primer: 5′-GAA GAG CAG CGC CAG CAC C-3′ (SEQ ID NO: 6). Clone 3: Forward primer: 5′-GGT GCT GGC GCT GCT CTT C-3′ (SEQ ID NO: 7); Reverse primer: 5′-A CTG TCT AGA GTT CGT TTA TGT GTA ATG-3 (SEQ ID NO: 8).

The nucleic acid segment that resulted from overlapping PCR between the nucleic acid segments generated with the above pairs of primers contain amino acid residues from number I to number 1255 of the spike protein of the virus (SARS-CoV) that is etiologically linked to severe acute respiratory syndrome. The underlined primer sequences represent restriction enzyme cutting sites for BamHI and XbaI that were used to clone the amplified fragment into pCDNA3(+) (Invitrogen, Carlsbad, Calif.).

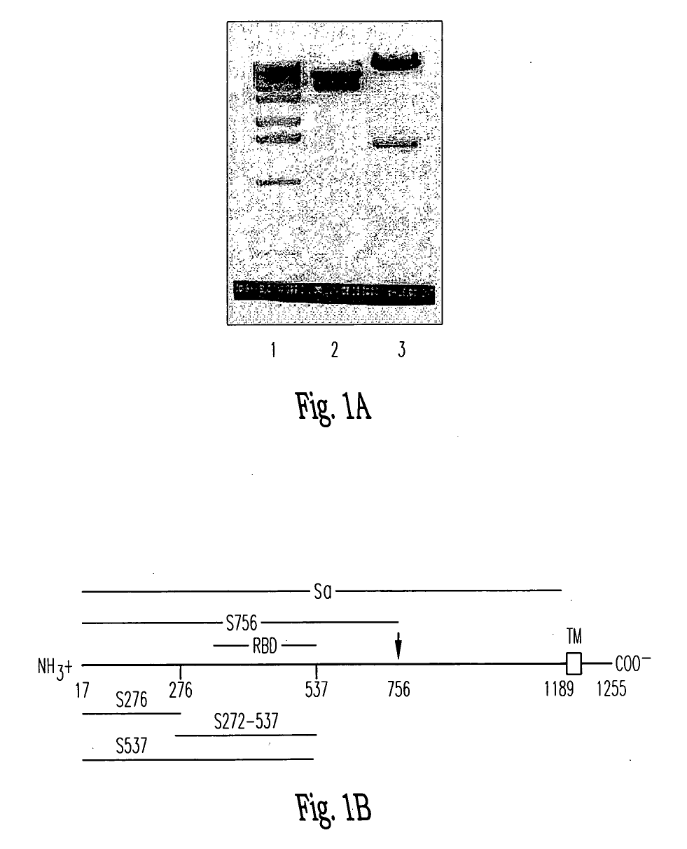

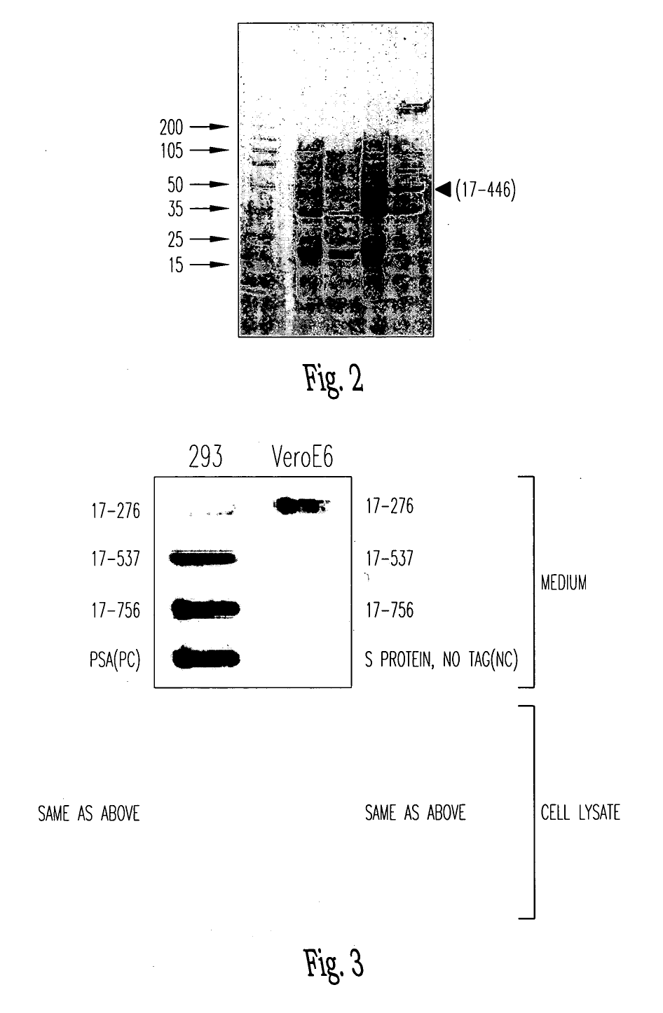

The full length spike protein gene has been cloned as shown in FIG. 1. FIG. 1 shows a gel for the nucleic acid segment encoding the full length spike protein inserted into the pCDNA3.1 (+) vector that has been digested with the restriction enzymes (Lane 2: BamHI and XbaI; Lane 3: HindIII).

### EXAMPLE 2

### Generation of Amino-Terminal (S1) and Carboxyl-Terminal (S2) Fragments of the Full Length Spike Protein

Computer analysis identified a potential functional separation site between the amino-terminus (S1) and the carboxyl-terminus (S2) of the spike protein. The separation site between S 1 and S2 is between positions between 758 and 761 (⁷⁵⁸RNTR⁷⁶¹) relative to SEQ ID NO: 1. PCR was used to create nucleic acids that code for the amino-terminal fragment (S1), and the carboxyl-terminal fragment (S2) of the spike protein.

The following primers, S1 forward primer 5′-AGTC GGA TCC GAC CGG TGC ACC ACT TTT G-3′ (SEQ ID NO: 9), and the reverse primer, S1 Reverse primer: 5′-AGTC GGG CCC CTG TTC AGC AGC AAT ACC-3′ (SEQ ID NO: 10), were used to prepare a nucleic acid segment coding for amino acid residues 17-757 of the spike protein. Two restriction sites, BamHI and ApaI, underlined in the two primers were used to clone the nucleic acid segment coding for the amino-terminal fragment of the spike protein (S1) gene into the pSecTag2B plasmid for expression.

The following pair of primers, S2 Forward: 5′-ACTG GGATCC GAA GTG TTC GCT CAA GTC-3′ (SEQ ID NO: 11), and S2 Reverse: 5′-ACTG TCTAGA TTG CTC ATA TTT TCC C-3′ (SEQ ID NO: 12), were used within a PCR reaction to prepare a nucleic acid segment coding for amino acid residues 762-1189 of the spike protein. Two restriction sites, BamHI and XbaI, underlined in the two primers were used to clone the nucleic acid segment coding for the carboxyl-terminal fragment of the spike protein (S2) gene into pCDNA3.1 (+) plasmid for expression.

To create a fragment containing residues 272-537, the following pair of primers was used for PCR amplification: primer 5′ GATCGGATCCGGTACAATCACAG 3′ (SEQ ID NO:64) and primer 5′ GATCGGGCCCGACACACTGGTTC 3′ (SEQ ID NO:65). The amplified fragment was digested with BamHI and ApaI and ligated into pSecTag2B digested with the same restriction enzymes. A schematic diagram of the position of many of the soluble spike protein fragments within the full-length spike protein is provided in FIG. 1B.

In some cases, nucleic acids encoding the S fragments and full-length S polypeptides had their native leader sequence (spike protein amino acids 1-16, MFIFLLFLTLTSGSDL (SEQ ID NO:60)) replaced with a mouse k chain leader sequence (METDTLLLWVLLLWVPGSTGD) (SEQ ID NO: 16) to permit secretion, as described below.

### EXAMPLE 3

### Generation of the Whole Soluble Spike Protein (sS) Lacking the Cytoplasmic Tail and the Transmembrane Domain

The following pair of primers were used to generate a nucleic acid segment encoding a fragment of the spike protein (sS) lacking the cytoplasmic tail having amino acids 17-1189 of SEQ ID NO: 1: S1 Forward: 5′-AGTC GGATCC GAC CGG TGC ACC ACT TTT G-3′ (SEQ ID NO: 9), and Reverse: 5′ ACTG TCTAGA TTG CTC ATA TTT TCC C-3′ (SEQ ID NO: 12).

### EXAMPLE 4

### Expression of an Amino-Terminal and Carboxyl-Terminal Fragment of a Spike Protein

Expression will be done by transfecting an expression construct containing the pSecTag2B or pCDNA3.1(+) plasmid and a nucleic acid insert that encodes an amino-terminal (S1), a carboxyl-terminal (S2) fragment, or a fragment of the spike protein of SARS-CoV that lacks the cytoplasmic tail and the transmembrane domain, into 293 or Vero E6 cells. It is thought that elimination of the transmembrane domain allows the polypeptides and peptide fragments to be soluble in an aqueous solution. Expression efficiency of the encoded fragments will then be tested. Once a positive signal is obtained as determined with gel analysis, a stably transfected cell line will be generated. The full length spike protein, and fragments thereof will be purified according to methods that are routinely used with other highly glycosylated proteins. Such as use of a lentil lectin column for large production. The resulting proteins: soluble S1 (sS1), soluble S2 (sS2) and whole soluble S (sS) will have the following amino acid sequences. Bold lettering denotes the signal peptide which can be cleaved so the excreted protein will not contain it.

Amino Acid Sequence of a Soluble Amino-Terminal Fragment of the Spike Protein (Amino Acids 17-757)

(SEQ ID NO:13)

DRCTTFDDVQAPNYTQHTSSMRGVYYPDEIFRSDTLYLTQDLFLPFYSNV
TGFHTINHTFGNPVIPFKGIYFAATEKSNVVRGWVFGSTMNNKSQSVIII
NNSTNVVIRACNFELCDNPFFAVSKPMGTQTHTMIFDNAFNCTFEYISDA
FSLDVSEKSGNFKHLREFVFKNKDGFLYVYKGYQPIDVVRDLPSGFNTLK
PIFKLPLGINITNFRAILTAFSPAQDIWGTSAAAYFVGYLKPTTFMLKYD
ENGTITDAVDCSQNPLAELKCSVKSFEIDKGIYQTSNFRVVPSGDVVRFP
NITNLCPFGEVFNATKFPSVYAWERKKISNCVADYSVLYNSTFFSTFKCY
GVSATKLNDLCFSNVYADSFVVKGDDVRQIAPGQTGVIADYNYKLPDDFM
GCVLAWNTRNIDATSTGNYNYKYRYLRHGKLRPFERDISNVPFSPDGKPC
TPPALNGYWPLNDYGFYTTTGIGYQPYRVVVLSFELLNAPATVCGPKLST
DLIKNQCVNIFNIFNGLTGTGVLTPSSKRFQPFQQFGRDVSDFTDSVRDP
KTSEILDISPCAFGGVSVITPGTNASSEVAVLYQDVNCTDVSTAIHADQL
TPAWRIYSTGNNVFQTQAGCLIGAEHVDTSYECDIPIGAGICASYHTVSL
LRSTSQKSIVAYTMSLGADSSLAYSNNTIAIPTNFSISITTEVMPVSMAK
TSVDCNMYICGDSTECANLLLQYGSFCTQLNRALSGIAAEQ

Amino Acid Sequence of a Soluble Carboxyl-Terminal Fragment of the Spike Protein (Amino Acids 762-1189)

(SEQ ID NO: 14)

EVFAQVKQMYKTPTLKYFGGFNFSQILPDPLKPTKRSFIELDLLFNKVTL
ADAGFMKQYGECLGDINARDLICAQKFNGLTVLPPLLTDDMIAAYTAALV
SGTATAGWTFGAGAALQIPFAMQMAYRFNGIGVTQNVLYENQKQIANQFN
KAISQIQESLTTTSTALGKLQDVVNQNAQALNTLVKQLSSNFGAISSVLN
DILSRLDKVEAEVQIDRLITGRLQSLQTYVTQQLIRAAEIRASANLAATK
MSECVLGQSKRVDFCGKGYHLMSFPQAAPHGVVFLHVTYVPSQERNFTTA
PAICHEGKAYFREGVFVFNGTSWFITQRNFFSPQIITTDNTFVSGNCDVV
IGIINNTVYDPLQPELDSFKEELDKYFKNHTSPDVDLGDISGINASVVNI
QKEIDRLNEVAKNLNESLIDLQELGKYEQ

Amino Acid Sequence of a Soluble Spike Protein Having Amino Acids 17-757 and 762-1189 of SEQ ID NO: 1 (Lacking the Signal Peptide and the Potential Cleavage Site)

(SEQ ID NO: 15)

DRCTTFDDVQAPNYTQHTSSMRGVYYPDEIFRSDTLYLTQDLFLPFYSNF
VTGFHTINHTFGNPVIPFKDGIYFAATEKSNVVRGWVFGSTMNNKSQSVI
IINNNSTNVVIRACNFELCDNPFFAVSKPMGTQTHTMIFDNAFNCTFEYI
SDAFSLDVSEKSGNFKHLREFVFKNKDGFLYVYKGYQPIDVVRDLPSGFN
TLKPIFKLPLGINITNFRMLTAFSPAQDIWGTSAAAYFVGYLKPTTFMLK
YDENGTITDAVDCSQNPLAELKCSVKSFEIDKGIYQTSNFRVVPSGDVVR
FPNITNLCPFGEVFNATKFPSVYAWERKKISNCVADYSVLYNSTFFSTFK
CYGVSATKLNDLCFSNVYADSFVVKGDDVRQIAPGQTGVIADYNYKLPDD
FMGCVLAWNTRMDATSTGNYNYKYRYLRHGKLRPFERDISNVPFSPDGKP
CTPPALNCYWPLNDYGFYTFLTGIGYQPYRVVVLSFELLNAPATVCGPKL
STDLIKNQCVNFNFNGLTGTGVLTPSSKRFQPFQQFGRDVSDETDSVRDP
KTSEILDISPCAFGGVSVITPGTNASSEVAVLYQDVNCTDVSTAIHADQL
TPAWRIYSTGNNVFQTQAGCLIGAEHVDTSYECDIPIGAGICASYHTVSL
LRSTSQKSIVAYTMSLGADSSIAYSNNTIAIPTNFSISITTEVMPVSMAK
TSVDCNMYICGDSTECANLLLQYGSFCTQLNRALSGIAAEQDEVFAQVKQ
MYKTPTLKYFGGFNFSQILPDPLKPTKRSFIEDLLFNKVTLADAGFMKQY
GECLGDINARDLICAQKFNGLTVLPPLLTDDMIAAYTAALVSGTATAGWT
FGAGAALQIPFAMQMAYRFNGIGVTQNVLYENQKQIANQFNKAISQIQES
LTTTSTALGKLQDVVNQNAQALNTLVKQLSSNFGAISSVLNDILSRLDKV
EAEVQIDRLITGRLQSLQTYVTQQLIRAAEIRASANLAATKMSECVLGQS
KRVDFCGKGYHLMSFPQAAPHGVVFLHVTYVPSQERNFTTAPAICHEGKA
YFPREGVFVFNGTSWFITQRNFFSPQIITTDNTFVSGNCDVVIGITNNTV
YDPLQPELDSFKEELDKYFKMITSPDVDLGDISGINASVVNIQKEIDRLN
EVAKNLNESLIDLQELGKYEQ

### EXAMPLE 5

### Generation of Additional Soluble Fragments of the Spike Protein

The nucleic acid sequence encoding a polypeptide containing amino acids 17-757 of SEQ ID NO: 1 was obtained through use of polymerase chain reaction (PCR). The following primers were used during the PCR reactions to amplify the nucleic acid sequence: Forward primer: 5′ AGCT GGA TCC GAC CGG TGC ACC ACT TTT G 3′ (SEQ ID NO: 9); and Reverse primer: 5′ AGCT GGG CCC CTG TTC AGC AGC AAT ACC 3′ (SEQ ID NO: 10). The resulting PCR product was digested with BamHI and ApaI and, encodes a polypeptide having an amino acid sequence corresponding to SEQ ID NO: 43. The digested PCR product was then ligated to pSecTag2B (Invitrogen, Carlsbad, Calif.) that was digested with the same enzymes. The pSecTag2B construct containing the PCR product insert encodes a polypeptide having SEQ ID NO: 46 with the mouse k chain leader sequence (METDTLLLWVLLLWVPGSTGD) (SEQ ID NO: 16) at the N-terminus for secretion, and a myc epitope (EQKLISEEDL) (SEQ ID NO: 17) plus a histidine tag (HHHHHH) (SEQ ID NO: 18) at the C-terminus for affinity purification.

The nucleic acid sequence encoding a polypeptide containing amino acids 17-276 of SEQ ID NO: 1 was obtained through use of polymerase chain reaction (PCR). The following primers were used during the PCR reactions to amplify the nucleic acid sequence: Forward primer: 5′ AGCT GGA TCC GAC CGG TGC ACC ACT TTT G 3′ (SEQ ID NO: 9); and Reverse primer: 5′ CTAG CTC GAG CAA CAG CAT CTG TG 3′ (SEQ ID NO: 19). The resulting PCR product was digested with BamHI and XhoI and, encodes an amino acid having SEQ ID NO: 44. The digested PCR product was then ligated to pSecTag2B (Invitrogen, Carlsbad, Calif.) that was digested with the same enzymes. The pSecTag2B construct containing the PCR product insert encodes a polypeptide having SEQ ID NO: 47 with the mouse k chain leader sequence (METDTLLLWVLLLWVPGSTGD) (SEQ ID NO: 16) at the N-terminus for secretion, and a myc epitope (EQKLISEEDL) (SEQ ID NO: 17) plus a histidine tag (HHHHHH) (SEQ ID NO: 18) at the C-terminus for affinity purification.

The nucleic acid sequence encoding a polypeptide containing amino acids 17-537 of SEQ ID NO: 1 was obtained by digesting the nucleic acid sequence that encodes SEQ ID NO: 43 (as described above) with BamHI and HincII. The nucleic acid segment produced encodes a polypeptide having SEQ ID NO: 45. This nucleic acid segment was ligated into a pSecTag2B vector that was digested with BamHI and EcoRV. The pSecTag2B construct containing the PCR product insert encodes a polypeptide having SEQ ID NO: 48 with the mouse k chain leader sequence (METDTLLLWVLLLWVPGSTGD) (SEQ ID NO: 16) at the N-terminus for secretion, and a myc epitope (EQKLISEEDL) (SEQ ID NO: 17) plus a histidine tag (HHHHH) (SEQ ID NO: 18) at the C-terminus for affinity purification.

The expression of these peptide fragments in mammalian cells is illustrated in FIG. 3. This figure shows that the peptide fragments can be secreted into medium in which cells that express the peptide fragments are grown. FIG. 3 also indicates that the peptide fragments are soluble in aqueous medium.

TABLE 1

Examples of additional peptide fragments
of the invention

| | | |
|---|---|---|
| | Amino Acid | Amino Acid Sequence |
| | Position | Amino acid residues in bold |
| SEQ ID | Relative to | (amino acid residues 1-16) |
| NUMBER | SEQ ID NO: 1 | identify a signal sequence. |

| | | |
|---|---|---|
| 20 | 1-100 | MFIFLLFLTLTSGSDLDRCTTFDDVQP |
| | | NYTQHTSSMRGVYYPDEIFRSDTLYLT |
| | | QDLFLPFYSNVTGFHTINHTFGNPVIP |
| | | FKDGIYFAATEKSNVVRG |
| 21 | 101-200 | WVFGSTMNNKSQSVIIINNSTNVVIRA |
| | | CNFELCDNPFFAVSKPMGTQTHTMIFD |
| | | NAFNCTFEYISDAFSLDVSEKSGNFKH |
| | | LREFVFKNKDGFLYVYKGY |
| 22 | 201-300 | QPIDVVRDLPSGFNTLKPIFKLPLGIN |
| | | ITNFRAILTAFSPAQDIWGTSAAAYFV |
| | | GYLKPTTFMLKYDENGTITDAVDCSQN |
| | | PLAELKCSVKSFEIDKGIY |
| 23 | 301-400 | QTSNFRVVPSGDVVRFPNITNLCPFGE |
| | | VFNATKFPSVYAWERKKISNCVADYSV |
| | | LYNSTFFSTFKCYGVSATKLNDLCFSN |
| | | VYADSFVVKGDDVRQIAPG |
| 24 | 401-500 | QTGVIADYNYKLPDDFMGCVLAWNTRN |
| | | IDATSTGNYNYKYRYLRHGKLRPFERD |
| | | ISNVPFSPDGKPCTPPALNCYWPLNDY |
| | | GFYTITTGIGYQPYRVVVLS |
| 25 | 501-600 | FELLNAPATVCGPKLSTDLIKNQCVNF |
| | | NFNGLTGTGVLTPSSKRFQPFQQFGRD |
| | | VSDFTDSVRDPKTSEILDISPCAFGGV |
| | | SVITPGTNASSEVAVLYQD |
| 26 | 601-700 | VNCTDVSTAIHADQLTPAWRIYSTGNN |
| | | VFQTQAGCLIGAEHVDTSYECDIPIGA |
| | | GICASYHTVSLLRSTSQKSIVAYTMSL |
| | | GADSSIAYSNNTIAIPTNF |
| 27 | 701-800 | SISITTEVMPVSMAKTSVDCNMYICGD |
| | | STECANLLLQYGSFCTQLNRALSGIAA |
| | | EQDRNTREVFAQVKQMYKTPTLKYFGG |
| | | FNFSQILPDPLKPTKRSFI |
| 28 | 801-900 | EDLLFNKVTLADAGFMKQYGECLGDIN |
| | | ARDLICAQKFNGLTVLPPLLTDDMIAA |
| | | YTAALVSGTATAGWTFGAGAALQIPFA |
| | | MQMAYRFNGIGVTQNVLYE |
| 29 | 901-1000 | NQKQIANQFNKAISQIQESLTTTSTAL |
| | | GKLQDVVNQNAQALNTLVKQLSSNFGA |
| | | ISSVLNDILSRLDKVEAEVQIDRLITG |
| | | RLQSLQTYVTQQLIRAAEI |
| 30 | 1001-1100 | RASANLAATKMSECVLGQSKRVDFCGK |
| | | GYHLMSFPQAAPHGVVFLHVTYVPSQE |
| | | RNFTTAPAICHEGKAYFPREGVFVFNG |
| | | TSWFITQRNFFSPQIITTD |
| 31 | 1101-1189 | NTFVSGNCDVVIGIINNTVYDPLQPEL |
| | | DSFKEELDKYFKNHTSPDVDLGDISGI |
| | | NASVVNIQKEIDRLNEVAKNLNESLID |
| | | LQELGKYEQ |
| 32 | 1-200 | MFIFLLFLTLTSGSDLDRCTTFDDVQA |
| | | PNYTQHTSSMRGVYYPDEIIFRSDTLY |
| | | LTQDLFLPFYSNVTGFHTINHTFGNPV |
| | | IPFKDGIYFAATEKSNVVRGWVFGSTM |
| | | NNKSQSVIINNSTNVVIRACNFELCDN |
| | | PFFAVSKPMGTQTHTMIFDNAFNCTFE |
| | | YISDAFSLDVSEKSGNFKHLREFVFKN |
| | | KDGFLYVYKGY |
| 33 | 201-400 | QPIDVVRDLPSGFNTLKPIFKLPLGIM |
| | | TNFRAILTAESPAQDIWGTSAAAYFVG |
| | | YLKPTTFMLKYDENGTITDAVDCSQNP |
| | | LAELKCSVKSFEIDKGIYQTSNFRVVP |
| | | SGDVVRFPNITNLCPFGEVFNATKFPS |
| | | VYAWERKKISNCVADYSVLYNSTFFST |
| | | FKCYGVSATKLNDLCFSNVYADSFVVK |
| | | GDDVRQIAPG |
| 34 | 401-600 | QTGVIADYNYKLPDDFMGCVLAWNTRN |
| | | IDATSTGNYNYKYRYLRHGKLRPFERD |
| | | ISNVPFSPDGKPCTPPALNCYWPLNDY |
| | | GFYITTGIGYQPYRVVVLSFELLNAPA |
| | | TVCGPKLSTDLIKNQCVNFNFNGLTGT |
| | | GVLTPSSKRFQPFQQFGRDVSDFTDSV |
| | | RDPKTSEILDISPCAFGGVSVITPGTN |
| | | ASSEVAVLYQD |
| 35 | 601-800 | VNCTDVSTAIHADQLTPAWRIYSTGNN |
| | | VFQTQAGCLIGAEHVDTSYECDIPIGA |
| | | GICASYHTVSLLRSTSQKSIVAYTMSL |
| | | GADSSIAYSNNTIAIPTNFSISITTEV |
| | | MIPVSMAKTSVDCNMYICGDSTECANL |
| | | LLQYGSFCTQLNRALSGIAAEQDRNTR |
| | | EVFAQVKQMYKTPTLKYFGGFNFSQIL |
| | | PDPLKPTKRSFI |
| 36 | 801-1000 | EDLLFNKVTLADAGFMKQYGECLGDIN |
| | | ARDLICAQKFNGLTVLPPLLTDDMIIA |
| | | AYTAALVSGTATAGWTFGAGAALQIPF |
| | | AMQMAYRFNGIGVTQNVLYENQKQIAN |
| | | QFNKAISQIQESLITTSTALGKLQDVV |
| | | NQNAQALNTLVKQLSSNFGAISSVLND |
| | | ILSRLDKVEAEVQIDRLITGRLQSLQT |
| | | YVTQQLIRAAEI |
| 37 | 1001-1189 | RASANLAATKMSECVLGQSKRVDFCGK |
| | | GYHLMSFPQAAPHGVVFLHVTYVPSQE |
| | | RNFTTAPAICHEGKAYFPREGVFVFNG |
| | | TSWFITQRNFFSPQIITTDNTFVSGNC |
| | | DVVIGHNNTVYDPLQPELDSFKEELDK |
| | | YFKNHTSPDVDLGDISGIINASVVNIQ |
| | | KEIDRLNEVAKNLNESLIDLQELGKYE |
| | | Q |
| 38 | 1-400 | MFIFLLFLTLTSGSDLDRCTIFDDVQA |
| | | PNYTQHTSSMRGVYYPDEIFRSDTLYL |
| | | TQDLFLPFYSNVTGFHTINHTFGNPVI |
| | | PFKDGIYFAATEKSNVVRGWVFGSTMN |
| | | NKSQSVIIINNSTNVVIRACNFELCDN |
| | | PFFAVSKPMGTQTHTMIFDNAFNCTFE |
| | | YISDAFSLDVSEKSGNFKHLREFVFKN |
| | | KDGFLYVYKGYQPIDVVRDLPSGFNTL |
| | | KPIFKLPLGIMTNFRAILTAFSPAQDI |
| | | WGTSAAAYFVGYLKPTTFMLKYDENGT |
| | | ITDAVDCSQNPLAELKCSVKSFEIDKG |
| | | IYQTSNFRVVPSGDVVRFPNITNLCPF |
| | | GEVFNATKFPSVYAWERKKISNCVADY |
| | | SVLYNSTFFSTFKCYGVSATKLNDLCF |
| | | SNVYADSFVVKGDDVRQIAPG |
| 39 | 1-600 | MFIFLLFLTLTSGSDLDRCTTFDDVQA |
| | | PNYTQHTSSMRGVYYPDEIFRSDTLYL |
| | | TQDLFLPFYSNVTGFHTINHTFGNPVI |
| | | PFKDGIYFAATEKSNVVRGWVFGSTMN |
| | | NKSQSVIIINNSTNVVIRACNFELCDN |
| | | PFFAVSKPMGTQTHTMIFDNAFNCTFE |
| | | YISDAFSLDVSEKSGNFKHLREFVFKN |
| | | KDGFLYVYKGYQPIDVVRDLPSGFNTL |
| | | KPIFKLPLGINITNFRAILTAFSPAQD |
| | | IWGTSAAAYFVGYLKPITFMLKYDENG |
| | | TITDAVDCSQNPLAELKCSVKSFEIDK |
| | | GIYQTSNFRVVPSGDVVRFPNITNLCP |
| | | FGEVFNATKFPSVYAWERKKISNCVAD |
| | | YSVLYNSTFFSTFKCYGVSATKLNDLC |
| | | FSNVYADSFVVKGDDVRQIAPGQTGVI |
| | | ADYNYKLPDDFMGCVLAWNTRNIDATS |
| | | TGNYNYKYRYLRHGKLRPFERDISNVP |
| | | FSPDGKPCTPPALNCYWPLNDYGFYTT |
| | | TGIGYQPYRVVVLSFELLNAPATVCGP |
| | | KLSTDLIKNQCVNFNFNGLTGTGVLTP |
| | | SSKRFQPFQQFGRDVSDFTDSVRDPKT |
| | | SEILDISPCAFGGVSVITPGTNASSEV |
| | | AVLYQD |
| 40 | 1-800 | MFIFLLFLTLTSGSDLDRCTIFDDVQA |
| | | PNYTQHTSSMRGVYYPDEIFRSDTLYL |
| | | TQDLFLPFYSNVTGFHTINHTFGNIPV |
| | | IPFKDGIYFAATEKSNVVRGWVFGSTM |
| | | NNKSQSVIIINNSTNVVIRACNFELCD |
| | | NPFFAVSKPMGTQTHTMIFDNAFNCTF |
| | | EYISDAFSLDVSEKSGNFKIILREFVF |
| | | KNKDGFLYVYKGYQPIDVVRDLPSGFN |
| | | TLKPIFKLPLGINITNFRAILTAFSPA |
| | | QDIWGTSAAAYFVGYLKPTTFMLKYDE |
| | | NGTITDAVDCSQNPLAELKCSVKSFEI |
| | | DKGIYQTSNFRVVPSGDVVRFPNITNL |
| | | CPFGEVFNATKFPSVYAWERKKISNCV |
| | | ADYSVLYNSTFFSTFKCYGVSATKLND |
| | | LCFSNVYADSFVVKGDDVRQIAPGQTG |
| | | VIADYNYKLPDDFMGCVLAWNTRNIDA |
| | | TSTGNYNYKYRYLRHGKLRPFERDISN |
| | | VPFSPDGKPCTPPALNCYWPLNDYGFY |
| | | TTTGIGYQPYRVVVLSFELLNAPATVC |
| | | GPKLSTDLIKNQCVNFNFNGLTGTGVL |
| | | TPSSKRFQPFQQFGRDVSDFTDSVRDP |
| | | KTSEILDISPCAFGGVSVITPGTNASS |
| | | EVAVLYQDVNCTDVSTAIHADQLTPAW |
| | | RIYSTGNNVFQTQAGCLIGAEHVDTSY |
| | | ECDIPIGAGICASYHTVSLLRSTSQKS |
| | | IVAYTMSLGADSSIAYSNNTIAIPTNI |
| | | FSISITITEVMPVSMAKTSVDCNMYIC |
| | | GDSTECANLLLQYGSFCTQLNRALSGI |
| | | AAEQDRNTREVFAQVKQMYKTPTLKYF |
| | | GGFNFSQILPDPLKPTKRSFI |
| 41 | 1-1000 | MFIFLLFLTLTSGSDLDRCTTFDDVQA |
| | | PNYTQHTSSMRGVYYPDEIFRSDTLYL |
| | | TQDLFLPFYSNVTGFHTINHTFGNPVI |
| | | PFKDGIYFAATEKSNYVRGWVFGSTMN |
| | | NKSQSVIIINNSTNVVIRACNFELCDN |
| | | PFFAVSKPMGTQTHTMIFDNAFNCTFE |
| | | YISDAFSLDVSEKSGNFKHLREFVFKN |
| | | KDGFLYVYKGYQPIDVVRDLPSGFNTL |
| | | KPIFKLPLGINITNFRAILTAFSPAQD |
| | | IWGTSAAAYFVGYLDPTTFMLKYDENG |
| | | TITDAVDCSQNPLAELKCSVKSFEIDK |
| | | GIYQTSNFRVVPSGDVVRFPNITNLCP |
| | | FGEVFNATKFPSVYAWERKKISNCVAD |
| | | YSVLYNSTFFSTFKCYGVSATKLNDLC |
| | | FSNVYADSFVVKGDDVRQIAPGQTGVI |
| | | ADYNYKLPDDFMGCVLAWNTRNIDATS |
| | | TGNYNYKYRYLRHGKLRPFERDISNVP |
| | | FSPDGKPCTPPALNCYWPLNDYGFYTT |
| | | GIGYQPYRVVVLSFELLNAPATVCGPK |
| | | LSTDLIKNQCVNFNFNGLTGTGVLTPS |
| | | SKRFQPFQQFGRDVSDFTDSVRDPKTS |
| | | EILDISPCAFGGVSVITPGTNASSEVA |
| | | VLYQDVNCTDVSTAIHADQLTPAWRIY |
| | | STGNNVFQTQAGCLIGAEHVDTSYECD |
| | | IPIGAGICASYHTVSLLRSTSQKSIVA |
| | | YTMSLGADSSIAYSNNTIAIPTNFSIS |
| | | ITTEVMPVSMAKTSVDCNMYICGDSTE |
| | | CANLLLQYGSFCTQLNRALSGIAAEQD |
| | | RNTREVFAQVKQMYKTPTLKYFGGFNF |
| | | SQILPDPLKPTKRSFIEDLLFNKVTLA |
| | | DAGFMKQYGECLGDINARDLICAQKFN |
| | | GLTVLPPLLTDDMIAAYTAALVSGTAT |
| | | AGWTFGAGAALQIPFAMQMAYRFNGIG |
| | | VTQNVLYENQKQIANQFNKAISQIQES |
| | | LTTTSTALGKLQDVVNQNAQALNTLVK |
| | | QLSSNFGAISSVLNDILSRLDKVEAEV |
| | | QIDRLITGRLQSLQTYVTQQLIRAAEI |
| 42 | 1-1189 | MFIFLLFLTLTSGSDLDRCTTFDDVQA |
| | | PNYTQHTSSMRGVYYPDEIFRSDTLYL |
| | | TQDLFLPFYSNVTGFHTINHTFGNPVI |
| | | PFKDGIYFAATEKSNVVRGWVFGSTMI |
| | | NNKSQSVIILNNSTNVVIRACNFELCD |
| | | NPFFAVSKPMGTQTHTMIFDNAFNCTF |
| | | EYISDAFSLDVSEKSGNFKHLREFVFK |
| | | NKDGFLYVYKGYQPIDVVRDLPSGFNT |
| | | LKPIFKLPLGLNITNFRAILTAFSPAQ |
| | | DIWGTSAAAYFVGYLKPTTFMLKYDEN |
| | | GTITDAVDCSQNPLAELKCSVKSFEID |
| | | KGIYQTSNFRVVPSGDVVRFPNITNLC |
| | | PFGEVFNATKFPSVYAWERKKISNCVA |
| | | DYSVLYNSTFFSTFKCYGVSATKLNDL |
| | | CFSNVYADSFVVKGDDVRQIAPGQTGV |
| | | IADYNYKLPDDFMGCVLAWNTRNIDAT |
| | | STGNYNYKYRYLRHGKLRPFERDISNV |
| | | PFSPDGKPCTPPALNCYWPLNDYGFYT |
| | | TTGIGYQPYRVVVLSFELLNAPATVCG |
| | | PKLSTDLIKNQCVNFNFNGLTGTGVLT |
| | | PSSKRFQPFQQFGRDVSDFTDSVRDPK |
| | | TSEILDISPCAFGGVSVITPGTNASSE |
| | | VAVLYQDVNCTDVSTAIHADQLTPAWR |
| | | IYSTGNNVFQTQAGCLIGAEHVDTSYE |
| | | CDIPIGAGICASYHTVSLLRSTSQKSI |
| | | VAYTMSLGADSSIAYSNNTIAIPTNFS |
| | | ISVFITEVMPVSMAKTSVDCNMYICGD |
| | | STECANLLLQYGSFCTQLNRALSGLAA |
| | | EQDRNTREVFAQVKQMYKTPTLKYFGG |
| | | FNFSQILPDPLKPTKRSFIEDLLFNKV |
| | | TLADAGFMKQYGECLGDINARDLICAQ |
| | | KFNGLTVLPPLLTDDMIAAYTAALVSG |
| | | TATAGWTFGAGAALQIPFAMQMAYRFN |
| | | GIGVTQNVLYENQKQIANQFNKAISQI |
| | | QESLTTTSTALGKLQDVVNQNAQALNT |
| | | LVKQLSSNFGAISSVLNDILSRLDKVE |
| | | AEVQIDRLITGRLQSLQTYVTQQLIRA |
| | | AEIRASANLAATKMSECVLGQSKRVDF |
| | | CGKGYHLMSFPQAAPHGVVFLHVTYVP |
| | | SQERNFTTAPAICHEGKAYFPREGVFV |
| | | FNGTSWFITQRNFFSPQIITTDNTFVS |
| | | GNCDVVIGIIINNTVYDPLQPELDSFK |
| | | EELDKYFKNHTSPDVDLGDISGINASV |
| | | VNIQKEIDRLNEVAKNLNESLIDLQEL |
| | | GKYEQ |
| 43 | 17-100 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRG |
| 44 | 17-200 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELCDNPFFAVSKPMGTQTHTM |
| | | IFDNAFNCTFEYISDAFSLDVSEKSGN |
| | | FKHLREFVFKNKDGFLYVYKGY |
| 45 | 17-400 | DRCTITFDDVQAPNYTQHTSSMRGVYY |
| | | PDEIFRSDTLYLTQDLFLPFYSNVTGF |
| | | HTINHTFGNPVIPFKDGIYFAATEKSN |
| | | VVRGWVFGSTMNNKSQSVIIINNSTNV |
| | | VIRACNFELCDNPFFAVSKPMGTQTHT |
| | | MIFDNAFNCTFEYISDAFSLDVSEKSG |
| | | NFKLHLREFVFKNKDGFLYVYKGYQPI |
| | | DVVRDLPSGFNTLKPIFKLPLGINITN |
| | | FRAILTAFSPAQDIWGTSAAAYFVGYL |
| | | KPTTFMLKYDENGTITDAVDCSQNPLA |
| | | ELKCSVKSFEIDKGIYQTSNFRVVPSG |
| | | DVVRFPNITNLCPFGEVFNATKFPSVY |
| | | AWERKKISNCVADYSVLYNSTFFSTFK |
| | | CYGVSATKLNDLCFSNVYADSFVVKGD |
| | | DVRQIAPG |
| 46 | 17-600 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELCDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGINITNFR |
| | | AILTAFSPAQDIWGTSAAAYFVGYLKP |
| | | TTFMLKYDENGTITDAVDCSQNPLAEL |
| | | KCSVKSFEIDKGIYQTSNFRVVPSGDV |
| | | VRFPNITNLCPFGEVFNATKFPSVYAW |
| | | ERKKISNCVADYSVLYNSTFFSTFKCY |
| | | GVSATKLNDLCFSNVYADSFVVKGDDV |
| | | RQIAPGQTGVIADYNYKLPDDFMGCVL |
| | | AWNTRNIDATSTGNYNYKYRYLRHGKL |
| | | RPFERDISNVPFSPDGKPCTPPALNCY |
| | | WPLNDYGFYTTTGIGYQPYRVVVLSFE |
| | | LLNAPATVCGPKLSTDLIKNQCVNFNF |
| | | NGLTGTGVLTPSSKRFQPFQQFGRDVS |
| | | DFTDSVRDPKTSEILDISPCAFGGVSV |
| | | VITPGTNASSEVAVLYQD |
| 47 | 17-800 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIINNSTNVVI |
| | | RACNFELCDNPFFAVSKPMGTQTHTMI |
| | | FDNAFNCTFEYISDAFSLDVSEKSGNF |
| | | KHLREFVFKNKDGFLYVYKGYQPIDVV |
| | | RDLPSGFNTLKPIFKLPLGIMTNFRAI |
| | | LTAFSPAQDIWGTSAAAYFVGYLKPTT |
| | | FMLKYDENGTITDAVDCSQNIPLAELK |
| | | CSVKSFEIDKGIYQTSNFRVVPSGDVV |
| | | RFPNITNLCPFGEVFNATKFPSVYAWE |
| | | RKKISNCVADYSVLYNSTFFSTFKCYG |
| | | VSATKLNDLCFSNVYADSFVVKGDDVR |
| | | QIAPGQTGVIADYNYKLPDDFMGCVLA |
| | | WNTRNIDATSTGNYNYKYRYLRHGKLR |
| | | PFERDISNVPFSPDGKIPCTPPALNCY |
| | | WPLNDYGFYTTTGIGYQPYRVVVLSFE |
| | | LLNAPATVCGPKLSTDLIKNQCVNFNF |
| | | NGLTGTGVLTPSSKRFQPFQQFGRDVS |
| | | DFTDSVRDPKTSEILDISPCAFGGVSV |
| | | ITPGTNASSEVAVLYQDVNCTDVSTAI |
| | | HADQLTPAWRIYSTGNNVFQTQAGCLI |
| | | GAEHVDTSYECDIPIGAGICASYHTVS |
| | | LLRSTSQKSIVAYTMSLGADSSIAYSN |
| | | NTIAIPTNFSISITITEVMPVSMAKTS |
| | | VDCNMYICGDSTECANLLLQYGSFCTQ |
| | | LNRALSGIAAEQDRNTREVFAQVKQMY |
| | | KTPTLKYFGGFNFSQILPDPLKPTKRS |
| | | FI |
| 48 | 17-1000 | DRGITFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELCDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGIMTNFRA |
| | | ILTAFSPAQDIWGTSAAAYFVGYLKPT |
| | | TFMLKYDENGTITDAVDCSQNPLAELK |
| | | CSVKSFEIDKGIYQTSNFRVVPSGDVV |
| | | RFPNITNLCPFGEVFNATKFPSVYAWE |
| | | RKKISNCVADYSVLYNSTFFSTFKCYG |
| | | VSATKLNDLCFSNVYADSFVVKGDDVR |
| | | QIAPGQTGVIADYNYKLPDDFMGCVLA |
| | | WNTRNIDATSTGNYNYKYRYLRHGKLR |
| | | PFERDISNVPFSPDGKPCTPPALNCYW |
| | | PLNDYGFYTLTGIGYQPYRVVVLSFEL |
| | | LNAPATVCGPKLSTDLIKNQCVNIFNF |
| | | NGLTGTGVLTPSSKRFQPFQQFGRDVS |
| | | DFTDSVRDPKTSEILDISPCAFGGVSV |
| | | ITPGTNASSEVAVLYQDVNCTDVSTAI |
| | | HADQLTPAWRIYSTGNNVFQTQAGCLI |
| | | GAEHVDTSYECDIPIGAGICASYHTVS |
| | | LLRSTSQKSIVAYTMSLGADSSIAYSN |
| | | NTIAIPTNFSISITTEVMPVSMAKTSV |
| | | DCNMYICGDSTECANLLLQYGSFCTQL |
| | | NRALSGIAAEQDRNTREVFAQVKQMYK |
| | | TPTLKYFGGFNFSQILPDPLKPTKRSF |
| | | IEDLLFNKVTLADAGFMKQYGECLGDI |
| | | NARDLICAQKFNGLTVLPPLLTDDMAA |
| | | YTAALVSGTATAGWTFGAGAALQIPFA |
| | | MQMAYRFNGIGVTQNVLYENQKQIANQ |
| | | FNKAISQIQESLTTTSTALGKLQDVVN |
| | | QNAQALNTLVKQLSSNFGAISSVLNDI |
| | | LSRLDKVEAEVQIDRLITGRLQSLQTY |
| | | VTQQLIRAAEI |
| 49 | 17-1189 | DRCTTFDDVQAPNYTQHTSSMRGVYYP |
| | | DELFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELCDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGIMTNFRA |
| | | ILTAFSPAQDIWGTSAAAYFVGYLKPT |
| | | TFMLKYDENGTITDAVDCSQNPLAELK |
| | | CSVKSFEIDKGLYQTSNFRVVPSGDVV |
| | | RFPNITNLCPFGEVFNATKFPSVYAWE |
| | | RKKISNCVADYSVLYNSTFFSTFKCYG |
| | | VSATKLNDLCFSNVYADSFVVKGDDVR |
| | | QIAPGQTGVIADYNYKLPDDFMGCVLA |
| | | WNTRNIDATSTGNYNYKYRYLRHGKLR |
| | | PFERDISNVPFSPDGKPCTPPALNCYW |
| | | PLNDYGFYTTTGIGYQPYRVVVLSFEL |
| | | LNAPATVCGPKLSTDLIKNQCVNFNFN |
| | | GLTGTGVLTPSSKRFQPFQQFGRDVSD |
| | | FTDSVRDPKTSEILDISPCAFGGVSVI |
| | | TPGTNASSEVAVLYQDVNCTDVSTAIH |
| | | ADQLTPAWRIYSTGNNVFQTQAGCLIG |
| | | AEHVDTSYECDIPIGAGICASYHTVSL |
| | | LRSTSQKSIVAYTMSLGADSSIAYSNN |
| | | TIAIPTNFSISITTEVMPVSMAKTSVD |
| | | CNMYICGDSTECANLLLQYGSFCTQLN |
| | | RALSGIAAEQDRNTREVFAQVKQMYKT |
| | | PTLKYFGGFNFSQILPDPLKPTKRSFI |
| | | EDLLFNKVTLADAGFMKQYGECLGDIN |
| | | ARDLICAQKFNGLTVLPPLLTDDMIAA |
| | | YTAALVSGTATAGWTFGAGAALQIPFA |
| | | MQMAYRFNGIGVTQNVLYENQKQIANQ |
| | | FNKAISQIQESLTTTSTALGKLQDVVN |
| | | QNAQALNTLVKQLSSNFGAISSVLNPI |
| | | LSRLDKVEAEVQIDRLITGRLQSLQTY |
| | | VTQQLIRAAEIRASANLAATKMSECVL |
| | | GQSKRVDFCGKGYHLMSFPQAAPHGVV |
| | | FLHVTYVPSQERNFTTAPAICHEGKAY |
| | | FPREGVFVFNGTSWFITQRNFFSPQII |
| | | TTDNTFVSGNCDVVIGIINNTVYDPLQ |
| | | PELDSFKEELDKYFKNIITSPDVDLGD |
| | | ISGINASVVNIQKEIDRLNEVAKNLNE |
| | | SLIDLQELGKYEQ |
| 50 | 17-276 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELGDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGINITNFR |
| | | AILTAFSPAQDIWGTSAAAYFVGYLKP |
| | | TTFMLKYDENGTITDAV |
| 51 | 17-446 | DRCTTFDDVQAPNYTQHTSSMRGVYYP |
| | | DEIFRSDTLYLTQDLFLPFYSNVTGFH |
| | | TINHTFGNIPVIPFKDGIYFAATEKSN |
| | | VVRGWVFGSTMNNKSQSVIIINNSTNV |
| | | VIRACNFELCDNPFFAVSKPMGTQTHT |
| | | MIFDNAFNCTFEYISDAFSLDVSEKSG |
| | | NFKHLREFVFKNKDGFLYVYKGYQPID |
| | | VVRDLPSGFNTLKPIFKLPLGIMTNFR |
| | | AILTAFSPAQDIWGTSAAAYFVGYLKP |
| | | TTFMLKYDENGTITDAVDCSQNPLAEL |
| | | KCSVKSFEIDKGIYQTSNFRVVPSGDV |
| | | VRFPNITNLCPFGEVFNATKFPSVYAW |
| | | ERKKISNCVADYSVLYNSTFFSTFKCY |
| | | GVSATKLNDLCFSNVYADSFVVKGDDV |
| | | RQIAPGQTGVIADYNYKLPDDFMGCVL |
| | | AWNTRNIDATSTGNYNYKYRYLRHG |
| 52 | 17-537 | DRCTTFDDVQAPNYTQHTSSMRGVYYP |
| | | DEIFRSDTLYLTQDLFLPFYSNVTGFH |
| | | TINHTFGNPVIPFKDGIYFAATEKSNV |
| | | VRGWVFGSTMNNKSQSVIIINNSTNVV |
| | | IRACNFELCDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGINITNFR |
| | | AILTAFSPAQDIWGTSAAAYFVGYLKP |
| | | TTFMLKYDENGTITDAVDCSQNPLAEL |
| | | KCSVKSFEIDKGIYQTSNFRVVPSGDV |
| | | VRFPNITNLCPFGEVFNATKFPSVYAW |
| | | ERKKISNCVADYSVLYNSTFFSTFKCY |
| | | GVSATKLNDLCFSNVYADSFVVKGDDV |
| | | RQIAPGQTGVLADYNYKLPDDFMGCVL |
| | | AWNTRMDATSTGNYNYKYRYLRHGKLR |
| | | PFERDISNVPFSPDGKPCTPPALNCYW |
| | | PLNDYGFYTTTGIGYQPYRVVVLSFEL |
| | | LNAPATVCGPKLSTDLIKNQCVNFNFN |
| | | GLTGTGV |
| 53 | 17-757 plus an | MIETDTLLLWVLLLWVPGSTGDDRCTT |
| | N-terminal | FDDVQAPNYTQHTSSMRGVYYPDEIFR |
| | mouse K chain | SDTLYLTQDLFLPFYSNVTGFHTINHT |
| | leader sequence | FGNPVIPFKDGIYFAATEKSNVVRGWV |
| | and a C- | FGSTMNNKSQSVIIINNSTNVVIRACN |
| | terminal myc | FELCDNPFFAVSKPMGTQTHTMIFDNA |
| | epitope and a | FNCTFEYISDAFSLDVSEKSGNEKHLR |
| | poly histidine | EFVFKNKDGFLYVYKGYQPIDVVRDLP |
| | tag | SGFNTLKIPIFKLPLGINITNFRAILT |
| | | AFSPAQDIWGTSAAAYFVGYLKPTIFM |
| | | LKYDENGTITDAVDCSQNPLAELKCSV |
| | | KSFEIDKGIYQTSNFRVVPSGDVVRFP |
| | | NITNLCPFGEVFNATKFPSVYAWERKK |
| | | ISNCVADYSVLYNSTFFSTFKCYGVSA |
| | | TKLNDLCFSNYYADSFVVKGDDVRQIA |
| | | PGQTGVIADYNYKLPDDFMGCVLAWNT |
| | | RNIDATSTGNYNYKYRYLRHGKLRPFE |
| | | RDISNVPFSPDGKIPCTPPALNCYWPL |
| | | NDYGFYTTTGIGYQPYRVVVLSFELLN |
| | | APATVCGPKLSTDLIKNQCVNFNFNGL |
| | | TGTGVLTPSSKRFQPFQQFGRDVSDFT |
| | | DSVRDPKTSEILDISPCAFGGVSVITP |
| | | GTNASSEVAVLYQDVNCTDVSTAIHAD |
| | | QLTPAWRIYSTGNNVFQTQAGCLIGAE |
| | | HVDTSYECDIPIGAGICASYHTVSLLR |
| | | STSQKSIVAYTMSLGADSSIAYSNNTI |
| | | AIPTNFSISITTEVMPVSMAKTSVDCN |
| | | MYICGDSTECANLLLQYGSFCTQLNRA |
| | | LSGIAAEQEQKLISEEDLHHHHHH |
| 54 | 17-276 plus an | METDTLLLWVLLLWVPGSTGDDRCTTF |
| | N-terminal | DDVQAPNYTQHTSSMRGVYYPDEIFRS |
| | mouse K chain | DTLYLTQDLFLPFYSNVTGFHTINHTF |
| | leader sequence | GNPVIPFKDGIYFAATEKSNVVRGWVF |
| | and a C- | GSTMNNKSQSVIIINNSTNVVIRACNF |
| | terminal myc | ELCDNPFFAVSKPMGTQTHTMIFDNAF |
| | epitope and a | NCTFEYISDAFSLDVSEKSGNFKHLRE |
| | poly histidine | FVFKNKDGFLYVYKGYQPIDVVRDLPS |
| | tag | GFNTLKPIFKLPLGINITNFRAILTAF |
| | | SPAQDIWGTSAAAYFVGYLKPTTFMLK |
| | | YDENGTITDAVEQKLISEEDLHHHHHH |
| 55 | 17-537 plus an | METDTLLLWVLLLWVPGSTGDDRCTTF |
| | N-terminal | DDVQAPNYTQHTSSMRGVYYPDEIFRS |
| | mouse K chain | DTLYLTQDLFLPFYSNVTGFHTINHTF |
| | leader sequence | GNPVIPFKDGIYFAATEKSNVVRGWVF |
| | and a C- | GSTMNNKSQSVIIINNSTNVVIRACNF |
| | terminal myc | ELCDNPFFAVSKPMGTQTHTMIFDNAF |
| | epitope and a | NCTFEYISDAFSLDVSEKSGNFKHLRE |
| | poly histidine | FVFKNKDGFLYVYKGYQPIDVVRDLPS |
| | tag | GFNTLKPIFKLPLGINITNFRAILTAF |
| | | SPAQDIWGTSAAAYFVGYLKPTTFMLK |
| | | YDENGTITDAVDCSQNPLAELKCSVKS |
| | | FEIDKGIYQTSNFRVVPSGDVVRFPNI |
| | | TNLCPFGEVFNATKFPSVYAWERKKIS |
| | | NCVADYSVLYNSTFFSTFKCYGVSATK |
| | | LNDLCFSNVYADSFVVKGDDVRQIAPG |
| | | QTGVIADYNYKLPDDFMGCVLAWNTRN |
| | | IDATSTGNYNYKYRYLRHGKLRPFERD |
| | | ISNVPFSPDGKPGTPPALNCYWPLNDY |
| | | GFYTTTGIGYQPYRVVVLSFELLNAPA |
| | | TVCGPKLSTDLIKNQCVNFNFNGLTGT |
| | | GVEQKLISEEDLHHHHHH |
| 56 | 17-756 | DRCTIFDDVQAPNYTQHTSSMRGVYYP |
| | N-terminal | DEIFRSDTLYLTQDLFLPFYSNVTGFH |
| | without a | TINHTFGNPVIPFKDGIYFAATEKSNV |
| | signal | VRGWVFGSTMNNKSQSVIIINNSTNVV |
| | peptide | IRACNFELCDNPFFAVSKPMGTQTHTM |
| | | IFDNAFNCTFEYISDAFSLDVSEKSGN |
| | | FKHLREFVFKNKDGFLYVYKGYQPIDV |
| | | VRDLPSGFNTLKPIFKLPLGINITNFR |
| | | MLTAFSPAQDIWGTSAAAYFVGYLKPT |
| | | TFMLKYDENGTITDAVDCSQNIPLAEL |
| | | KCSVKSFEIDKGIYQTSNFRVVPSGDV |
| | | VRFPNITNLCPFGEVFNATKFPSVYAW |
| | | ERKKISNCVADYSVLYNSTFFSTFKCY |
| | | GVSATKLNDLCFSNVYADSFVVKGDDV |
| | | RQIAPGQTGVIADYNYKLPDDFMGCVL |
| | | AWNTRNIDATSTGNYNYKYRYLRHGKL |
| | | RPFERDISNVPFSPDGKPCTPPALNCY |
| | | WPLNDYGFYTTTGIGYQPYRVVVLSFE |
| | | LLNAPATVCGPKLSTDLIKNQCVNFNI |
| | | FNGLTGTGVLTPSSKRFQPFQQFGRDV |
| | | SDFTDSVRDPKTSEILDISPCAFGGVS |
| | | VITPGTNASSEVAVLYQDVNCTDVSTA |
| | | IHADQLTPAWRIYSTGNNVFQTQAGCL |
| | | IGAEHVDTSYECDIPIGAGICASYHTV |
| | | SLLRSTSQKSIVAYTMSLGADSSIAYS |
| | | NNTIAIPTNFSISITTEVMPVSMAKTS |
| | | VDCNMYICGDSTECANLLLQYGSFCTQ |
| | | LNRALSGIAAE |
| 57 | 272-537 | ITDAVDCSQNPLAELKCSVKSFEIDKG |
| | | IYQTSNFRVVPSGDVVRFNITNLCPFG |
| | | EVFNATKIFPSVYAWERKKISNCVADY |
| | | SVLYNSTFFSTFKCYGVSATKLNDLCF |
| | | SNVYADSFVVKGDDVRQIAPGQTGVIA |
| | | DYNYKLPDDFMGCVLAWNTRNIDATST |
| | | GNYNYKYRYLRHGKLRPFERDISNVPF |
| | | SPDGKPCTPPALNCYWPLNDYGFYTTT |
| | | GIGYQPYRVVVLSFELLNAPATVCGPK |
| | | LSTDLIKNQCVNFNFNGLTGTGV |
| 58 | 24-39 | DVQAPNYTQH TSSMRGC |
| | D24 peptide | |
| 59 | 540-555 | PSSKRFQPFQQFGRDC |
| | P540 peptide | |
| 60 | 1-16 | MFIFLLFLTLTSGSDL |
| | spike | |
| | signal | |
| | sequence | |
| 61 | 303-537 | SNFRVVPSGDVVRFPNITNLCPFGEVF |
| | containing the | NATKFPSVYAWERKKISNCVADYSVLY |
| | receptor | NSTFFSTFKCYGVSATKLNDLCFSNVY |
| | binding domain | ADSFVVKGDDVRQIAPGQTGVIADYNY |
| | | KLPDDFMGCVLAWNTRNIDATSTGNYN |
| | | YKYRYLRHGKLRPFERDISNVPFSPDG |
| | | KPCTPPALNCYWPLNDYGFYTTTGIGY |
| | | QPYRVVVLSFELLNAPATVCGPKLSTD |
| | | LIKNQCVNFNFNGLTGTGV |
| 62 | 319-517 | ITNLCPFGEVFNATKFPSVYAWERKKI |
| | containing | SNCVADYSVLYNSTFFSTFKCYGVSAT |
| | the receptor | KLNDLCFSNVYADSFVVKGDDVRQIAP |
| | binding domain | GQTGVIADYNYKLPDDFMGCVLAWNTR |
| | | NIDATSTGNYNYKYRYLRHGKLRPFER |
| | | DISNVPFSPDGKPCTPPALNCYWPLND |
| | | YGFYTTTGIGYQPYRVVVLSFELLNAP |
| | | ATVCGPKLST |
| 63 | 319-518 | ITNLCPFGEVFNATKFPSVYAWERKKI |
| | containing the | SNCVADYSVLYNSTFFSTFKCYGVSAT |
| | receptor | KLNDLCFSNVYADSFVVKGDDVRQIAP |
| | binding domain | GQTGVIADYNYKLPDDFMGCVLAWNTR |
| | | NIDATSTGNYNYKYRYLRHGKLRPFER |
| | | DISNVPFSPDGKPCTPPALNCYWPLND |
| | | YGFYTTTGIGYQPYRVVVLSFELLNAP |
| | | ATVCGPKLSTD |
| 66 | 317-517 | NLCPFGEVFNATKFPSVYAWERKKISN |
| | containing | CVADYSVLYNSTFFSTFKCYGVSATKL |
| | the receptor | NDLCFSNVYADSFVVKGDDVRQIAPGQ |
| | binding domain | TGVIADYNYKLPDDFMGCVLAWNTRNI |
| | | DATSTGNYNYKYRYLRHGKLRPFERDI |
| | | SNVPFSPDGKPCTPPALNCYWPLNDYG |
| | | FYTTTGIGYQPYRVVVLSFELLNAPAT |
| | | VCGPKLST |

### EXAMPLE 6

### Structure of the Spike Protein

To characterize the properties and function of the SARS-CoV S protein, nucleic acids encoding the full-length Tor2 isolate were cloned into expression vectors as described above. The Tor2 isolate is further described in Marra et al. The genome sequence of the SARS-associated coronavirus, Science 300:1399-1404 (2003). Clones generated included the full-length S protein (1255 residues), the ectodomain Se (residues 17-1189) having just the extracellular domain of the S protein with the putative transmembrane domain and cytoplasmic tail of the spike protein deleted, fragments containing the N-terminal 276 (SEQ ID NO:50), 537 (SEQ ID NO:52), and 756 (SEQ ID NO: 56) amino acid residues (S276, S537, and S756, respectively) including a putative 16-residue signal sequence or a mouse k chain leader sequence, and an internal fragment (S272-537) containing residues 272-537 (SEQ ID NO:57) (see FIG. 1B).

Amino acid residues 758-761 (RNTR) form part of the following general motif for cleavage by precursor convertases:
K/R-Xn-K/R
   where X is any amino acid residue and n=0, 2, 4 or 6.

The S1 subunit is approximately encompassed within the S756 fragment. This finding is in agreement with the size of the S1 subunit for murine coronaviruses, e.g., strain JHM where S1 is 769 residues, and for the human coronavirus OC43 (778 residues). See Gallagher &. Buchmeier, Coronavirus spike proteins in viral entry and pathogenesis, Virology 279: 371-374 (2001); Kunkel & Herrler, Structural and functional analysis of the surface protein of human coronavirus OC43, Virology 195 417: 195-202 (1993). However, for the human coronavirus 229E, S1 is considered to consist of a shorter 547 residue fragment that corresponds to S537. Bonavia et al., Identification of a receptor-binding domain of the spike glycoprotein of human coronavirus HCoV-229E, J. Virol. 77: 2530-2538 (2003).

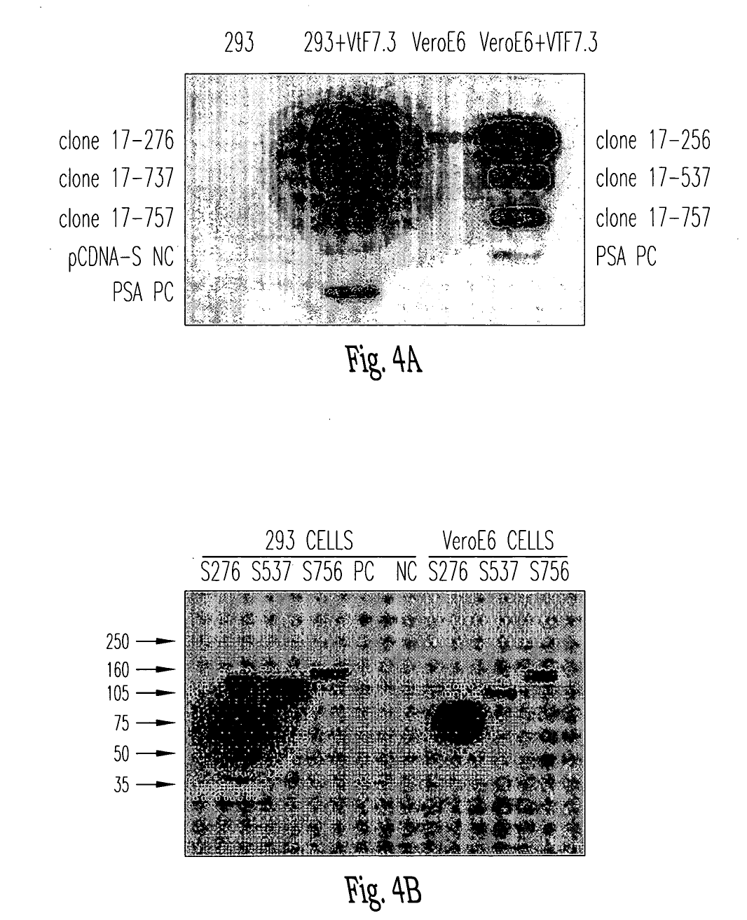

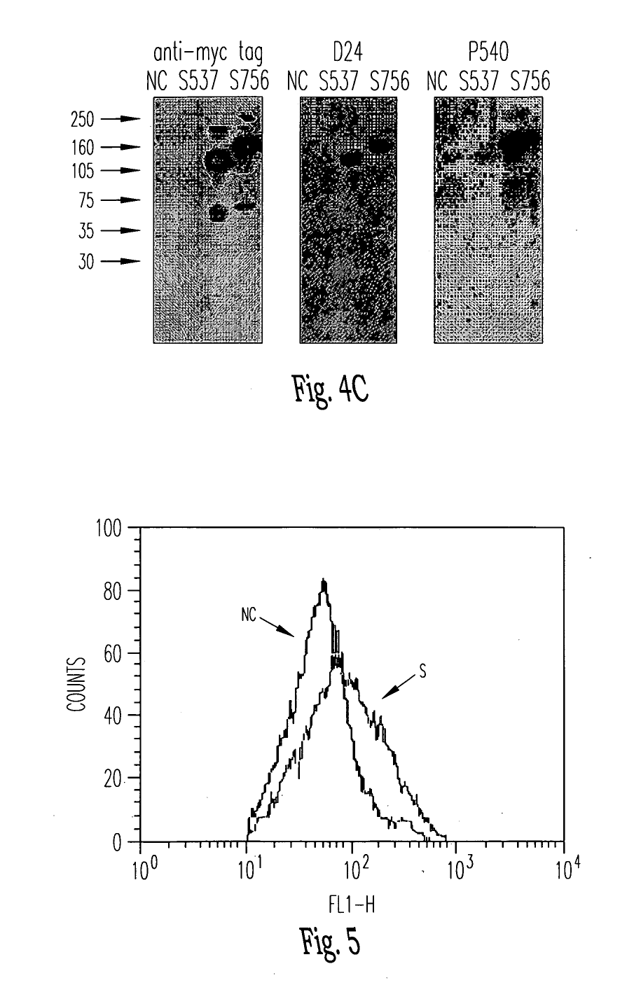

All S glycoprotein fragments and the full-length S glycoprotein ran on SDS-PAGE gels at positions significantly higher than their estimated molecular weights, indicating that these polypeptides are likely post-translationally modified. The S276 polypeptide had an apparent molecular weight of about 75 kDa, S537 had an apparent molecular weight of about 100-110 kDa, S756 had an apparent molecular weight of about 130-140 kDa, and Se and S had apparent molecular weights of about 200 kDa or higher (FIGS. 4 and 6). The bands corresponding to these polypeptide were broad even when observed at low exposure (FIG. 6; some data not shown). These data indicate significant glycosylation as observed for the S glycoprotein and fragments thereof. Based on approximate estimations of molecular weight it appears that the S2 subunit is not as heavily glycosylated as S756 (constituting the S1 subunit). Notably, S276 is heavily glycosylated if one assumes that only glycosylation contributes to the increased molecular mass.

Most of the SARS-CoV S glycoprotein obtained from cell culture supernatants was not cleaved, although weak bands due to smaller proteins were observed on SDS-PAGE gels. One of these weak bands runs at the same position as S756, suggesting the possibility of inefficient cleavage (FIGS. 4 and 6). Random digestion by proteases may occur and further studies are needed to determine if the S glycoprotein cleavage is necessary for its function.

### EXAMPLE 7

### Expression of Peptide Fragments in Escherichia coli

A nucleic acid segment encoding a SEQ ID NO: 51 peptide fragment containing amino acid residues 17-446 of SEQ ID NO: 1 was cloned into the pRSET vector (Invitrogen, San Diego, Calif.) to create the plasmid pRSET-S(17-446). E. coli BL21DE3 cells were transformed with pRSET-S(17-446) and then induced with IPTG. The results of the induction are shown in FIG. 2.

### EXAMPLE 8

### Use of the T7 Promoter to Drive Expression of a Cloned Peptide Fragment of the Invention

Human 293 cells or Monkey Vero E6 cells were grown to a density of 1.2×10⁶ cells/T25 flask (60 mm dish) in 5 ml of DMEM+10% FBS medium the day prior to transfection. The cells were then transfected, using the Polyfect (Qiagen) transfection kit according to the manufacturer's protocol, with pSecTag2B constructs (6 ug each) containing inserts coding for the various peptide fragments of the spike protein. These constructs were prepared as described above.

After 4 hour of transfection, a VTF7.3 vaccinia virus carrying a T7 polymerase was used to infect the transfected cells at a MOI (multiplicity of infection) of 20 (Fuerst et al., Proc. Natl. Acad. Sci., 93:11371 (1986)). This procedure provided for the use of the T7 promoter in the pSecTag2B vector instead of the CMV promoter, which is much weaker (Nussbaum et al., J. Virol., 68:5411 (1994)). After three hours of infection, 1.5 ml of fresh medium was added to the cells and then the cells were transferred to a 31° C. incubator. The cells were incubated for an additional 24 hours, after which the culture medium was collected.

No measurable cytopathicity was observed in cells transfected with any of the S nucleic acid constructs (data not shown), indicating that the full-length and soluble fragments of the S glycoprotein may not have significant cytotoxic effects. However, at higher levels of expression such effects are possible and formation of syncytia as described below may lead to cell death.

### EXAMPLE 9

### Spike-Specific Antibodies

New Zealand rabbits were immunized with 0.1 mg of various peptides selected by a computer program for their immunogenicity. Serum from the immunized rabbits was tested in ELISA and Western blot for reactivity. Sera from rabbits immunized with two peptides exhibited the highest and specific activity against the spike glycoprotein and were selected for further study. Antibodies denoted D24 and P540 were elicited by the peptides DVQAPNYTQH TSSMRGC (SEQ ID NO:58) and PSSKRFQPFQQFGRDC (SEQ ID NO:59), respectively. Another anti-SARS-CoV S glycoprotein polyclonal antibody IMG-542, which recognizes amino acid 288-303 of the S glycoprotein, was purchased from IMGENEX (San Diego, Calif.).

### EXAMPLE 10

### Immunoprecipitation and Purification of Spike Polypeptides

Soluble spike polypeptides fragments were obtained from the Vero E6 or 293 cell culture medium. However, the full-length spike glycoprotein was detected only in the cell lysate.

Medium from cells transfected with nucleic acids encoding various soluble S fragments was collected and subjected to centrifugation at 1000 g for 10 min to remove cellular debris. The cleared medium was incubated with either Ni-NTA agarose beads (Qiagen, Valencia, Calif.) or an immunoprecipitating antibody plus glycoprotein G-Sepharose beads (Sigma, St. Louis, Mo.) for 2 h at 4° C. The beads were then mixed with an equal volume of SDS gel sample buffer, boiled for 3 min, and subjected to gel analysis. For full-length S glycoprotein, cells were lysed first in PBS supplemented with 1% NP-40 and 0.5 mM PMSF for 1 h at 4° C., and centrifuged at 14,000 rpm in a table-top Eppendorf centrifuge for 20 min. The cleared lysate was either immunoprecipitated first or used directly in Western blotting.

### EXAMPLE 11

### Western Blotting and Slot Blots

Cells expressing the S glycoprotein were lysed first with a PBS-based NP40 lysis buffer as described above, and the debris was cleared by centrifugation. For soluble S fragments the medium was collected and cleared as described above. For slot blots, the cleared lysate or medium from supernatant was used directly to blot the nitrocellulose membrane following the protocol suggested by the manufacturer (Bio-Rad, Hercules, Calif.) and the membrane was subjected to antibody detection as in conventional Western blotting. For Western blotting, a monoclonal anti-c-Myc epitope antibody (Invitrogen, Carlsbad, Calif.) or anti-spike protein rabbit polyclonal antibodies obtained by immunization of rabbits with spike peptides were diluted in TBST buffer. Antibodies were incubated with the membrane for 2 h, washed and then the membrane was incubated with a secondary antibody conjugated with HRP for 1 h, washed four times (each time for 15 min), and then developed using the ECL reagent (Pierce, Rockford, Ill.).

### EXAMPLE 12

### Cell-Binding Assay and ELISA

Medium containing soluble S fragments was collected and cleared by centrifugation. Vero E6 or other cells (5×10⁶) were incubated with 0.5 ml of cleared medium containing soluble S fragments and 2 μg of anti-c-Myc epitope antibody conjugated with HRP at 4° C. for 2 h. Cells were then washed three times with ice-cold PBS and collected by centrifugation. The cell pellets were incubated with ABTS substrate from Roche (Indianapolis, Ind.) at RT for 10 min, the substrate was cleared by centrifugation, and the optical density at 405 nm was measured. The result of the slot blot analysis is presented in FIG. 4 and discussed in further detail below.

For ELISA, purified ACE2 (R&D, Minneapolis, Minn.) was adsorbed onto Maxisorp ELISA plates in pH 9.6 buffer at a concentration of 100 ng per well. Medium 154 (150 μl) containing various soluble S fragments and 0.6 μg of anti-c-155 Myc epitope antibodies conjugated with HRP were incubated in each well at 37° C. for 2 h. Wells were washed and 60 μl of ABTS substrate was added to each well. The optical density (OD₄₀₅) was measured 20 min later.

### EXAMPLE 13

### Fluorescent Dye Redistribution Cell Fusion Assay

HeLa or 293T cells, transfected with plasmids encoding the S glycoprotein, were loaded with Calcein AM (Molecular Probes), which is converted within the cells to calcein green. The cells were incubated in medium containing 1 μg/ml Calcein AM for 1 h at 37° C. and 5% CO₂, and then washed and re-suspended in fresh medium. Plated target cells, Vero E6, were stained with CMAC (Molecular Probes) by incubation in 1 μg/ml CMAC in medium for 30 min at 37° C. and 5% CO₂. The cells were then washed twice with medium, incubated for 20 min in fresh medium, washed again, and then covered with 0.5 ml medium per well. The S-expressing cells, loaded with calcein, were added to the target cells and incubated for 1, 2, or 4 h at 37° C. and 5% CO₂. Fusion was measured as the ratio between the cells that have double staining and the total number of target cells in contact with an S glycoprotein-expressing cell. Microphotographs were taken using the MethaMorph 4.0 software from Universal Imaging.

### EXAMPLE 14

### β-Galactosidase Reporter Gene-Based Cell-Cell Fusion Assay

293T cells (1.5×10⁶) were plated in T25 flasks. The next day, these cells were separately transfected with pCDNA3-S, pSectag2B-S, pCDNA3-ACE2, and pCDNA3-ACE2-Ecto using the Polyfect transfection kit (Qiagen, Valencia, Calif.) following the manufacturer's suggested protocol. Four hours after transfection, cells transfected with S constructs were infected with T7 polymerase-expressing vaccinia virus VTF7.3 and cells transfected with ACE-2 constructs were infected with β-gal encoding vaccinia virus (VCB21R). Two hours after infection, cells were incubated with fresh medium and transferred to 31° C. for overnight incubation. The next day S glycoprotein-expressing cells and ACE-2-expressing cells were mixed in a 1:1 ratio and incubated at 37° C. Three hours later, cells were lysed by adding NP-40 to a final concentration of 0.5%. Cell lysate (50 μl) was mixed with equal volume of CPRG substrate and OD₅₉₅ was measured 1 hr later.

### EXAMPLE 15

### Expression of Spike Polypeptides in Mammalian Cells

For certain experiments, all proteins except the full-length S glycoprotein were tagged with a c-Myc epitope and a histidine tag. These proteins were expressed in 293 and Vero E6 cells after transfection with the corresponding plasmids followed by infection with vaccinia virus-expressing T7 polymerase.

The tagged proteins were detected by using an anti-c-Myc monoclonal antibody (FIG. 4). As shown in FIG. 4, the T7 promoter was a highly efficient promoter for expression of the S glycoprotein. In these experiments, the T7 promoter gave rise to higher levels of expression than the CMV promoter, which under most circumstances is a strong promoter (FIG. 4A). As shown in FIG. 4A, the S fragments were soluble and their concentration in the culture supernatants was inversely proportional to their size.

### EXAMPLE 16

### Anti-Spike Antibodies

To be able to detect unlabeled proteins, validate the data obtained by the anti-c-Myc antibody, and localize possible antigenic sites rabbit polyclonal antibodies were developed. Two of these antibodies, D24 and P540, were raised against peptides starting at residues 24 and 540, respectively. The D24 and P540 antibody preparations specifically recognized certain soluble fragments (FIG. 4C). As expected, D24 recognized all fragments; P540 recognized S756, Se, and S but not the smaller fragments (FIG. 4C; some data not shown). The D24 antibody preparation was relatively weak. However, the P540 preparation was very sensitive even at dilution 1:10,000 and was used extensively in the experiments described herein.

The P540 antibody preparation was used to detect whether the S glycoprotein was expressed intracellularly, extracellularly or on the cell surface. As shown in FIG. 5, the full-length S glycoprotein was expressed at the cell surface, although at low levels, as measured by flow cytometry.

### EXAMPLE 17

### Spike Protein Mediates Cell Fusion

The full-length S glycoprotein mediates fusion at neutral pH with cells expressing receptor molecules. Cell-cell fusion assays were performed to confirm that the full-length recombinant S glycoprotein was functional, and to ascertain whether the S protein requires other viral proteins and/or low pH for its fusion activity.

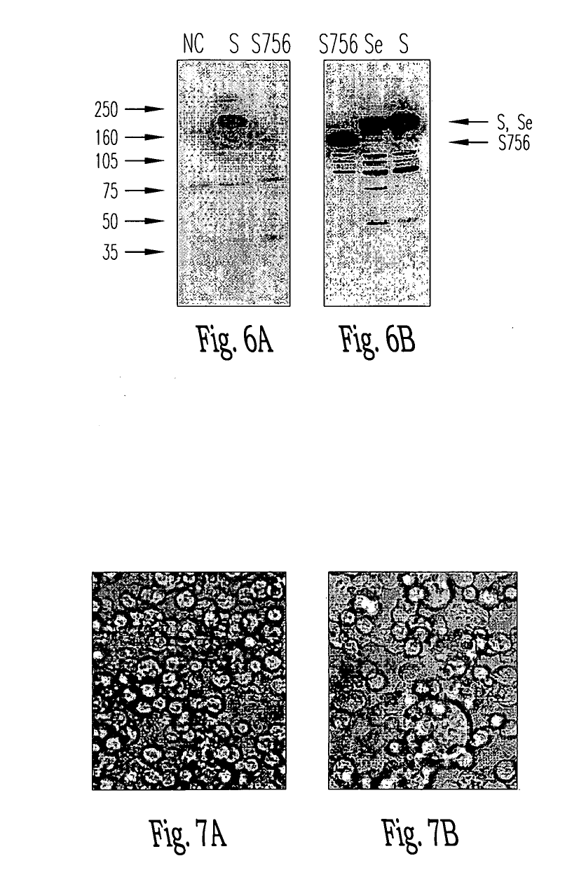

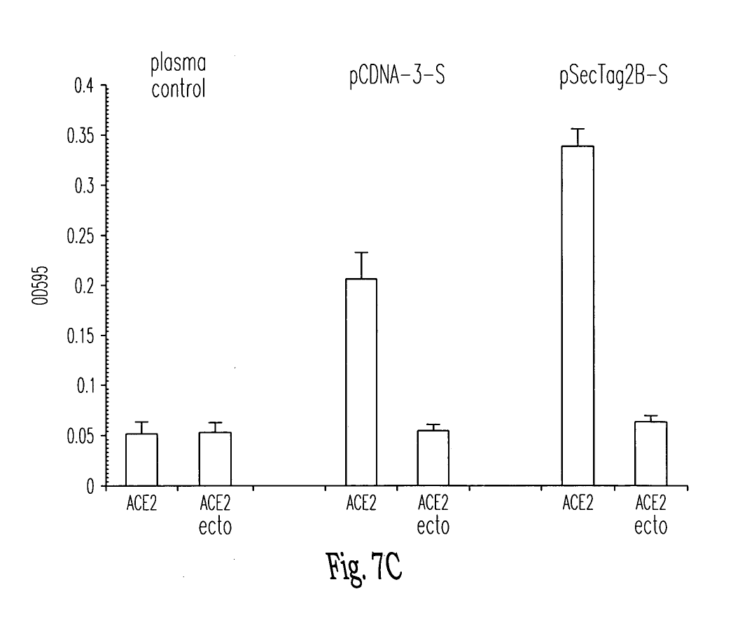

Expression of the full-length S glycoprotein with both vectors pCDNA3-S and pSectag2B-S, supported fusion with ACE2 expressing cells efficiently, as evidenced by formation of syncytia of various sizes and by β-gal reporter gene-based assay (FIG. 7). Interestingly, the pSectag2B-S construct in which the S glycoprotein leader peptide was replaced by a mouse k chain leader sequence induced faster formation of syncytia. Moreover, the syncytia formed were larger and more numerous than those induced by pCDNA3-S, which encodes the native S glycoprotein (data not shown). The extent of fusion mediated by S expressed from pSectag2B-S was also higher than from pCDNA3-S as measured by a reporter gene-based assay (FIG. 7B). These data indicate that the natural S glycoprotein may not be efficiently transported to the cell surface. These studies also suggest that the β-gal assay described here can serve as a quick and quantitative method to identify inhibitors of SAR-CoV entry into cells, as well as a tool to study SARS-CoV entry mechanism.

Notably, fusion of Vero E6 cells was not detected using the β-gal assay or the syncytium formation assay when the cells were not transfected with plasmids encoding ACE2 and the cells expressed only native concentrations of the receptor. To explore the possibility that this was due to low sensitivity of these two assays, another assay was used. This new assay was based on fluorescent dye redistribution that is able to detect fusion of single cells. Even with this fluorescent-based assay statistically significant differences between cells transfected with plasmids encoding the full-length S glycoprotein and various negative controls were not detected. Some of the negative controls included transfection with plasmids encoding soluble S fragments at different pH (data not shown). Significant cell-cell fusion was only detected when the cells were transfected with plasmids encoding ACE2, suggesting that the higher levels of receptor expression achieved by expression of recombinant ACE2 could be important for cell-cell fusion. Overall, these results suggest that recombinant S glycoprotein can mediate cell fusion, that fusion can occur at neutral pH, and that its efficiency is dependent on the concentration of the receptor molecules.

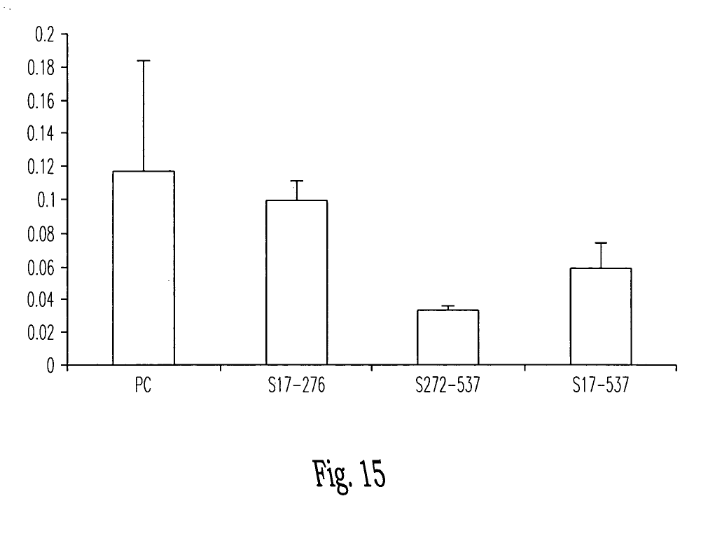

Moreover, soluble fragments of the S glycoprotein inhibit S-mediated cell fusion. As shown in FIG. 15, addition of S fragments S272-537 and S17-537, which have the receptor binding domain as described below, inhibit S-mediated cell fusion. In this assay, the S272-537 (SEQ ID NO:57) fragment, exhibited the most inhibition. The S17-276 fragment that does not have the receptor binding domain exhibited little or no inhibition of S-mediated cell fusion. These data indicate that S polypeptide fragments that have the receptor binding domain could inhibit SARS-CoV fusion with animal cells, thereby inhibiting or preventing SARS-CoV infection.

Hence, blocking, modulating or inhibiting the activity of the spike protein receptor binding domain, with an anti-RBD antibody, S polypeptide, S peptide or aptamer may be an effective preventive or treatment for SARS-CoV infection.

### EXAMPLE 18

### Identification of Spike Protein Receptor-Binding Domain

This Example illustrates that the Spike protein receptor-binding domain is localized within residues 272 to 537 (SEQ ID NO:57), and likely within residues 303-537 (SEQ ID NO:61). Later experiments have shown that a fragment containing residues 319-517 (SEQ ID NO:62) also has receptor binding activity.

An assay based on the binding of various soluble fragments to receptor expressing Vero E6 cells was developed to localize the receptor-binding domain (RBD) of the S glycoprotein. This assay involved measurement of fluorescence associated with binding of antibodies directed against the S polypeptides to Vero E6 cells and was developed prior to the identification of the SARS-CoV receptor. Vero E6 cells that are susceptible to SARS-CoV infection were incubated with full-length S polypeptide and various soluble S fragments. Several cell lines that are not susceptide to SARS-CoV infection were similarly incubated with full-length S polypeptides and soluble fragments thereof.

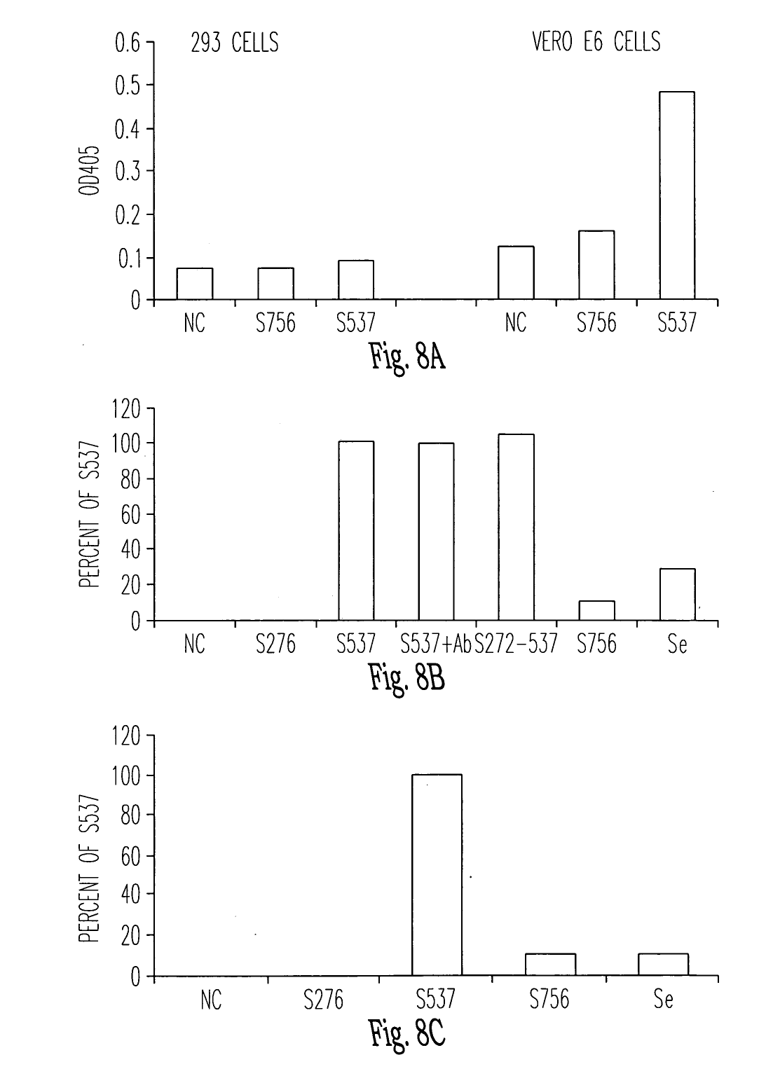

As shown in FIGS. 8A and 8B, all fragments S fragments bound to Vero E6 cells except the smallest one S fragment (S276). No such binding was detected when several cell lines that are not susceptide to SARS-CoV infection were incubated with full-length S polypeptides and soluble fragments thereof. Binding to Vero E6 cells was proportional to the expression levels of the fragments and was approximately inversely proportional to the sizes of the fragments. These findings suggested that the RBD is localized between residues 272 and 537.

To further localize the RBD, an antibody (IMG 542) was used that was generated using a peptide containing residues 288-303. This antibody did not inhibit binding of the S537 fragment to Vero E6 cells although it did bind to the S537 fragment (FIG. 8B; some data not shown), suggesting that the RBD is localized between residues 303 and 537. Because of the relatively large antibody size and the possibility for steric hindrance, it is likely that the RBD is downstream of residue 303. Recently, the RBD of the HCoV-229E was localized to a fragment containing amino acid residues 407-547. Ksiazek et al. A novel coronavirus associated with severe acute respiratory syndrome, N. Engl. J. Med. 348: 1953-1966 (2003); Rota et al. Characterization of a novel coronavirus associated with severe acute respiratory syndrome, Science 300: 1394-1399 (2003). In contrast, the RBD for murine hepatitis virus was mapped to the N-terminal 330 amino acids.

It remains to be seen whether there is structural similarity between the RBD-containing fragments of the SARS-CoV S1 glycoprotein (e.g., S272-537) and the HCoV-229E or hepatitis virus RBD, and whether such similarity is related to the use of the same host for replication. These two viruses use different receptors. The straightforward cell-binding approach described here could also be helpful for identification of other virus receptors.

Recently, workers have reported the identification of ACE2 as a functional receptor for the SARS-CoV. Li et al. Angiotensin-converting enzyme 2 is a functional receptor for the SARS coronavirus, Nature 426: 450-54 (2003). The identification of ACE2 as receptor permitted further validation that the results provided above are correct. As shown in FIG. 8C, when purified ACE2 is used in an ELISA to test for binding, the same binding pattern was observed as for the cell-binding assay. This was true for all of the S fragments tested (FIG. 8C).

The results provided herein not only offer new tools to study entry of the SARS virus into cells, confirm that ACE2 is a receptor for the SARS-CoV S1 glycoprotein and localize the RBD but also facilitate development of novel vaccine immunogens and therapeutics for prevention and treatment of SARS.

### EXAMPLE 19

### N-Terminal and C-Terminal Oligomerization of the S Glycoprotein

This Example illustrates that the extreme N-terminal fragment of the S glycoprotein, upstream from the RBD, may play a role in fusion, and the S ectodomain forms trimers that could mediate fusion through six-helix bundle intermediates.

Materials and Methods

Antibodies and plasmids. The rabbit anti-S serum used in Western and FACS analyses, P540 was developed by the inventors as described above. See also, Xiao et al. Biochem. Biophys. Res. Comm. 312: 1159-65 (2003). The anti-Myc epitope antibody was purchased from Invitrogen (Carlsbad, Calif.). The anti-ACE2 goat polyclonal antibody was purchased from R&D system (Minneapolis, Minn.) and used for detection by Western blotting.

Site directed mutagenesis was used to create the consensus cleavage sites corresponding to that of the HIV-1 envelope glycoprotein (Env) and some coronaviruses within the full length SARS-CoV S glycoprotein gene in pCDNA3. The QuickChange Kit from Stratagene (La Jolla, Calif.) was employed using the protocol provided by manufacturer. For expression of various N terminal S fragments, the corresponding gene fragments were amplified by PCR and cloned into the pSecTag2 expression vector (Invitrogen, Carlsbad). The plasmid pCDNA3-ACE2-ecto, which expresses the ACE2 soluble ectodomain tagged with C9 peptide was kindly provided by Michael Farzan (Harvard University, Boston Mass.).

Protein expression and purification. Various N terminal fragments of the S glycoprotein were sub-cloned in pSecTag2 expression vector and transfected into 293T cells followed by infection with VTF7.3 as described in Xiao et al. Biochem. Biophys. Res. Comm. 312: 1159-65 (2003). The protein expressed and secreted into the medium was purified using the HiTrap Ni⁺⁺-Chelating column (Pharmacia) under native conditions. The purified protein was dialyzed against PBS buffer and stored for further analysis.

S glycoprotein dimerization and its interaction with ACE2 examined by co-immunoprecipitation. For S fragment dimerization, different S glycoprotein constructs, alone or in combination, were transfected to 293T cells as described in Xiao et al. Biochem. Biophys. Res. Comm. 312: 1159-65 (2003). Medium containing S fragments was subjected to immunoprecipitation with rabbit anti-S polyclonal antiserum P540. For some co-immunoprecipitation experiments, DTT was added to create reducing condition to eliminate inter-molecule interactions through disulfide bonds. Immunoprecipitated S fragments were detected by Western using an anti-Myc epitope monoclonal antibodies. Soluble ACE2-C9 was expressed similarly. ACE2-C9 secreted into the medium was used directly for incubation with various S fragments for 2 hours at 4° C. Afterwards, ACE2 was immunoprecipitated by incubating with 1D4 anti-C9 monoclonal antibody and protein G-Sepharose beads at 4° C. for one hour. The beads were washed four times with PBS, suspended in SDS-PAGE sample buffer, boiled for 3 min and subjected to gel separation. The presence of either ACE2 or S in the sample was examined by Western as described in Xiao et al. Biochem. Biophys. Res. Comm. 312: 1159-65 (2003).

Flow cytometry. Cells transfected with full length S glycoprotein or S glycoprotein with different N terminal deletions and infected with VTF7.3 were incubated with the P540 rabbit anti-S polyclonal antibody and goat anti-rabbit antibody conjugated with FITC in PBS containing 1% BSA at 4° C. for two hours. Cells were then washed four times in ice cold PBS and analyzed with FacsCalibur (Becton Dickinson, San Jose, Calif.).

Gel filtration analysis of S fragments. After being purified on Ni-chelating column and buffer-exchanged to PBS, S fragment samples were loaded onto a Superose 12 10/300 GL column (Pharmacia, Uppsala, Sweden) that had been pre-equilibrated with PBS. The proteins were eluted with PBS at 0.5 ml/min, and 0.5 ml fractions were collected. The Superose 12 column was calibrated with protein molecular mass standard of 669, 440, 232, 158, 67, 44 and 25 kD. A 10 μl aliquot was taken from each fraction for Western blot analysis.

Crosslinking. Purified S537 fragment was diluted to a concentration of 0.2 μg/ml in PBS. BS³ (Pierce, Rockford, Ill.) was added to the S537 solution to a final concentration of 1 mg/ml and incubated on ice for 1 min. The samples were then mixed with an equal volume of 4×SDS-PAGE loading buffer and analyzed by Western blot.

Cell fusion β-gal reporter gene assay. Cells transfected with pSecTag2B-S or pCDNA3-ACE2 and infected with VTF7.3 and VCB21R respectively were collected by trypsin digestion and washed once with PBS. Cells were then suspended in regular DMEM medium at pH 7.4 and mixed. Cells were lysed after four hours of incubation and β-gal activity was measured using CPRG as the substrate (Roche) as described in Xiao et al. Biochem. Biophys. Res. Comm. 312: 1159-65 (2003).

ELISA. Two ELISA assays were used. In the sandwich ELISA the plate was coated with an anti-His tag antibody, then the S fragment were added and detected with an anti-c-Myc epitope antibody. This assay was used for detection of the S fragments. In the second ELISA assay the C9 tagged receptor ACE2 was coated on the plates through an anti-C9 antibody (ID4) and the S fragments were added and after washing detected with an anti-c-Myc epitope antibody. In all experiments the incubations with the c-Myc epitope antibody were for 2 hours at room temperature. The optical density (OD) was measured and normalized to the highest value.

Results

The N-terminal fragment upstream of the RBD of the S glycoprotein forms a dimer. It has been previously shown for another coronavirus (MHV) that soluble S1 (similar to SU) fragments form dimers, that the extreme N-terminal 330 amino acid residue region that contains the receptor binding domain participates in the dimerization, and that only dimers bind the receptor CEACAM. See Lewicki & Gallagher, J. Biol. Chem. 277:19727-34 (2002). However, the inventors and others have localized the SARS-CoV receptor binding domain downstream from the extreme N-terminus. Xiao et al. Biochem. Biophys. Res. Comm. 312: 1159-65 (2003); Wong et al. J. Biol. Chem. 279: 3197-3201 (2004); Babcock et al. J. Virol. 78: 4552-4560 (2004).

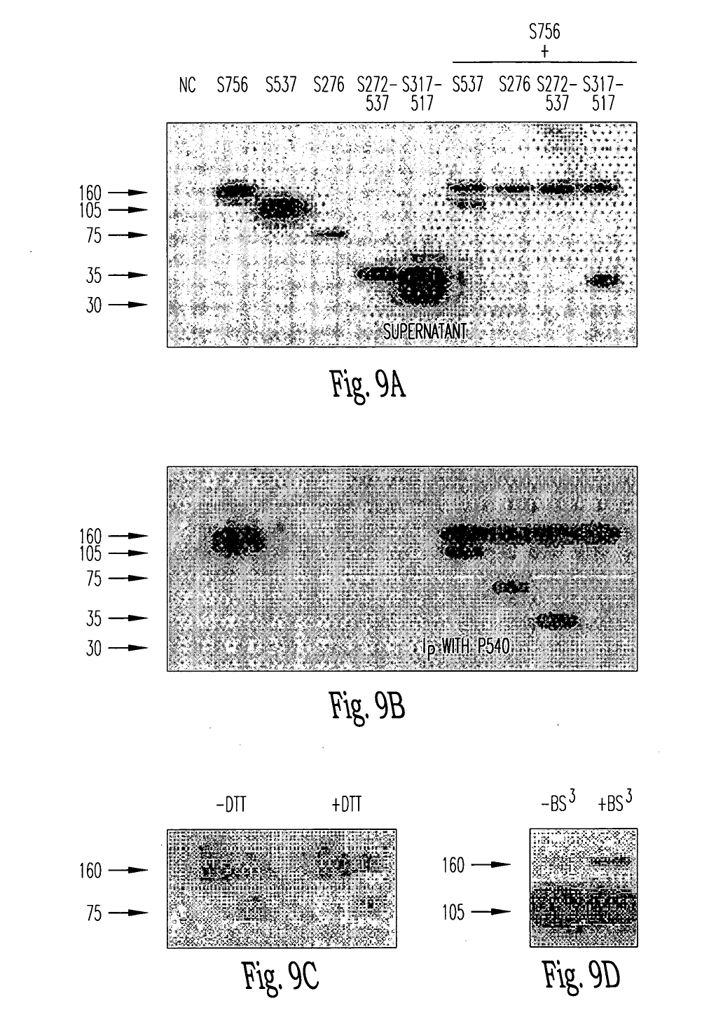

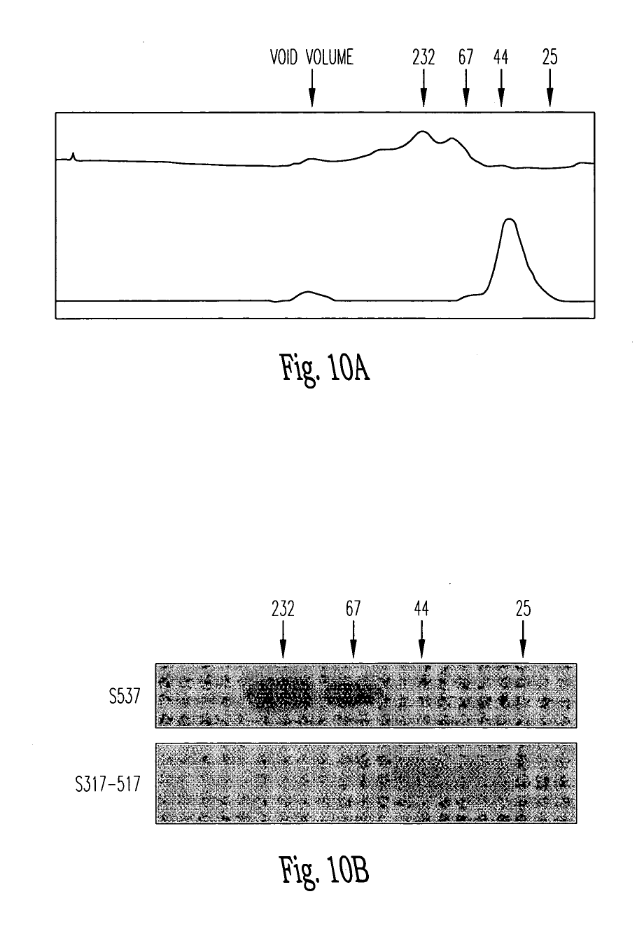

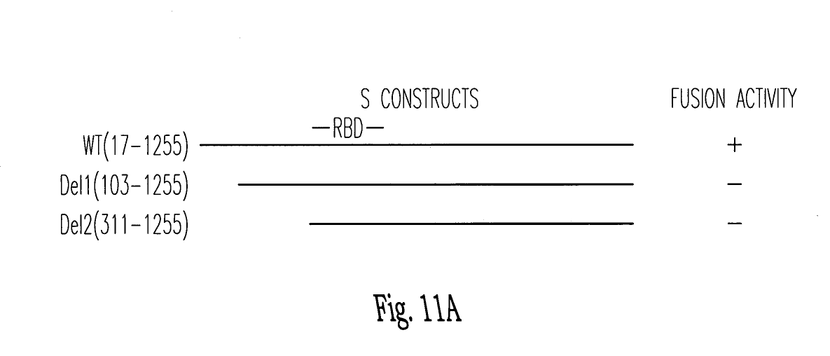

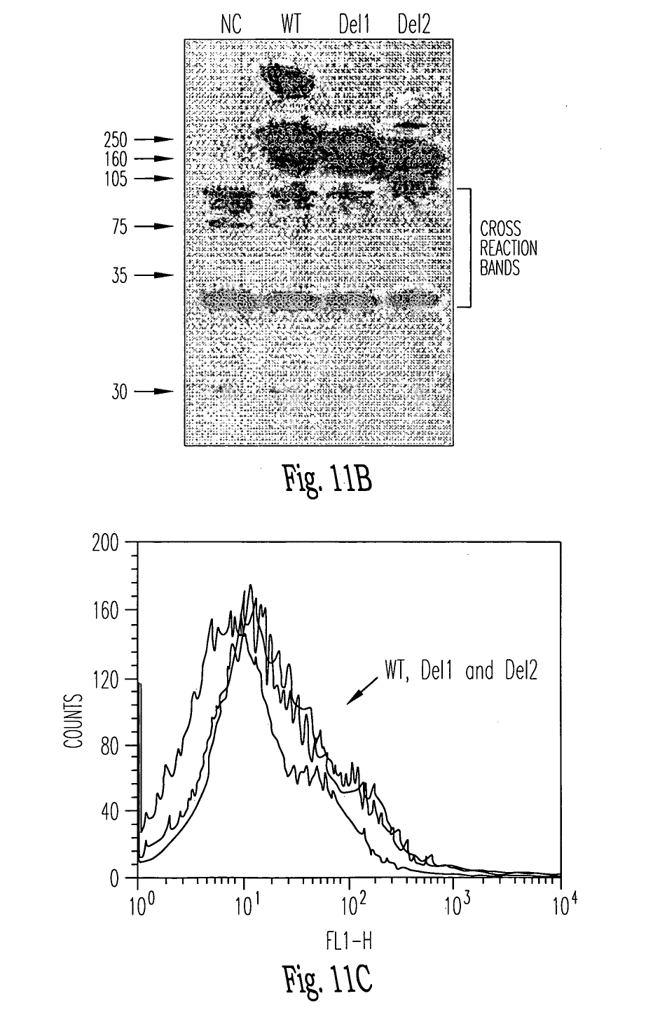

To address the possibility of oligomerization by receptor binding domain-containing fragments and to assess their function in mediating membrane fusion, several S fragments were tested for oligomerization. These S fragments included the extreme N-terminal fragment (residues 17 through 276 denoted as S276, SEQ ID NO:50) that does not bind the receptor ACE2, several S fragments (S756, S537, S272-537) that bind ACE2, as well as a fragment including residues 319 through 517 (denoted as S319-517, SEQ ID NO:62) that retains receptor binding activity. These fragments were selected in part because they fold independently and are secreted in the cell culture supernatant, although the efficiency of their expression varied significantly (FIG. 9A, left) and their concentration was decreased when co-expressed with S756 (FIG. 9A, right).

To find whether any of these fragments oligomerizes with the largest one (S756) that includes the equivalent of the receptor-binding subunit of the envelope glycoproteins (SU in general and S1 for coronaviruses) the polypeptide fragments were coexpressed, and then the mixtures in the cell culture supernatants were immunoprecipitated with the antibody P540. As described in previous Examples, this rabbit polyclonal antibody preparation was developed against a peptide containing residues 540-555 (SEQ ID NO:59) of the S glycoprotein. The P450 antibody binds the S756 polypeptide but not the other fragments (FIG. 9B, left). All N-terminal fragments except the smallest fragment (S319-517) containing the receptor binding domain were coimmunoprecipated with S756 by P540 (FIG. 9B, right). To rule out the possibility of nonspecific disulfide bond formation that may lead to coimmunoprecipitation, DTT was included in one of the coimmunoprecipitation experiments. DTT had no effect on either immunoprecipitation or coimmunoprecipitation of secreted S756 (left lanes) or S756+S276 (right lanes) (FIG. 9C, left panel).

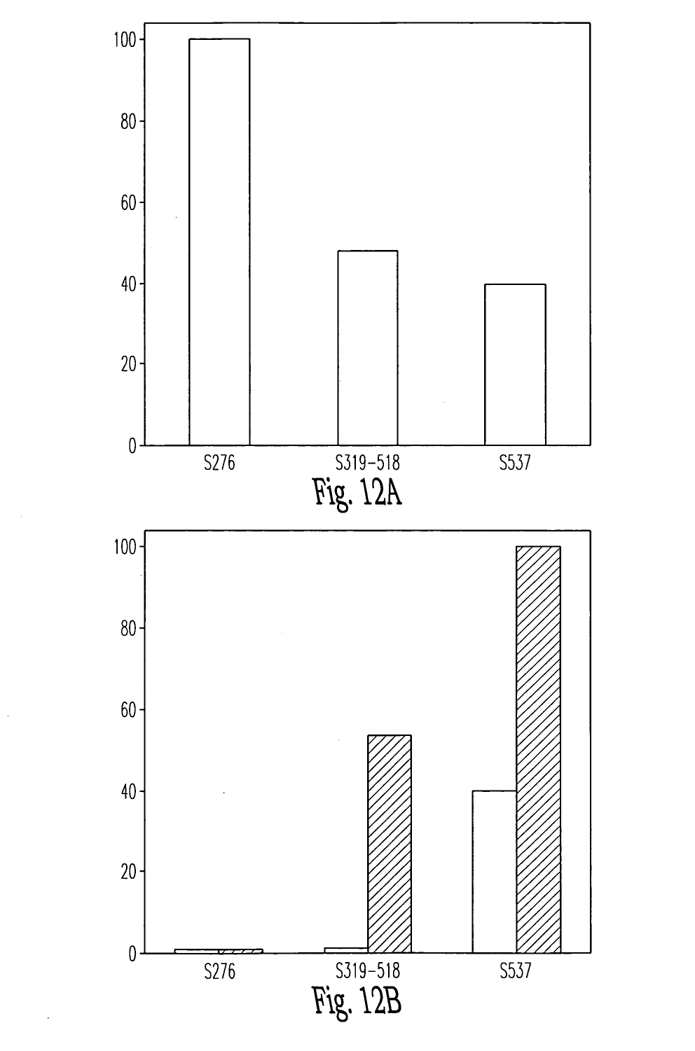

To find the size of the oligomers, one of the fragments (S537) was cross-linked with BS³. The right panel of FIG. 9C shows the appearance of a new band with a molecular weight corresponding to a dimer but not of higher order oligomers. To exclude the possibility of artifacts due to cross-linking and further to confirm the formation of dimers, the S537 fragment was also analyzed by gel filtration. Two gel filtration elution peaks were observed: one due to species of molecular weight of about 230 kDa and the other one of about 110 kDa (FIG. 10A, upper panel) corresponding to a dimer-sized oligomer and a monomer, respectively. In contrast, the smallest fragment containing the receptor binding domain (S319-517) was eluted only as a monomer at about 35 kDa molecular weight (FIG. 2A, lower panel). Overall, these results suggest that soluble SU is a dimer and that the dimerization domain is within the extreme N-terminal region upstream from residue 317 and the receptor binding domain.

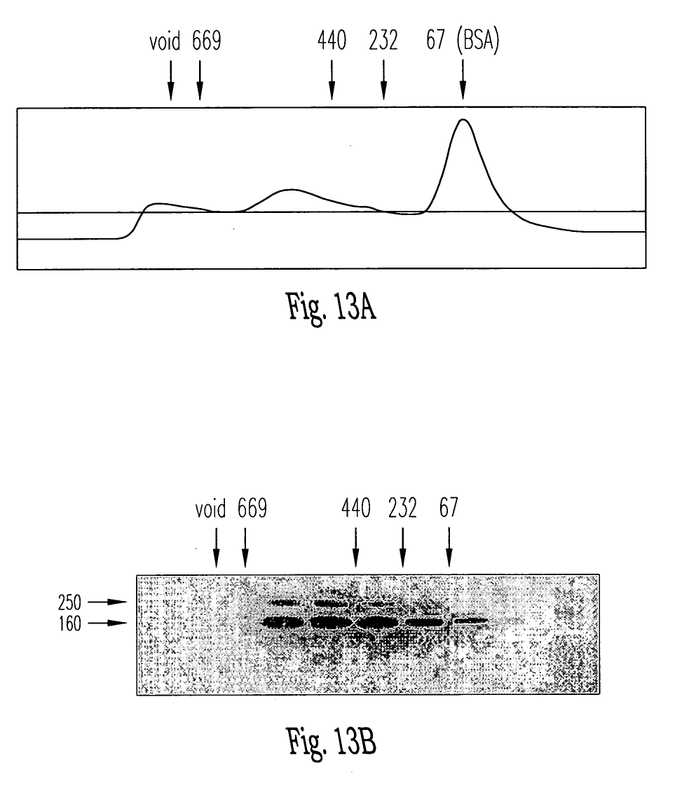

The dimeric N terminal region is required for S mediated cell-cell fusion. Because the putative dimerization domain is upstream from the receptor binding domain within S1 and the fusion machinery is in S2, one might hypothesize that dimerization may not be required for mediation of fusion. To test this hypothesis, two deletion mutants of the full-length S glycoprotein were generated. The N-terminal 103 residues were deleted from one fragments and the N-terminal 311 residues were deleted from another (FIG. 9A), thereby eliminating the presumed dimerization domain. Both mutants did not exhibit any fusion activity compared to the wild type full-length S glycoprotein, which did (FIG. 9A). To test whether a differential level of expression could account for the lack of observable fusogenic activity, the surface and overall levels of expression were measured by flow cytometry and Western blotting. The data from both assays suggested that the level of expression of the two deletion mutants is undistinguishable from that of the wild type (FIGS. 11B and C). These results suggest that the extreme N-terminus is required for fusion by a mechanism that may or may not involve dimerization.

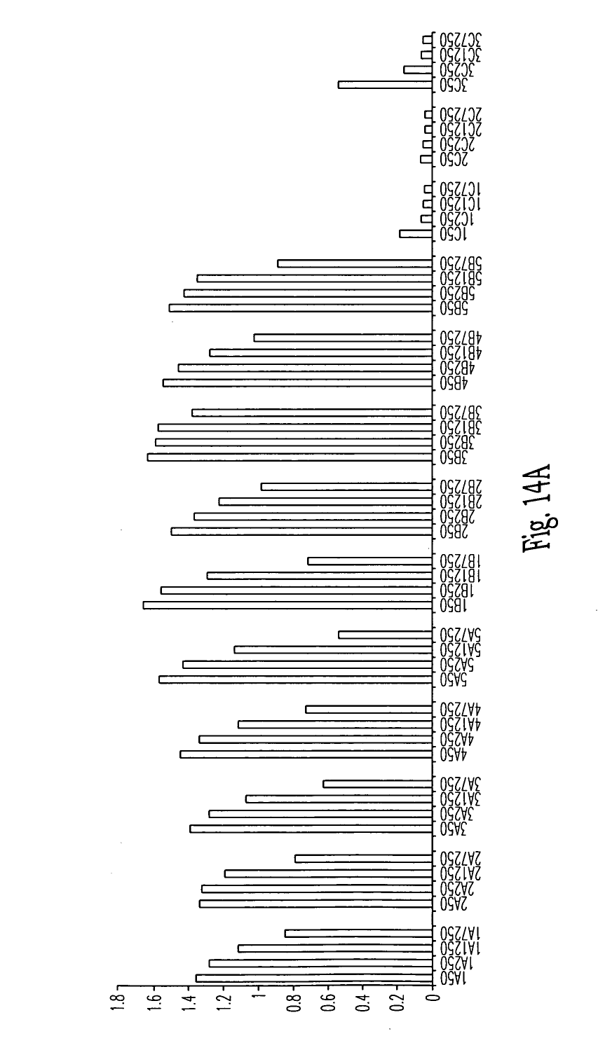

Dimeric S1 binds ACE2 much more efficiently than monomeric fragments containing the Receptor Binging Domain. Previous work with another coronavirus (MHV) suggested that only dimeric S1 binds its receptor CEACAM. Lewicki & Gallagher, J. Biol. Chem. 277:19727-734 (2002). Experiments were conducted on SARS-CoV fragments to understand how the dimeric state of the S1 may affect fusion. In particular, binding of S1 fragments in monovalent and bivalent form to ACE2 was observed by using the anti-c-Myc epitope antibody for conversion of monovalent S1 fragments into bivalent ones. One of these S1 fragments (S319-517, SEQ ID NO:62) did not bind to any measurable degree to surface-immobilized ACE2 unless bound by an anti-c-Myc epitope antibody, which converted it into a bivalent molecule in solution before and during incubation with the receptor (FIG. 12). In contrast, S537 bound to ACE2 without the antibody although the antibody presence increased its binding (FIG. 12). These results suggest that a dimeric state of S1 could contribute to an increased overall affinity that may enhance fusion efficiency.

The soluble S ectodomain is a trimer. Viral envelope glycoproteins of class I fusion proteins such as hemagglutinin (HA) of influenza are trimeric through the transmembrane domain. Because the SARS-CoV S glycoprotein was recently found to be class I fusion protein, the S2 subunit may facilitate trimerization of the whole S glycoprotein. However, a dimeric S1 with a trimeric S2 could lead to higher order oligomers whose formation depends on the availability of the dimerization binding site in the native S glycoprotein. To test this possibility the size of the soluble S ectodomains (Se) was approximated by gel filtration, where the transmembrane domain and the cytoplasmic tail were deleted. As shown in FIG. 13, a complex having the approximate size of a trimer (MW 512 kDa) was detected. No higher order oligomers were detected. These results not only suggest that the Se fragment and perhaps the full-length membrane-associated S are trimers in there native unbound state but also indicate that the dimerization site in S1 is not readily available for intertrimer interactions.

These results indicate the following: 1) the SU subunit of the SARS-CoV S glycoprotein (S1) forms dimers, 2) the dimerization domain does not overlap and is upstream of the receptor binding domain, 3) deletion of the dimerization domain abolishes fusion, 4) dimeric S1 binds receptor molecules much more efficiently than monovalent fragments containing the receptor binding domain, and 5) the soluble S ectodomain forms trimers under gel filtration conditions.

It has been previously reported that some SU subunits of class I fusion proteins (that bind receptor molecules) can form dimers including, for example, gp120 of the retrovirus HIV-1 and S1 of the coronavirus MHV. Center et al. J. Virol. 74: 4448-55 (2000); Lewicki et al. J. Biol. Chem. 277: 19727-34 (2002). Until the present work, the role of S1 dimerization for mediation of membrane fusion was unclear. It is now generally accepted that soluble ectodomains such as the gp140 protein of the HIV-1 and SIV envelope glycoproteins (Env) form trimers although dimers and tetramers can be observed. Center et al. Proc. Nat'l Acad. Sci. U.S.A. 98: 14877-82 (2001). Similarly, it appears that at least a possible fusion intermediate quaternary structure of coronaviruses including the SARS-CoV of S2 is trimeric. Liu et al. Lancet 363: 938-947 (2004); Bosch et al. Proc. Nat'l Acad. Sci. U.S.A. 101: 8455-60 (2004). In contrast, some data indicates that the MHV S2 protein is monomeric after dissociation from S1. Lewicki et al. J. Biol. Chem. 277: 19727-34 (2002). Dimer-to-trimer transitions play a critical role in the mechanism of fusion mediated by class II fusion proteins. Thus it has been proposed that changes in the quaternary structure of some coronaviruses may play a role in the fusion mechanism. Id. One should note that both the HIV-1 Env and the MHV S glycoproteins are cleaved and the SU can dissociate from the transmembrane subunit, however, such dissociation may not be important for fusion. In contrast, the SARS-CoV S is not cleaved when expressed in membrane associated or soluble form and cleavage may not be required for fusion. Thus, although the SARS-CoV S glycoprotein is a class I fusion protein, the lack of cleavage is an exception from the rule that the Envs of class I fusion proteins are cleaved presumably to confer a metastable high-energy state that could drive the fusion reaction.

This finding that the SU (S1) domain of the SARS-CoV S glycoprotein can form dimers and also forms trimers with the ectodomain of the transmembrane domain (S2) poses an interesting topological situation. Thus, if two of the monomers within a trimer also form a dimer, then the third monomer would still be free to interact with a “free” monomer from another trimer and form a dimer of the two trimers. In another scenario the orientation of each of the monomers in the trimer may not allow formation of dimers in the trimer but leave “free” binding sites for dimerization with monomers from other trimers. In this case one might expect the formation of a network of trimers. Finally, the three-dimensional structure of the trimer may not allow any interactions of the monomer dimerization sites with other monomers in the same or different trimer. The later possibility is supported by the preliminary data provided herein where higher order oligomers were not detected using the described gel filtration conditions. Under those conditions either intratrimer dimerization occurs but the third monomer conformation does not allow interactions with monomers from other trimers or such interactions are too weak to be detected, or the trimer three-dimensional structure is such that it does not allow dimerization interactions.

Data provided herein demonstrate lack of fusion after deletion of portions of the dimerization domain and indicate that the dimerization region may play a role in fusion although its mechanism may not be through dimerization interactions. In addition, under native conditions where the surface concentration of the S glycoprotein can be very high, as seen in electron micrographs, it is possible that dimerization interactions play a role in stabilizing a “network” of interacting molecules perhaps somewhat similar to networks of proteins that mediate entry of class II fusion proteins. Such networks, if any, could increase the avidity of interaction with receptor molecules and perhaps facilitate the formation of the fusion pore structure by providing a pre-assembled network of Env molecules or even provide energy to drive the fusion reaction in the absence of S cleavage that generates a high-energy metastable state.

### EXAMPLE 20

### Sera from Mice Immunized with DNA Encoding RBD Polypeptides Inhibits S-Mediated Cell Fusion

This Example illustrates that immunizing mammals with DNA encoding receptor binding domain polypeptides may prevent SARS infection.

Materials and Methods

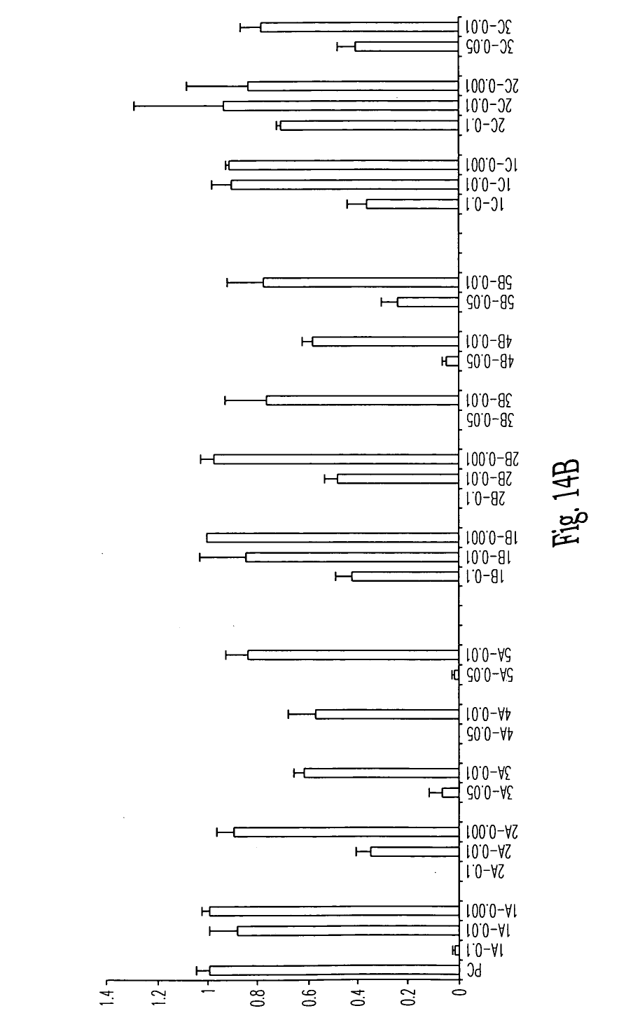

Mice were divided into three groups: group A of mice # 1 through 5 were immunized with plasmid pSecTag-SRBD that encodes for the S319-518 fragment that includes the receptor binding domain (RBD) of the spike protein; group B of mice #1 to #5 were immunized with the plasmid pEAK-10-RBD-Fc that encodes for a fusion protein of RBD (S319-518) fragment fused to Fc and group C mice #1 to #3 which were immunized with a control plasmid. Five BALB/C mice per group were immunized at day 0, day 14 and day 28. Mice received less than 2 ug DNA per immunization with a gene gun. Sera were collected at day 56. In FIG. 14A-B, the first number denotes an individual mouse, the letter denotes the respective immunization group, and the last number denotes the dilution used.

Cells (293T) were incubated with anti-sera from the immunized mice and then mixed with cells expressing S protein. Fusion was measured as described in previous Examples (see also, Xiao et al. BBRC 2003). PC denotes positive control where no serum was added. For mice #1 to #2 in each group, serum dilution factors of 10, 100, and 1000 were used. For mice #3-#5 in groups A and B, and #3 in the control group, dilution factors of 20 and 100 were used.

Results

The antibody titers for the anti-sera obtained from the mice are shown in FIG. 14A. As shown, mice immunized with DNA encoding the spike protein receptor binding domain (S319-518, groups A and B) had very high titer anti-sera-dilutions up to 1:7250 still reacted strongly to antigen in ELISA assays.

As shown in FIG. 14B, anti-sera from mice immunized with DNA encoding the spike protein receptor binding domain inhibited fusion of cells that express the S protein in a dose dependent manner. Thus, anti-sera from mouse 1A and 2A, which were immunized with DNA encoding the S receptor binding domain, substantially eliminated S-protein mediated cell fusion when used at a 1:10 dilution. Higher dilutions (1:100 and 1:1000) of this anti-sera were less effective. Similar results were observed on cell fusion inhibited by anti-sera from mouse 3A (1:20 dilution), from mouse 4A (1:20 dilution), and from mouse 5A (1:20 dilution).

These data indicate that immunizing mammals with DNA encoding S protein receptor binding domain polypeptides can raise a strong immune response against the spike protein and could prevent SARS infection. As described above, soluble fragments of the S glycoprotein that have the receptor binding domain inhibit S-mediated cell fusion (see FIG. 15).

### EXAMPLE 21

### Structure of SARS CoV Receptor Binding Domain Complexed with Neutralizing Antibodies

This Example illustrates the structural features that permit binding of SARS CoV receptor binding domain (RBD) to neutralizing antibodies.

Materials and Methods

Expression and purification of the RBD. A fragment containing residues 317˜518 from the S glycoprotein was cloned into pSecTag2B (Invitrogen) using BamHI and EcoRI restriction sites as described above. See also, Xiao et al., Biochem. Biophys. Res. Commun. 312, 1159-1164 (2003); Chakraborti et al., Virol. J 2, 73 (2005). The insert was further cloned into pAcGP67-A using the forward primer 5′ ACT GTC TAG ATG GTA CCG AGC TCG GAT CC 3′ (XbaI, SEQ ID NO:67) and the reverse primer 5′ CAG TAG ATC TCG AGG CTG ATC AGC G 3′ (BglII, SEQ ID NO:68). The pAcGP67-S was co-transfected with BaculoGold linearized baculovirus DNA into SF9 cells. High titer recombinant baculovirus stock was prepared by multiple amplifications. The protein was expressed in SF9 cells, cultured in serum free HyQ-SFX-insect medium (Hyclone), and purified from conditioned medium with HiTrap Ni chelating column. The eluted monomeric protein was concentrated, further purified with Superdex 75 10/300GL column equilibrated with PBS+0.2 M NaCl, and concentrated to 5˜10 mg/ml in PBS+0.2 M NaCl.

Selection, expression and purification of the high-affinity RBD-specific Fab m396 and its conversion to IgG1. A naïve human Fab phage display library (a total of about 10¹⁰ members) was constructed from peripheral blood B cells of 10 healthy donors and used for selection of Fabs against purified, soluble, monomeric RBD, conjugated to magnetic beads (Dynabeads M-270 Epoxy, DYNAL Inc., New Hyde Park, N.Y.). Amplified libraries of 1012 phage-displayed Fabs were incubated with 5, 3 and 1 μg of the RBD in 500 μl volume for 2 hours at room temperature during the 1^{st}, 2^{nd} an 3^{rd} rounds of biopanning, respectively. After the 3^{rd} round of biopanning, 95 clones were randomly picked from the infected TG1 cells and phage ELISA was used to identify clones of phage displaying Fabs with high binding affinity. Eight clones that bound to the RBD with A₄₅₀>1.0 were selected for further characterization. The VH and VL of these clones were sequenced. They were the same and the selected Fab was designated as m396.

The sequence of the m396 antibody heavy chain CDR3 was DTVMGGMDV (SEQ ID NO:70) and the sequence of m396 light chain CDR3 was QVWDSSSDYV (SEQ ID NO:71).

The Fab used for crystallization was first purified with H1 Trap Ni chelating column then further purified with Superdex 75 10/300GL column using PBS+0.2 M NaCl and concentrated to 10˜20 mg/ml. The Fab heavy and light chain were amplified and re-cloned in the pDR12 vector (provided by D. Burton, the Scripps Research Institute, La Jolla, Calif.) with the Fc gene fragment replaced with cDNA sequence instead of genomic DNA.

Crystallization and structure determination. The SCV RBD-Fab m396 complex was formed by mixing individual components in a 1:1 molar ratio and incubating overnight at 4° C. Crystals were obtained within 2-3 weeks by vapor diffusion technique with 15 v/v Glycerol, 20% PEG 6000, 100 mM MES sodium at pH 6.5 only for 1:2 ratio of complex and reservior solution. A data set up to 2 Å resolution was collected at the Southeast Regional Collaborative Access Team (SER-CAT) beamline facility 22-ID of Advanced Photon Source (APS), Argonne National Laboratory. Data processing was carried out with the HKL2000 program suite (Otwinowski, Z. & Minor, W. Processing of X-ray diffraction data collected in oscillation mode. Methods Enzymol 276, 307-326 (1997)).

SCV RBD/Fab

m396

Crystal data and processing statistics are summarized in Table 2.

Data Collection, Processing and Refinement Statistics.
Data collection

| Space group | P2₁ |
|---|---|
| Cell dimensions | |
| a, b, c (Å) | 64.7, 68.6, 80.1 |
| α, β, γ (°) | 90.0, 98.1, 90.0 |
| Resolution range (Å) | 30.0-2.30 |
| R_{sym} or R_{merge} (%) | 0.060 (0.263) |
| I/σ I | 16.1 (2.2) |
| Completeness (%) | 91.0 (72.8) |
| Redundancy | 3.0 (2.0) |

Refinement

| | |
|---|---|
| Resolution (Å) | 30-2.3 |
| No. reflections | 27719 |
| R_{work/}R_{free} | 19.8/26.1 |
| R.m.s deviations | |
| Bond lengths (Å) | 0.000 |
| Bond angles (°) | 0.0 |

The structure was solved by molecular replacement with PHASER (see Storoni et al., Acta Crystallogr. D. Biol. Crystallogr. 60, 432-438 (2004)) by using the SCV RBD from the receptor complex (PDB code 2AJF) and four independent domains, V_{H}, V_{L}, C_{H} and C_{L}, separately used as search models (7.9° of rotation angle between C_{H} and C_{L} domains was observed in the Fab m396). The RBM (˜60 residues, 430-490) of SCV RBD and most of the CDRs of Fab models were not included in the search models and built from the electron density. The complex was refined with CNS (see Brunger et al. Structure. 5, 325-336 (1997)) and final model was refined to 2.3 Å resolution. A total of 299 water molecules, a phosphate ion and one N-linked glucosamine moiety at Asn330 were added at the final stages of refinements. The final R and R_{free} were 19.8 and 26.1, respectively (Table 2).

Results

The structure of the SCV RBD in complex with the potent neutralizing antibody Fab m396, described here, identifies a major neutralization determinant, its relation to the receptor recognition, elucidates structural mechanisms of neutralization, and provides insights in the mechanisms of SCV entry.

The major epitopes useful for generating anti-SARS CoV antibodies include peptidyl sequence GFYTTTGIGYQ (SEQ ID NO:69) at positions 482-492 of the S protein.

The RBD structure complexed with Fab m306 is similar to that of the RBD in complex with ACE2 (Li et al., Science 309, 1864-1868 (2005)) although the higher resolution allowed the identification of some of the residues that were disordered or not located previously. The RBD consists of a core, which includes a five (β1-β4 and β7) stranded anti-parallel sheet, and a long extended loop with a two-stranded anti-parallel (β5-β6) sheet at the middle which is attached to the core. The antibody-complexed RBD structure contains eight cysteines that form three disulphide bridges in the core and one in the extended loop.

The value of the root mean square deviation between the C-_{α} atom positions of the RBD structure complexed with the antibody and the structure complexed with the receptor is 1.3 Å. The antibody-bound RBD structure is relatively well defined because of the high resolution and it includes the previously unresolved residues 376 to 381 and 503 to 511 which unambiguously locate an additional disulphide bridge between 378 and 511. Thus, peptidyl sequences NDLCFSNV (SEQ ID NO:70, S protein positions 375-382) and FELLNAPATVCG (SEQ ID NO:71, S protein positions 501-512) may be involved in establishing a S protein conformation that facilitates formation and maintenance of a stable complex between the S protein RBD and neutralizing antibodies thereto.

The shape correlation statistical parameter (S_{c}, Lawrence & Colman, J. Mol. Biol. 234: 946-50 (1993)), a measure of geometric fit between two juxtaposed surfaces with the maximum value of 1, calculated for the RBD-antibody interface is 0.66, which indicates a high degree of shape complementarity. A total surface area of 1760 Å² is buried at the interface of the complex with nearly equal contributions of 870 Å² from the RBD and 890 Å² from the antibody as determined by a 1.4 Å probe. The antibody-binding β6-β7 loop alone accounts for 63% of the RBD-antibody interface, which indicates the dominant role of the loop residues involved in the binding. The heavy chain CDRs contributes 66% of the total surface of the antibody combining site. The size of the binding interface is close to the average for other antigen-antibody complexes (Davies & Cohen, Proc. Natl. Acad. Sci. U.S.A. 93, 7-12 (1996)).

The Fab m396 antibody mainly recognizes 10 residues at positions 482 to 491 along the β6-β7 loop that prominently protrudes from the RBD surface. This loop contacts four of the CDRs (complementarity-determining-regions) of the Fab m396: H1, H2, H3 and L3. The four CDRs form a shallow cleft on the surface of the antibody variable regions providing a deep binding pocket into which the β6-β7 loop tightly fits. Most of the residues of the β6-β7 loop interact with Fab m396 at the binding pocket. In particular, residues Ile489 and Tyr491 penetrate into the deep pocket on the surface of the antibody-combining site. Fifteen to seventeen residues from the RBD and the Fab m396 participate in the interactions and form the RBD-antibody interface defined within the limit of 3.5 Å contact distance between the two molecules. These residues are identified in Table 3, and include the following S protein RBD residues: Thr-363, Lys-365, Lys-390, Gly-391, Asp-392, Arg-395, Tyr-436, Arg-426, Gly-482, Tyr-484, Thr-485, Thr-486, Thr-487, Gly-488, Ile489, Tyr491, Gln-492 and Tyr-494.

The intermolecular interactions existing across the binding interface have contributions from van der Waals contacts, and direct and water-mediated hydrogen bonds (Tables 3 and 4).

TABLE 3
Contacts between residues at the SCV RBD/Fab m396 interface.

| | | Hydrogen bonds^{b} | | |
|---|---|---|---|---|
| | | | | Dis- |
| van der Waals contacts^{a} | | | | tance |
| Antibody | SCV RBD | Antibody | SCV RBD | (Å) |
| H1 | | H1 | | |
| Ser^{H31} | Tyr^{S484} (3), Thr^{S486} | Ser^{H31} (O) | Thr^{S486} Oγ1) | 2.61 |
| | (7), Thr^{S487} (3) | Thr^{H33}(Oγ1) | Gly^{S488} (N) | 2.67 |
| Tyr^{H32} | Thr^{S486} (2) | | | |
| Thr^{H33} | Thr^{S487} (3), Gly^{S488} | | | |
| | (4), Tyr^{S491} (1) | | | |
| H2 | | H2 | | |
| Gly^{H50} | Tyr^{S491} (2) | Thr^{H52} (Oγ1) | Tyr^{S491} (Cγ) | 3.19 |
| Ile^{H51} | Tyr^{S491} (3) | Thr^{H52} (Oγ1) | Tyr^{S491} (Cδ1) | 3.26 |
| Thr^{H52} | Tyr^{S487} (1), Tyr^{S491} | Thr^{H52} (Oγ1) | Tyr^{S491} (Cδ2) | 3.16 |
| | (4) | Thr^{H52} (Oγ1) | Tyr^{S491} (Cε1) | 3.33 |
| Ile^{H53} | Tyr^{S484} (4) | Thr^{H52} (Oγ1) | Tyr^{S491} (Cε2) | 3.24 |
| Leu^{H54} | Tyr^{S436} (1), | Thr^{H52} (Oγ1) | Tyr^{S491} (Cζ) | 3.32 |
| | Gly^{S482}(3), Tyr^{S484} | Ile^{H56} (Cδ1) | Lys^{S390} (Nζ) | 3.01 |
| | (1) | Asn^{H58} (Nδ2) | Tyr^{S491} (OH) | 2.81 |
| Ile^{H56} | Lys^{S390} (1), Tyr^{S491} | Asn^{H58} (Nδ2) | Tyr^{S491} (Cζ) | 3.11 |
| | (4) | | | |
| Asn^{H58} | Asp^{S392}(6), | | | |
| | Tyr^{S491}(7) | | | |
| H3 | | H3 | | |
| Val^{H97} | Arg^{S426} (6), | Val^{H97} (O) | Gln^{S492} (Nε2) | 2.76 |
| | Thr^{S485} (6), | | | |
| | Gln^{S492} (4) | | | |
| L3 | | L3 | | |
| Trp^{L91} | Gly^{S391}(1), | Ser^{L94} (N) | Lys^{S365} (Nζ) | 3.06 |
| | Asp^{S392}(1), Ile^{S489} | Asp^{L95} (Oδ1) | Arg^{S395} (NH2) | 3.00 |
| | (7), Tyr^{S494} (1) | | | |
| Ser^{L93} | Thr^{S363} (2), | | | |
| | Arg^{S395} (4) | | | |
| Ser^{L94} | Lys^{S365} (1), | | | |
| | Arg^{S395} (1) | | | |

^{a}Van der Waals contacts have interatomic distance ≦4.0 Å
^{b}hydrogen bonds criteria based on donor-acceptor distances (≦3.5 Å)

TABLE 4
Water-mediated hydrogen bonds^{b}

| | | |
|---|---|---|
| Antibody | SCV RBD | Distance (Å) |

H2

| | | |
|---|---|---|
| Asn^{H58} (Oδ1) | Lys^{S390} (Nζ)) | 2.55, 2.76 |
| Asn^{H58} (Nδ2) | Asp^{S392} (Oδ2) | 2.53, 2.55 |

L1

| | | |
|---|---|---|
| Ser^{L30} (Cα) | Thr^{S359} (O) | 3.14, 2.62 |

L3

| | | |
|---|---|---|
| Trp^{L91} (Nε1) | Asp^{S392} (Oδ1) | 2.88, 2.67 |
| Ser^{L93} (O) | Gly^{S391} (O) | 2.68, 2.59 |
| Tyr^{L96} (OH) | Gly^{S490} (N) | 2.83, 2.73 |

^{b}hydrogen bonds criteria based on donor-acceptor distances (≦3.5 Å)

The details of the buried surface area at the interface between the antibody and the SCV RBD in the complex are given in Table 5.

TABLE 5
Buried surface area (Å²) of the residues at the SCV RBD/Fab
m396 interface.

| | Buried |
|---|---|
| | Area (Å²) |
| Antibody | |
| H1 | |
| Ser^{H31} | 58.14 |
| Tyr^{H32} | 19.42 |
| Thr^{H33} | 36.39 |
| H2 | |
| Gly^{H50} | 2.0 |
| Ile^{H51} | 2.31 |
| Thr^{H52} | 20.72 |
| Ile^{H53} | 34.36 |
| Leu^{H54} | 88.19 |
| Ile^{H56} | 40.69 |
| Asn^{H58} | 52.03 |
| H3 | |
| Val^{H97} | 108.12 |
| L3 | |
| Trp^{L91} | 99.32 |
| Ser^{L93} | 93.11 |
| Ser^{L94} | 45.4 |
| Asp^{L95} | 28.26 |
| SCV RBD | |
| β2-strand | |
| Thr^{S363} | 13.78 |
| Lys^{S365} | 36.94 |
| 3₁₀-helix | |
| Lys^{S390} | 31.33 |
| Gly^{S391} | 15.13 |
| Asp^{S392} | 38.29 |
| Arg^{S395} | 86.08 |
| Other | |
| residues | |
| Arg^{S426} | 33.18 |
| Thr^{S436} | 13.86 |
| β6-β7 | |
| loop | |
| Gly^{S482} | 20.29 |
| Tyr^{S484} | 55.3 |
| Thr^{S485} | 4.01 |
| Thr^{S486} | 55.41 |
| Thr^{S487} | 52.85 |
| Gly^{S488} | 46.39 |
| Ile^{S489} | 96.93 |
| Tyr^{S491} | 122.2 |
| Gln^{S492} | 15.42 |
| Tyr^{S494} | 12.74 |

The key interactions in the SCV RBD-antibody complex are mostly between the β6-β7 loop of the RBD and the four CDRs, H1, H2, H3 and L3 of the antibody Fab m396. These interactions are clearly defined in an electron density map (not shown). Thus, H1 makes contacts with the hydrophobic residues Tyr484, Thr486 and Thr487. In particular, Thr33 of H1 is in direct contact with the amide nitrogen atom of the Gly488 residue in the RBD main-chain. This type of interaction is also found in the RBD-ACE2 complex where the amide of S protein RBD Gly488 is engaged in main-chain hydrogen bonding with the carbonyl of Lys35 in ACE2. Compared to other CDRs, H2 has a dominant role in the RBD binding and uses large number of residues to make contacts; the most conspicuous feature is the burial of Tyr491 of the RBD (122 Å²) in the shallow cleft rendered by the H2, where the amino group of Asn58 in H2 contacts the phenolic oxygen atom of the RBD Tyr491. Another important interaction is between H2 Thr52 and the phenyl ring of Tyr491. Val97 is the only residue from the H3 region that is involved in the RBD interaction; however, it buries the largest surface area (108 Å²) of all CDR residues. Thus, the carbonyl oxygen atom of Val97 makes a strong hydrogen bond with the amino group nitrogen atom of the RBD Gln492 within a distance of 2.7 Å. Contacts between such main-chain and side-chain residues involving directional hydrogen bonds, as in H1, H2 and H3, play an important role in determining the relative orientation of the RBD and the antibody in the complex, and contribute to the specificity of the interactions.

The S_{c} parameter calculated for the heavy chain-RBD interaction has a high value of 0.74, which suggests a highly correlated interfacial geometry for the heavy chain-RBD recognition. L3-RBD interaction involves water-mediation and other minor binding sites including Arg395 of RBD (Tables 3 and 4). The residue Trp91 of L3 stacks with the aromatic residue Ile489, which is a major hot spot in the RBD; each of the Trp91 and Ile489 residues buries a surface area of about 100 Å² at the interface.

The minor binding sites on RBD include residues in β2 (Thr363 and Lys365), 3₁₀ helix followed by β3 (Lys390, Gly391, Asp392 and Arg395) and two residues at Arg426 and Tyr436. Apart from the minor contributions of these residues to antibody binding, most of them have significant roles in stabilizing the conformation of the β6-β7 loop. Particularly, different types of hydrogen bonds, including those between the nitrogen atom of Gly391 and the carbonyl oxygen atoms of Gly490 and Gln492, the amino group of Arg426 and the backbone carbonyl of Thr485 and those between the phenolic oxygen atoms of Tyr436 and Tyr484 stabilize the β6-β7 loop conformation in the RBD.

The RBD-antibody binding interface has two major characteristic features: first, the high level of complementarity between the interacting surfaces and second, the anchoring of the major hotspot RBD residue Tyr491 into the antibody combining site. The RBD-antibody interactions are produced by abundant hydrophobic residues, and networks involving hydrophilic and polar residues. The two hot spots, Ile 489 and Tyr491, of the RBD β6-β7 loop form a well protruding ridge, while the antibody binding pocket includes cavities in a shallow cleft mostly formed by the heavy chain. Thus, the paratope and the epitope structures are highly complimentary, which could be a major factor for the high affinity of their interaction. Another characteristic feature of the RBD-antibody interaction is the insertion of the RBD Tyr491 into the bottom of the binding pocket at the antibody combining site where Thr52 and Asn58 of the H2 interact with Tyr491 in a specific manner. The Tyr491 residue is strongly held in between the two residues in such a way that the phenolic hydroxyl group of Tyr491 forms a hydrogen bond with the nitrogen atom of the Asn52 side chain, while the phenyl ring of Tyr491 acts as a perfect hydrogen-bond acceptor for the Thr52 side chain oxygen atom. The structural basis of preferential recognition of these two H2 residues, which line up the combining-site pocket in the antibody, by the Tyr491 involves specificities of the side chains, which are a unique structural feature of the RBD-antibody Fab m396 recognition.

The comparison of the RBD-ACE2 and the RBD-m396 structures provided important clues for understanding the molecular basis of antibody-mediated neutralization and the mechanisms of SCV entry. The antibody and the receptor occupy a common region consisting of the β6-β7 loop (Thr484, Thr486, Thr487, G488, and Y491) and Arg426 on RBD. These common residues were found critical for the RBD binding to the antibody and to ACE2. The major difference between the antibody and the receptor binding is related to specific residues defining the receptor binding determinants other than the common binding region (α6-α7 loop). The neutralizing determinants are located contiguously in one major segment of β6-β7 loop while the receptor ACE2 have determinants over most of the extended loop appearing on the top of the RBD. The high level of overlap of the heavy chain with the ACE2 centering on the β6-β7 loop shows the common determinants for the neutralization and receptor recognition. These observations demonstrate that the antibody neutralizes SARX-CoV by competition for the same critical residues in the β6-β7 loop of the S protein RBD and by steric hindrance that blocks the receptor binding site on RBD.

Recently, bats were reported as a reservoir of SARS-like coronaviruses. Li, W. et al. Bats are natural reservoirs of SARS-like coronaviruses. Science 310, 676-679 (2005); Lau, S. K. et al. Severe acute respiratory syndrome coronavirus-like virus in Chinese horseshoe bats. Proc. Natl. Acad. Sci. U S. A 102, 14040-14045 (2005). The sequences of human and civet isolates differ greatly from those of bat isolates although they all are phylogenetically related. Notably, the residues Arg426, Ile489 and Tyr491 of the SCV RBD, which are involved in the antibody binding to RBD, are conserved among the known bat isolates, indicating a potential neutralizing activity of m396 although this possibility should be evaluated experimentally.

The RBD structure in the complex with antibody was unexpectedly similar to the ACE2-complexed RBD structure. Although the structure of unliganded RBD is currently unknown, it is likely quite similar to the RBD structure bound to the antibody m396 or ACE2. However, it is unlikely that binding of ACE2 and m396 would induce exactly the same conformational changes in the RBD to generate essentially the same structure of the RBD, especially in light of their overlapping but different binding sites and other molecular specificities of their binding. The unexpected similarity in binding structures challenges the current paradigm that the SARS CoV entry is through an ACE2-activating mechanism, although it is possible that membrane-associated ACE2 could induce conformational changes in the trimeric S glycoprotein through multivalent binding. Another possibility is that ACE2 functions by binding specifically to the S glycoprotein followed by binding to coreceptor(s) that can induce conformational changes activating the fusogenic machinery of the S glycoprotein.

These results have implications for development of vaccines and therapeutics against SARS CoV. Moreover, these results enhance our understanding of the mechanisms of antibody-mediated virus neutralization and virus entry. The newly identified antibody, m396, itself may have therapeutic potential; it is currently being evaluated for its potency against infectious virus and will be tested in animal models. Based on its structure (the structure of unbound m396 was also determined, which does not significantly differ from the bound one (data not shown)) one could design other therapeutic modalities.

The structure of the antibody epitope could be used for design of vaccine immunogens that are likely to elicit m396 or m396-like antibodies (a retrovaccinology approach, Burton, D. R. Antibodies, viruses and vaccines. Nat. Rev. Immunol. 2, 706-13 (2002)). Especially attractive is the potential use of the main neutralizing determinant, the β6-β7 loop, and constraint peptides based on its sequence as vaccine immunogens. Its protruding nature, exposure and easy access by antibodies suggest a critical role in neutralization mechanisms, and it is likely that it also binds other antibodies in addition to the receptor ACE2 and m396.

### REFERENCES

D. S. Dimitrov, Cell biology of virus entry, Cell 101 (2000) 697-702.
D. S. Dimitrov, Virus entry: molecular mechanisms and biomedical applications, Nat. Rev. Microbiol. 2 (2004) 109-122.
K. V. Holmes, SARS-associated coronavirus, N. Engl. J. Med. 348 (2003) 1948-1951.
M. M. Lai, D. Cavanagh, The molecular biology of coronaviruses, Adv. Virus Res. 48 (1997) 1-100.
T. G. Ksiazek, D. Erdman, C. S. Goldsmith, S. R. Zaki, T. Peret, S. Emery, S. Tong, C. Urbani, J. A. Corner, W. Lim, P. E. Rollin, S. F. Dowell, A. E. Ling, C. D. Humphrey, W. J. Shieh, J. Guarner, C. D. Paddock, P. Rota, B. Fields, J. DeRisi, J. Y. Yang, N. Cox, J. M. Hughes, J. W. LeDuc, W. J.
Bellini, L. J. Anderson, A novel coronavirus associated with severe acute respiratory syndrome, N. Engl. J. Med. 348 (2003) 1953-1966.
P. A. Rota, M. S. Oberste, S. S. Monroe, W. A. Nix, R. Campagnoli, J. P. Icenogle, S. Penaranda, B. Bankamp, K. Maher, M. H. Chen, S. Tong, A. Tamin, L. Lowe, M. Frace, J. L. DeRisi, Q. Chen, D. Wang, D. D. Erdman, T. C. Peret, C. Burns, T. G. Ksiazek, P. E. Rollin, A. Sanchez, S. Li.ck, B. Holloway, J. Limor, K. McCaustland, M. Olsen-Rasmussen, R. Fouchier, S. Gunther, A. D. Osterhaus, C. Drosten, M. A. Pallansch, L. J. Anderson, W. J. Bellini, Characterization of a novel coronavirus associated with severe acute respiratory syndrome, Science 300 (2003) 1394-1399.
M. A. Marra, S. J. Jones, C. R. Astell, R. A. Holt, A. Brooks-Wilson, Y. S. Butterfield, J. Khattra, J. K. Asano, S. A. Barber, S. Y. Chan, A. Cloutier, S. M. Coughlin, D. Freeman, N. Girn, O. L. Gri.th, S. R. Leach, M. Mayo, H. McDonald, S. B. Montgomery, P. K. Pandoh, A. S. Petrescu, A. G. Robertson, J. E. Schein, A. Siddiqui, D. E. Smailus, J. M. Stott, G. S. Yang, F. Plummer, A. Andonov, H. Artsob, N. Bastien, K. Bernard, T. F. Booth, D. Bowness, M. Czub, M. Drebot, L. Fernando, R. Flick, M. Garbutt, M. Gray, A. Grolla, S. Jones, H. Feldmann, A. Meyers, A. Kabani, Y. Li, S. Normand, U. Stroher, G. A. Tipples, S. Tyler, R. Vogrig, D. Ward, B. Watson, R. C. Brunham, M. Krajden, M. Petric, D. M. Skowronski, C. Upton, R. L. Roper, The genome sequence of the SARS-associated coronavirus, Science 300 (2003) 1399-1404.
T. M. Gallagher, M. J. Buchmeier, Coronavirus spike proteins in viral entry and pathogenesis, Virology 279 (2001) 371-374.
F. Kunkel, G. Herrler, Structural and functional analysis of the surface protein of human coronavirus OC43, Virology 195 (1993) 195-202.
A. Bonavia, B. D. Zelus, D. E. Wentworth, P. J. Talbot, K. V. Holmes, Identi.cation of a receptor-binding domain of the spike glycoprotein of human coronavirus HCoV-229E, J. Virol. 77 (2003) 2530-2538.
W. Li, M. J. Moore, N. Vasilieva, J. Sui, S. K. Wong, M. A. Berne, M. Somasundaran, J. L. Sullivan, K. Luzuriaga, T. C. Greenough, H. Choe, M. Farzan, Angiotensin-converting enzyme 2 is a functional receptor for the SARS coronavirus, Nature 426: 450-54 (2003).
P. L. Earl, B. Moss, Mutational analysis of the assembly domain of the HIV-1 envelope glycoprotein, AIDS Res. Hum. Retroviruses. 9 (1993) 589-94.
R. J. Center, P. Schuck, R. D. Leapman, L. O. Arthur, P. L. Earl, B. Moss, J. Lebowitz, Oligomeric structure of virion-associated and soluble forms of the simian immunodeficiency virus envelope protein in the prefusion activated conformation, Proc. Natl. Acad. Sci. U.S.A 98 (2001) 14877-14882.
R. J. Center, P. L. Earl, J. Lebowitz, P. Schuck, B. Moss, The human immunodeficiency virus type 1 gp120 V2 domain mediates gp41-independent intersubunit contacts, J. Virol. 74 (2000) 4448-4455.
D. N. Lewicki, T. M. Gallagher, Quaternary structure of coronavirus spikes in complex with carcinoembryonic antigen-related cell adhesion molecule cellular receptors, J. Biol. Chem. 277 (2002) 19727-19734.
B. Tripet, M. W. Howard, M. Jobling, R. K. Holmes, K. V. Holmes, R. S. Hodges, Structural characterization of the SARS-coronavirus spike S fusion protein core, J. Biol. Chem. 279 (2004) 20836-20849.
J. Zhu, G. Xiao, Y. Xu, F. Yuan, C. Zheng, Y. Liu, H. Yan, D. K. Cole, J. I. Bell, Z. Rao, P. Tien, G. F. Gao, Following the rule: formation of the 6-helix bundle of the fusion core from severe acute respiratory syndrome coronavirus spike protein and identification of potent peptide inhibitors, Biochem. Biophys. Res. Commun. 319 (2004) 283-288.
B. J. Bosch, B. E. Martina, Z. R. van der, J. Lepault, B. J. Haijema, C. Versluis, A. J. Heck, R. de Groot, A. D. Osterhaus, P. J. Rottier, Severe acute respiratory syndrome coronavirus (SARS-CoV) infection inhibition using spike protein heptad repeat-derived peptides, Proc. Natl. Acad. Sci. U.S.A 101 (2004) 8455-8460.
S. Liu, G. Xiao, Y. Chen, Y. He, J. Niu, C. R. Escalante, H. Xiong, J. Farmar, A. K. Debnath, P. Tien, S. Jiang, Interaction between heptad repeat 1 and 2 regions in spike protein of SARS-associated coronavirus: implications for virus fusogenic mechanism and identification of fusion inhibitors, Lancet 363 (2004) 938-947.
B. J. Bosch, Z. R. van der, C. A. de Haan, P. J. Rottier, The coronavirus spike protein is a class I virus fusion protein: structural and functional characterization of the fusion core complex, J. Virol. 77 (2003) 8801-8811.
H. Bisht, A. Roberts, L. Vogel, A. Bukreyev, P. L. Collins, B. R. Murphy, K. Subbarao, B. Moss, Severe acute respiratory syndrome coronavirus spike protein expressed by attenuated vaccinia virus protectively immunizes mice, Proc. Natl. Acad. Sci. U.S.A 101 (2004) 6641-6646.
G. J. Babcock, D. J. Esshaki, W. D. Thomas, Jr., D. M. Ambrosino, Amino acids 270 to 510 of the severe acute respiratory syndrome coronavirus spike protein are required for interaction with receptor, J. Virol. 78 (2004) 4552-4560.
G. Simmons, J. D. Reeves, A. J. Rennekamp, S. M. Amberg, A. J. Piefer, P. Bates, Characterization of severe acute respiratory syndrome-associated coronavirus (SARS-CoV) spike glycoprotein-mediated viral entry, Proc. Natl. Acad. Sci. U.S.A 101 (2004) 4240-4245.
S. K. Wong, W. Li, M. J. Moore, H. Choe, M. Farzan, A 193-amino acid fragment of the SARS coronavirus S protein efficiently binds angiotensin-converting enzyme 2, J. Biol. Chem. 279 (2004) 3197-3201.
Smith, A. E. & Helenius, A. How viruses enter animal cells. Science 304, 237-242 (2004).
Peiris, J. S. et al. Coronavirus as a possible cause of severe acute respiratory syndrome. Lancet 361, 1319-1325 (2003).
Drosten, C. et al. Identification of a novel coronavirus in patients with severe acute respiratory syndrome. N. Engl. J. Med. 348, 1967-1976 (2003).
Xiao, X., Chakraborti, S., Dimitrov, A. S., Gramatikoff, K. & Dinitrov, D. S. The SARS-CoV S glycoprotein: expression and functional characterization. Biochem. Biophys. Res. Commun. 312, 1159-1164 (2003).
Xiao, X. & Dimitrov, D. S. The SARS-CoV S glycoprotein. Cell Mol. Life Sci. 61, 2428-2430 (2004).
He, Y. et al. Receptor-binding domain of SARS-CoV spike protein induces highly potent neutralizing antibodies: implication for developing subunit vaccine. Biochem. Biophys. Res. Commun. 324, 773-781 (2004).
Zhang, M. Y., Choudhry, V., Xiao, X. & Dimitrov, D. S. Human monoclonal antibodies to the S glycoprotein and related proteins as potential therapeutics for SARS. Curr. Opin. Mol. Ther. 7, 151-156 (2005).
Jiang, S., He, Y. & Liu, S. SARS vaccine development. Emerg. Infect. Dis. 11, 1016-1020 (2005).
He, Y., Lu, H., Siddiqui, P., Zhou, Y. & Jiang, S. Receptor-binding domain of severe acute respiratory syndrome coronavirus spike protein contains multiple conformation-dependent epitopes that induce highly potent neutralizing antibodies. J Immunol. 174, 4908-4915 (2005).
He, Y. et al. Identification of a critical neutralization determinant of severe acute respiratory syndrome (SARS)-associated coronavirus: importance for designing SARS vaccines. Virology 334, 74-82 (2005).
Chen, Z. et al. Recombinant modified vaccinia virus Ankara expressing the spike glycoprotein of severe acute respiratory syndrome coronavirus induces protective neutralizing antibodies primarily targeting the receptor binding region. J. Virol. 79, 2678-2688 (2005).
Yi, C. E., Ba, L., Zhang, L., Ho, D. D. & Chen, Z. Single amino acid substitutions in the severe acute respiratory syndrome coronavirus spike glycoprotein determine viral entry and immunogenicity of a major neutralizing domain. J. Virol. 79, 11638-11646 (2005).
Li, F., Li, W., Farzan, M. & Harrison, S. C. Structure of SARS coronavirus spike receptor-binding domain complexed with receptor. Science 309, 1864-1868 (2005).
Burton, D. R. Antibodies, viruses and vaccines. Nat. Rev. Immunol. 2, 706-713 (2002).
Chakraborti, S., Prabakaran, P., Xiao, X. & Dimitrov, D. S. The SARS coronavirus S glycoprotein receptor binding domain: fine mapping and functional characterization. Virol. J2, 73 (2005).
Nybakken, G. E. et al. Structural basis of West Nile virus neutralization by a therapeutic antibody. Nature 437, 764-769 (2005).
Lawrence, M. C. & Colman, P. M. Shape complementarity at protein/protein interfaces. J Mol Biol. 234, 946-950 (1993).
Davies, D. R. & Cohen, G. H. Interactions of protein antigens with antibodies. Proc. Natl. Acad. Sci. U.S.A. 93, 7-12 (1996).
Li, W. et al. Bats are natural reservoirs of SARS-like coronaviruses. Science 310, 676-679 (2005).
Lau, S. K. et al. Severe acute respiratory syndrome coronavirus-like virus in Chinese horseshoe bats. Proc. Natl. Acad. Sci. U.S. A 102, 14040-14045 (2005).
Otwinowski, Z. & Minor, W. Processing of X-ray diffraction data collected in oscillation mode. Methods Enzymol 276, 307-326 (1997).
Storoni, L. C., McCoy, A. J. & Read, R. J. Likelihood-enhanced fast rotation functions. Acta Crystallogr. D. Biol. Crystallogr. 60, 432-438 (2004).
Brunger, A. T., Adams, P. D. & Rice, L. M. New applications of simulated annealing in X-ray crystallography and solution NMR. Structure. 5, 325-336 (1997).

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “a host cell” includes a plurality (for example, a culture or population) of such host cells, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A polypeptide consisting of any one of SEQ ID NOs: 13, 14, 15, 20-59, 61-63, 69, or a combination thereof, wherein the polypeptide can produce a humoral or cellular immune response when used to inoculate a mammal.

2. The polypeptide of claim 1, consisting of any one of SEQ ID NOs: 13, 14, 15, 25, 34, 46, 51, 52, 56, 57, 58, 59, 61, 62, 63, 66 or 69.

3. The polypeptide of claim 1, wherein the polypeptide is soluble in an aqueous solution.

4. The polypeptide of claim 1, wherein mammal is a human.

5. The polypeptide of claim 1, wherein the polypeptide is amino-terminally or carboxyl-terminally blocked.

6. A coupled protein comprising a carrier protein coupled to a second polypeptide consisting of any one of SEQ ID NOs: 13, 14, 15, 20-59, 61-63, 66 or 69.

7. The coupled protein of claim 6, wherein the carrier protein is soluble in an aqueous solution.

8. The coupled protein of claim 6, wherein the carrier protein is selected from the group consisting of bovine serum albumin, keyhole limpet hemacyanin, ovalbumin, mouse serum albumin, rabbit serum albumin.

9. The coupled protein of claim 6, wherein the coupled protein produces a humoral or a cellular immune response when used to inoculate a mammal.

10. The coupled protein of claim 9, wherein the mammal is a human.

11. An immunopeptide comprising the polypeptide of claim 1 coupled to arsanilic acid, sulfanilic acid, an acetyl group, or a picryl group.

12. The immunopeptide of claim 11, wherein the immunopeptide produces a humoral or a cellular immune response when used to inoculate a mammal.

13. The immunopeptide of claim 12, wherein the mammal is a human.

14. A peptidomimetic of the polypeptide of claim 1.

15. A composition comprising a carrier and an effective amount of the polypeptide of claim 1.

16. The composition of claim 15, wherein the carrier is an adjuvant selected from the group consisting of aluminum hydroxide, lipid A, killed bacteria, polysaccharide, mineral oil, Freund's incomplete adjuvant, Freund's complete adjuvant, aluminum phosphate, iron, zinc, a calcium salt, acylated tyrosine, an acylated sugar, a cationically derivatized polysaccharide, an anionically derivatized polysaccharide, a polyphosphazine, a biodegradable microsphere, a monophosphoryl lipid A, and quil A.

17. The composition of claim 15, wherein the polypeptide is amino-terminally or carboxyl-terminally blocked.

18. The composition of claim 15, wherein the effective amount of the polypeptide is effective for treatment of SARS-CoV infection.

19. The composition of claim 15, wherein the effective amount of the polypeptide is effective for inhibition of SARS-CoV fusion with, or entry into, mammalian cells.

20. A nucleic acid segment that encodes the polypeptide of claim 1.

21. An expression cassette comprising a promoter that is operably linked to the nucleic acid segment of claim 20.

22. The expression cassette according to claim 21, wherein the promoter is a constitutive promoter or a regulated promoter.

23. A nucleic acid construct comprising a vector that comprises a nucleic acid segment that encodes a polypeptide consisting of any one of SEQ ID NOs: 13, 14, 15, 20-59, 61-63, 66 or 69, or the expression cassette according to claim 21.

24. The nucleic acid construct according to claim 23, wherein the vector is selected from the group consisting of a plasmid, a cosmid, a yeast artificial chromosome, a bacterial artificial chromosome, an F-factor, a virus, an expression vector, and a phagemid.

25. A composition comprising a pharmaceutical carrier and a nucleic acid segment that encodes a polypeptide consisting of any one of SEQ ID NOs: 13, 14, 15, 20-59, 61-63, 66, 69 or an expression cassette according to claim 21.

26. The composition of claim 25, wherein the effective amount of the nucleic acid segment is effective for treatment of SARS-CoV infection.

27. The composition of claim 25, wherein the effective amount of the nucleic acid segment is effective for inhibition of SARS-CoV fusion with, or entry into, mammalian cells.

28. A recombinant virus comprising a viral vector and a nucleic acid segment that encodes the polypeptide consisting of any one of SEQ ID NOs: 13, 14, 15, 20-59, 61-63, 66 or 69; or the expression cassette according to claim 21.

29. The recombinant virus of claim 28, wherein the viral vector is selected from the group consisting of vaccinia virus, canarypox, adenovirus, and herpes virus.

30. A composition comprising a pharmaceutical carrier and an effective amount of the recombinant virus of claim 28.

31. The composition of claim 30, wherein the effective amount of the recombinant virus is effective for treatment or prevention of SARS-CoV infection.

32. A composition comprising a pharmaceutical carrier and a microorganism that comprises a nucleic acid segment encoding a polypeptide consisting of any one of SEQ ID NOs: 13, 14, 15, 20-59, 61-63, 66 or 69; or the expression cassette according to claim 21.

33. The composition of claim 32, wherein the microorganism is selected from the group consisting of Salmonella and Listeria monocytogenes.

34. An immunological composition comprising a pharmaceutical carrier and a DNA vector into which is inserted the expression cassette of claim 21.

35. The composition of claim 34, wherein the vector is selected from the group consisting of a plasmid, a cosmid, a yeast artificial chromosome, a bacterial artificial chromosome, an F-factor, a virus, and a phagemid.

36. The composition of claim 34, wherein the composition further comprises a myonecrotic agent.

37. The composition of claim 36, wherein the myonecrotic agent is bupivicaine or cardiotoxin.

38. An antibody that binds specifically to a polypeptide comprising an amino acid sequence consisting of any one of SEQ ID NOs: 13, 14, 15, 20-59, 61-63, 66 or 69.

39. The antibody according to claim 38, wherein the antibody specifically binds to an epitope consisting of SEQ ID NO:61, 62, 63, 66 or 69.

40. The antibody according to claim 38, wherein the antibody is a monoclonal antibody, a polyclonal antibody, a single-chain antibody, an antigen-binding antibody fragment, or a humanized antibody.

41. The antibody according to claim 40, wherein the antigen-binding antibody fragment is an scFv, Fv, Fab′, Fab, diabody, linear antibody or F(ab′)₂.

42. The antibody according to claim 38, wherein the antibody is coupled to a detectable tag.

43. The antibody according to claim 42, wherein the detectable tag is a fluorescent protein, a fluorescent marker, a radiolabel, an enzyme, or an affinity tag.

44. The antibody according to claim 38, wherein the antibody is coupled to a toxin.

45. The antibody according to claim 44, wherein the toxin is an A chain toxin, a ribosome inactivating protein, α-sarcin, gelonin, aspergillin, restrictocin, a ribonuclease, an epipodophyllotoxin, diphtheria toxin, Pseudomonas exotoxin, ricin, doxorubicin, daunorubicin, taxol, ethiduim bromide, mitomycin, etoposide, tenoposide, vincristine, vinblastine, colchicine, dihydroxy anthracin dione, actinomycin D, PE40, abrin, or a glucocorticoid.

46. The antibody of claim 38, wherein the antibody has a complementarity-determining region (CDR) sequence consisting of SEQ ID NO:70 or SEQ ID NO:71.

47. A pharmaceutical composition comprising a pharmaceutical carrier and an effective amount of the antibody of claim 38.

48. A method to generate an immune response against severe acute respiratory syndrome in a mammal comprising administering to the mammal a therapeutically effective amount of the polypeptide of claim 1.

49. The method of claim 48, wherein the polypeptide consists of amino acid sequence SEQ ID NOs: 13, 14, 15, 20-59, 61-63, 66 or 69.

50. The method of claim 48, wherein the mammal is a human.

51. A method to treat severe acute respiratory syndrome in a mammal comprising administering to the mammal a therapeutically effective amount of an antibody that binds to the polypeptide of claim 1.

52. The method of claim 51, wherein the antibody specifically binds to an amino acid sequence consisting of SEQ ID NO: 61, 62, 63, 66 or 69.

53. The method of claim 51, wherein the mammal is a human.

54. A method for treating or inhibiting severe acute respiratory syndrome in a mammal comprising administering to the mammal a therapeutically effective amount of a nucleic acid encoding a S polypeptide consisting of amino acid sequence SEQ ID NO: 13, 14, 15, 20-59, 61-63, 66 or 69; or the expression cassette of claim 21.

55. The method of claim 54, wherein the mammal is a human.

56. A method to diagnose severe acute respiratory syndrome in a mammal comprising:
(a) contacting a biological sample obtained from the mammal with an antibody that binds to a polypeptide of claim 1; and
(b) determining if the antibody binds to the biological sample.

57. The method of claim 56, wherein the mammal is a human.

58. A method for making an antibody comprising: obtaining an animal that was immunized with a polypeptide having an amino acid sequence consisting of any one of SEQ ID NO: 13, 14, 15, 20-59, 61-63, 66 or 69; and isolating an antibody that binds to the polypeptide of claim 1.

59. A method to make an antibody comprising: obtaining an animal that was immunized with a coupled protein having a carrier protein coupled to (a) a polypeptide having an amino acid sequence consisting of any one of SEQ ID NOs: 13, 14, 15, 20-59, 61-63, 66 or 69, or (b) a peptidemimetic of a polypeptide having an amino acid sequence consisting of any one of SEQ ID NOs: 13, 14, 15, 20-59, 61-63, 66 or 69; and isolating an antibody that binds to a polypeptide having an amino acid sequence as set forth in SEQ ID NO: 1.

60. An aptamer that binds to an amino acid sequence as set forth in any one of SEQ ID NOs: 1, 13, 14, 15, 20-59, 61-63; or a fragment of SEQ ID NO: 1.

61. A pharmaceutical composition comprising a pharmaceutical carrier and an effective amount of the aptamer of claim 60.

62. A kit comprising packaging material and an antibody or aptamer that binds to a polypeptide having an amino acid sequence consisting of any one of SEQ ID NOs: 13, 14, 15, 20-59, 61-63, 66 or 69.

63. The kit of claim 62, further comprising a syringe.

64. A kit comprising packaging material and a therapeutically effective amount of the polypeptide of claim 1.

65. The kit of claim 64, further comprising a syringe.
